(19) United States
(12) Patent Application Publication
(10) Pub. No.: US 2019/0082678 A1
(43) Pub. Date: Mar. 21, 2019

(54) COMPOSITIONS AND METHODS FOR TISSUE PRESERVATION AT AMBIENT OR SUBNORMOTHERMIC TEMPERATURES

(71) Applicants: PRESIDENT AND FELLOWS OF HARVARD COLLEGE, Cambridge, MA (US); THE UNITED STATES GOVERNMENT AS REPRESENTED BY THE DEPARTMENT OF VETERAN AFFAIRS, Washington, DC (US)

(72) Inventor: Hemant THATTE, Medfield, MA (US)

(21) Appl. No.: 15/735,089

(22) PCT Filed: Jun. 9, 2016
(86) PCT No.: PCT/US2016/036738

§ 371 Date: Dec. 8, 2017

Related U.S. Application Data
(60) Provisional application No. 62/173,184, filed on Jun. 9, 2015.

(51) Int. Cl. A01N 1/02; A61K 33/06; A61K 38/28; A61K 31/198; A61K 31/715

(52) U.S. Cl. CPC A01N 1/0226; A01N 1/0278; A01N 1/0247; A61K 31/715; A61K 38/28; A61K 31/198; A61K 33/06

(57) ABSTRACT

Provided herein, inter alia, are methods and compositions for resuscitating, storing, and preserving the functional integrity of organs and tissues at ambient temperatures for up to 24 hours. Metabolic function is maintained by sustaining ATP levels, mitochondrial function, prevention of edema, and by regulating calcium, sodium, potassium, magnesium, and chloride ions.

### CROSS-REFERENCE TO RELATED APPLICATIONS

This application claims priority to U.S. Provisional Patent Application No. 62/173,184, filed Jun. 9, 2015, the disclosure of which is incorporated by reference herein in its entirety.

### GOVERNMENT INTEREST

The invention was made with government support under Merit Award number BX000817-01A1, awarded by the United States Department of Veterans Affairs. The government has certain rights in the present invention.

### FIELD OF INVENTION

This invention relates, inter alia, to compositions and methods to preserve biological tissues and organs over relatively long periods of time at ambient temperatures.

### BACKGROUND

The major obstacles in organ transplantation are the limited availability of donor organs and the poor quality of donor organs due to deterioration during storage. This is particularly true with donor hearts, which can currently be stored ex vivo for only 4 to 6 hours. Currently utilized solutions for extracorporeal heart storage are based on the prevention of edema and slowing down of metabolism (metabolic degeneration) during hypothermic storage at temperatures at or near 4° C. However, such low temperatures inevitably result in tissue and cellular damage which becomes irreversible after approximately 6 hours of storage.

### SUMMARY

The compositions and methods described herein represent a significant improvement and advantage over existing methods for organ storage and/or preservation of organs from Beating Heart Donors (BHD), marginal, and Donation after Cardiac Death (DCD) donors. For example, the improved storage/preservation solutions, e.g., improved Somali (iSomah), permit storage and perfusion of organs at ambient (25° C.) as well as at sub-ambient (>4 and <25° C.). Exposure of an organ, e.g., a heart, to hypothermia leads to injury and even damage to the organ. Moreover, the transition from a beating heart at normothermia (36.4-37.1° C., e.g., 36.4° C. 36.5° C., 36.6° C., 36.7° C. 36.8° C., 36.9° C., 37° C., or 37.1° C.) to a non-beating heart at hypothermic temperatures, e.g., 4° C., is also stressful for cardiac tissue. However, rather than complete stasis observed at 4° C. due to hypothermia, if a heart continues to demonstrate slow contractions or movements in a novel solution that facilitates storage at room temperature, e.g., approximately 20° C. (21° C.±+4° C.) it would deplete cellular high-energy phosphate stores (ATP+CP) and beat to exhaustion leading to associated degenerative changes such as loss of cellular homeostasis, calcium overload and induction of apoptosis and/or necrosis resulting in untransplantable organ known as a “stone heart.” Therefore, the concentration of potassium ions and magnesium ions in the solutions described herein are increased (compared to other storage or cardioplegia solutions) to induce temporary paralysis of the heart tissue during subnormothermic storage (e.g., 10-25° C.)), while simultaneously maintaining HEP synthesis, cellular homeostasis, nitric oxide generation (additionally by chelating toxic ammonium ions generated from transaminase reactions; see FIG. 33). This results in increased activity of the nitric oxide synthetic pathway in all tissues and organs and accompanying physiological control of edema.

Cardioplegia is an intentional and temporary cessation of cardiac activity. Such a temporary arresting of the heartbeat is carried out by any of various methods such as by injection or infusion of chemical substances such as a cardioplegia solution. For example, the heart is stopped in such a manner for cardiac surgery. Such surgeries include bypass surgery, heart valve replacement, aorta repair surgery, and heart transplantation etc.

In addition to improving/preserving the condition, e.g., reducing tissue damage of the heart organ, the solutions provide other advantages such as reduced cost, potentially reduced need for immunosuppression following transplantation because of minimal endothelial and tissue damage to the heart, reduced need for inotropic support (e.g., drugs are not required to increase heart contraction) and incessant electroversions to maintain sinus cardiac function, decreased CPB time, as well as stay in the ICU and hospital (hence, decreased costs). Additionally, use of the organ preservation solutions provided herein result in decreased patient morbidity and improved long-term outcomes and thus most importantly improved quality of life of the patients. Once the heart is removed from the solution, the solution is flushed out of the heart tissue during surgery. The organ then goes into sinus conversion and resumes beating upon reperfusion with blood and rewarming, in vitro; or upon release of cross-clamp upon transplantation and during rewarming the patient to normothermia.

Provided herein, inter aliu, are compositions, methods, and kits for preserving or resuscitating biological tissues or organs at ambient temperatures.

Accordingly, in some aspects, provided herein are compositions for preserving or resuscitating biological tissue or organs comprising: a physiological salt solution, glucose (or other sugars such as lactose, maltose, and/or ribose) at concentrations of any of about 5-10 mM, such as about 5 mM, 6 mM, 7 mM, 8 mM, 9 mM, or 10 mM), and one or more of glutathione, ascorbic acid, arginine, citrulline malate (or, optionally, citrulline or salts thereof and/or malic acid or salts thereof), adenosine, creatine (such as creatine orotate or creatine monohydrate or salts thereolf), orotic acid (or salts thereof) carnosine (such as, L-carnosine), carnitine (such as, L-carnitine), orotic acid, and/or dichloroacetate at concentrations of between 0 mM to about 5 mM), wherein the physiological salt solution comprises at least 20 mM potassium ions and at least 37 mM magnesium ions. The organ storage solutions described herein containing at least 20 mM potassium ions and/or at least 37 mM magnesium ions exhibit superior properties with respect to protecting and preserving organs during storage and during procedures such as cardioplegia over a more varied range of storage conditions (for example, temperature) compared to previously described organ storage solutions (see U.S. Pat. No. 8,211,628, the disclosure of which is incorporated by reference herein). As such, the storage solutions disclosed herein are referred to as “improved Somah” (iSomah).

In other aspects, provided herein are compositions for preserving mammalian organs comprising: a physiological salt solution, and one or more of a five or six carbon sugar (such as, ribose, glucose or dextrose), glutathione, ascorbic acid, arginine, citrulline (such as, citrulline malate), malic acid, adenosine, creatine (such as creatine orotate or creatine monohydrate or salts thereof), carnosine (such as, L-carnosine), carnitine (such as, L-carnitine), orotic acid, and/or dichloroacetate, wherein the composition or the organ is maintained at a temperature of 21±4° C.

In some embodiments of any of the embodiments disclosed herein, the composition or the organ is maintained at a temperature of 21±4° C. In some embodiments of any of the embodiments disclosed herein, the physiological salt solution comprises at least 20 mM potassium ions and at least 37 mM magnesium ions. In some embodiments of any of the embodiments disclosed herein, the composition further comprises insulin. In some embodiments of any of the embodiments disclosed herein, insulin is added to the composition just prior to use. In some embodiments of any of the embodiments disclosed herein, the physiological salt solution comprises one or more salts selected from the group consisting of potassium phosphate, potassium chloride, sodium chloride, sodium bicarbonate, calcium chloride, sodium phosphate, magnesium chloride, magnesium sulfate. In some embodiments of any of the embodiments disclosed herein, the composition comprises 0.44-10 mM of potassium phosphate. In some embodiments of any of the embodiments disclosed herein, the composition comprises 4-65 mM of potassium chloride. In some embodiments of any of the embodiments disclosed herein, the composition comprises 80-135 mM sodium chloride. In some embodiments of any of the embodiments disclosed herein, the composition comprises 2-25 mM sodium bicarbonate. In some embodiments of any of the embodiments disclosed herein, the composition comprises 0-1.5 mM calcium chloride. In some embodiments of any of the embodiments disclosed herein, the composition comprises 0.15-30 mM sodium phosphate. In some embodiments of any of the embodiments disclosed herein, the composition comprises 0.5-45 mM magnesium chloride. In some embodiments of any of the embodiments disclosed herein, the composition comprises 0.5-1.5 mM magnesium sulfate.

For example, in addition to a storage and/or preservation solution, the physiologically-compatible solutions (modified Somah) described herein are useful as a cardioplegia solution for arresting hearts over a temperature range encountered during open heart surgery and for donor and recipient hearts for and during transplantation. For such applications at 4-10° C., the solution contains 20 mM potassium ions, e.g., 20 mM KCl, final concentration; at 10-25° C., the solution contains 20 mM potassium ions. e.g., 20 mM KCl and 37 mM magnesium ions, e.g., 37 mM MgCl₂, final concentration; at 25-37° C. the solution contains 45 mM potassium ions. e.g., 45 mM KCl. and 37 mM Magnesium ions, e.g., 37 mM MgCl₂. In other embodiments, at 25-37° C. the solution contains 25 mM potassium ions, e.g., 25 mM KCl, and 37 mM Magnesium ions, e.g., 37 mM MgCl₂.

Exemplary temperature ranges for organ arrest (e.g., heart cardioplegia) as well as organ storage (e.g., heart, lung, or other organ storage ex vivo) as well as potassium ion and magnesium ion concentrations (e.g., concentrations of KCl and MgCl₂) are described below.

In one embodiment, hearts are arrested with Somah cardioplegia containing 20 mM KCl (range 4.0-65 mM) and 37 mM MgCl₂ (range 1.5-45 mM) at 4-37° C. and preserved in the same solution at 4-37° C. for transplant. In another embodiment, lungs are preserved in modified Somah containing 7.5 mM KCl and 2 mM MgCl₂ at 4-37° C. as well as in Somah containing 20 mM KCl (range 4.0-65 mM) and 37 mM MgCl₂ (range 1.5-45 mM) at 4-37° C. for transplant.

In other aspects, provided herein are methods for storing, preserving or resuscitating a biological tissue or organ, comprising bringing said biological tissue or organ into contact with any of the compositions disclosed herein or above. In some embodiments, the composition is maintained at a temperature of 10-21±4° C. In some embodiments of any of the embodiments disclosed herein, the biological tissue or organs are stored or preserved for 24-72 hours. In some embodiments of any of the embodiments disclosed herein, the biological tissue or organ is selected from the group consisting of heart, kidney, liver, stomach, spleen, skin, pancreas, lung, brain, eye, intestines, and bladder. In some embodiments of any of the embodiments disclosed herein, the amount of high energy phosphates are higher in the biological tissue or organ following preservation or resuscitation compared to biological tissue or organs not contacted with the composition. In some embodiments of any of the embodiments disclosed herein, the organ is a heart. In some embodiments of any of the embodiments disclosed herein, coronary blood flow is higher in the biological tissue or organ following preservation or resuscitation compared to biological tissue or organs not contacted with the composition. In some embodiments of any of the embodiments disclosed herein, one or more of percent fractional area change, ejection fraction, and/or stroke volume and cardiac output is increased in hearts following preservation or resuscitation compared to hearts not contacted with the composition.

In further aspects, provided herein are methods for producing a composition for storing, preserving or resuscitating biological tissue or organs comprising combining a physiological salt solution and one or more of glucose (11-25 mM), glutathione, ascorbic acid, arginine, citrulline (such as citrulline malate), adenosine, creatine (such as creatine orotate or creatine monohydrate or salts thereof (0.5-10 mM)), orotic acid (0.5-2.5 mM), carnosine (such as, L-carnosine), carnitine (such as, L-carnitine), and/or dichloroacetate, wherein the physiological salt solution comprises at least 20 mM potassium ions and at least 37 mM magnesium ions. In some embodiments, the method further comprises combining the composition with insulin. In some embodiments, insulin is combined just prior to use. In some embodiments of any of the embodiments disclosed herein the composition is maintained at a temperature of 10-21±4° C. In some embodiments of any of the embodiments disclosed herein, the physiological salt solution comprises one or more salts selected from the group consisting of potassium phosphate, potassium chloride, sodium chloride, sodium bicarbonate, calcium chloride, sodium phosphate, magnesium chloride, magnesium sulfate. In some embodiments of any of the embodiments disclosed herein, the physiological salt solution comprises 0.44-10 mM of potassium phosphate. In some embodiments of any of the embodiments disclosed herein, the physiological salt solution comprises 4-65 mM of potassium chloride. In some embodiments of any of the embodiments disclosed herein, the physiological salt solution comprises 80-135 mM sodium chloride. In some embodiments of any of the embodiments disclosed herein, the physiological salt solution comprises 2-25 mM sodium bicarbonate. In some embodiments of any of the embodiments disclosed herein, the physiological salt solution comprises 0-1.5 mM calcium chloride. In some embodiments of any of the embodiments disclosed herein, the physiological salt solution comprises 0.15-30 mM sodium phosphate. In some embodiments of any of the embodiments disclosed herein, the physiological salt solution comprises 0.5-45 mM magnesium chloride. In some embodiments of any of the embodiments disclosed herein, the physiological salt solution comprises 0.5-1.5 mM magnesium sulfate.

In another aspect, provided herein are kits comprising: a physiological salt solution and one or more of glucose, glutathione, ascorbic acid, arginine, citrulline (such as citrulline malate), adenosine, creatine (such as creatine orotate or creatine monohydrate or salts thereof), carnosine (such as, L-carnosine), orotic acid, carnitine (such as, L-carnitine), dichloroacetate, and/or insulin, wherein the physiological salt solution comprises at least 20 mM potassium ions and at least 37 mM magnesium ions. In some embodiments, the physiological salt solution comprises one or more salts selected from the group consisting of potassium phosphate, potassium chloride, sodium chloride, sodium bicarbonate, calcium chloride, sodium phosphate, magnesium chloride, magnesium sulfate. In some embodiments of any of the embodiments disclosed herein, the kit comprises 0.4-10 mM of potassium phosphate. In some embodiments of any of the embodiments disclosed herein, the kit comprises 4-65 mM of potassium chloride. In some embodiments of any of the embodiments disclosed herein, the kit comprises 80-135 mM sodium chloride. In some embodiments of any of the embodiments disclosed herein, the kit comprises 2-25 mM sodium bicarbonate. In some embodiments of any of the embodiments disclosed herein, the kit comprises 0-1.5 mM calcium chloride. In some embodiments of any of the embodiments disclosed herein, the kit comprises 0.15-030 mM sodium phosphate. In some embodiments of any of the embodiments disclosed herein, the kit comprises 0.5-45 mM magnesium chloride. In some embodiments of any of the embodiments disclosed herein, the kit comprises 0.5-1.5 mM magnesium sulfate.

In still other aspects, provided herein are compositions for storing, preserving, or resuscitating biological tissue or organs comprising: 7 mM potassium chloride, 0.44 mM potassium phosphate (monobasic), 0.5 magnesium chloride (hexahydrate), 0.5 mM magnesium sulfate (heptahydrate), 125 mM sodium chloride, 5 mM sodium bicarbonate, 1.3 mM calcium chloride, 0.19 mM sodium phosphate (dibasic; heptahydrate), 11 mM D-glucose, 1.5 mM glutathione (reduced), 1 mM ascorbic acid, 5 mM L-arginine, 1 mM L-citrulline malate, 2 mM adenosine, 0.5 mM creatine orotate, 2.0 mM creatine monohydrate or salts thereof, 10 mM L-carnosine, 10 mM L-carnitine, and 0.5 mM dichloroacetate. In some embodiments, the compositions further comprise 100 units/L insulin. In some embodiments, the insulin is added to the composition just prior to use. In some embodiments of any of the embodiments provided herein, the composition is maintained at a temperature of 21±4° C.

Advantages of the improved organ storage preservation solutions described herein include: (1) preservation of hearts at hypothennia (4° C.) is superior to current clinically used solutions Celsior and UWS; (2) preserves hearts in fully functional state at ambient temperatures, while the clinically used solutions (such as Celsior and UWS) cannot; (3) preserves heart in excellent condition over the temperature range of 4-25° C.; others cannot (heart metabolism and homeostasis in preserved or accentuated over this temperature range but not in other solutions; (4) hearts require minimal stimulatory interventions for reanimation due to preservation and synthesis of high energy phosphates over the temperature range of storage; hearts in other solutions cannot; (5) facilitates functional preservation of (Beating Heart Donor) BHD and (Donation after Cardiac Death) DCD hearts for 24 hours and other organs for over 72 hours at over the temperature range; while other solutions cannot.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts the Somah Device, a custom-built apparatus specifically designed for extracorporeal reanimation of hearts was used. Two circuits were prepared, first for the antegrade perfusion of coronaries through aorta during initial reperfusion of the hearts, and second for the perfusion of hearts through PVs in a working heart. Briefly, in Circuit 1 (green), the perfusate was pumped from heart chamber to the oxygenator-heat-exchanger system, and eventually into the aorta for perfusion of coronaries. The return of perfusate to the heart chamber through PA completed this circuit. In Circuit 2 (red), blood pumped from the heart chamber to the oxygenator—heat exchanger system was collected in a pre-load bag from where it drained into the PVs by gravity. The pressures/flows were adjusted by altering the height of the pre-load bag. This circuit was diverted into two components. The first component was the part of perfusate that entered the coronaries and returned to the heart chamber through the PA. The second component was formed by the perfusate that continued through the aorta into the after-load chamber, from where the perfusate was allowed to return to the heart chamber by gravity. A CDI monitor was incorporated into the system in addition to the oxygenator-heat-exchanger system for real-time monitoring of changes in perfusate pH, temperature, PO₂, PCO₂, K⁺ and HCO₃⁻. Pressures and flows were recorded at various points in the two circuits. The pressure and flow data were acquired and monitored in real time using a computer and HMI software specifically written for the Somah device (Comdel, Inc., Wahpeton, N. Dak.). DAS, data acquisition system. Video of the Somah Device and working heart can be seen at https://www.youtube.com/watch?v=PTga7aeuVzk.

FIG. 2 depicts a flow diagram showing the experimental design. The illustration shows the general experimental design of this study, starting from intra-operative cardioplegia for cardiac arrest to the end of the ex vivo heart reperfusion experiment.

FIG. 3 depicts high-energy phosphates during storage. The graph shows the alterations in HEP levels during 5-hour storage in Somah, Celsior and UWS group hearts. Asterisk: significantly higher than controls; dagger: significantly lower than controls.

FIG. 4A, and FIG. 4B depict cardiac enzymes upon reperfusion. Graphs showing the release of the cardiac enzymes creatine kinase and troponin I into the ex vivo circulation upon reperfusion of hearts with the Somah device are depicted in FIG. 4A and FIG. 4B, respectively. Asterisk: significantly higher versus the other groups.

FIG. 5A and FIG. 5B depict the metabolic shift upon reperfusion. Graphs showing alterations in myocardial oxygen consumption and lactate ratio in hearts within 30 minutes of reperfusion in the Somah, Celsior and UWS groups are depicted in FIG. 5A and FIG. 5B, respectively. Asterisk: significantly higher than baseline.

FIG. 6A, FIG. 6B, FIG. 6C, and FIG. 6D depict two-dimensional echocardiographic analysis during extracorporeal perfusion showing functional parameters. Percent fractional area change is depicted in FIG. 6A; ejection fraction is depicted in FIG. 6 B; and stroke volume is depicted in FIG. 6C. The findings are deduced from 2D echocardiography on the Somah, Celsior and UWS group hearts. FIG. 6 D depicts alteration in left ventricular anterior wall and septal wall thicknesses upon reperfusion of hearts in the Somah. Celsior and UWS groups. Asterisk: significantly lower than in the Somah group.

FIG. 7 depicts a flow diagram of the experimental design. Illustration shows the general experimental design of this study, starting from intraoperative cardioplegia for cardiac arrest to the end of ex vivo heart reperfusion experiment.

FIG. 8 depicts an assessment of edema during 5-h heart storage. Heart biopsies were obtained for evaluation of edema and ischemic changes by electron microscopy (EM) (upper panels; magnification—8000×; inset in the first EM image shows a cardiomyocyte nuclei representative of reversible change seen in all the three groups, demonstrating partial condensation of chromatin material below the nuclear membrane) and histopathology (middle panels; magnification-400×) in the 4° C. (left), 13° C. (center) and 21° C. (right) group hearts; representative images. The lower graphs show the alteration in heart weights after storage, from prior at harvest weights in the three groups. M, mitochondria; SR, sarcoplasmic reticulum; G, glycogen granules.

FIG. 9A and FIG. 9B depict cardiac metabolism in working hearts. Myocardial O₂ consumption (MVO₂) is depicted in FIG. 9A and lactate ratio is depicted in FIG. 9B upon perfusion of hearts stored at 4° C. 13° C. and 21° C. MVO₂ and lactate ratios were determined from the differences in the respective parameters in the outflow and inflow perfusate samples. Baseline=60 min after reperfusion, at hemodynamic steady state; 30 min=at peak performance, 90 min after reperfusion. Each bar represents mean±SEM of n=6 for each Somah and n=5 for Celsior group, respectively. ‡Significant change, Celsior versus Somah groups at corresponding time points; * Significant change from baseline (p<0.05).

FIG. 10A and FIG. 10B depict release of creatine kinase (CK) and cardiac troponin-I (cTnI) upon reperfusion. CK (FIG. 10A) and cTnI (FIG. 10B) levels were determined in perfusate, 5 min and 90 min (peak performance) after start of reperfusion of hearts stored at 4° C., 13° C., and 21° C.; n=6 for each Somah group, and n=5 Celsior group. Significant change from Celsior (p<0.05).

FIG. 11 depicts a two-dimensional echocardiography (2D Echo) image procured during in vitro experiments using trans-esophageal echocardiography (TEE) probe. 2D Echo images during ex vivo experiments were acquired using TEE probe. Images show end-diastolic (upper panels) and end-systolic (lower panels) images (short-axis views) acquired with TEE probe at the papillary muscle level of the left ventricle, at peak performance upon in vitro coronary reperfusion of hearts stored either at 4° C. (left column), 13° C. (central column) or 21° C. (right column). Representative images of independent experiments (n=6 for each group).

FIG. 12A. FIG. 12B, and FIG. 12C depict viability evaluation of stored hearts. Cardiac biopsies were taken either immediately on procurement (as depicted in FIG. 12A; controls) or before (as depicted in FIG. 12B) or after (as depicted in FIG. 12C) reperfusion of hearts donated after cardiocirculatory death preserved in Somah for 24 hours at 4° C., 10° C., 21° C., or 37° C. Green fluorescence (lower panels) indicates cell viability; red fluorescence (upper panels), compromised cardiomyocytes. In hearts preserved in Somah for 24 hours (as depicted in FIG. 12B), a robust green fluorescence of live cells was apparent in all temperature groups. The red fluorescence was noted at 4° C., 10° C., and 37° C. Representative images of independent experiments; magnification 320×.

FIG. 13A. FIG. 13B, FIG. 13C, FIG. 13D, and FIG. 13E depict mitochondrial membrane polarization in stored hearts. Mitochondrial membrane polarization in controls is depicted in FIG. 13A; hearts donated after cardiocirculatory death preserved in Somah for 24 hours at different temperatures is depicted in FIG. 13B; or after reperfusion is depicted in FIG. 13C. The ratio of polarized to depolarized mitochondria (n=3 in each group) after 24-hour storage (as depicted in FIG. 13D) and upon reperfusion (as depicted in FIG. 13E) was unaltered between each temperature group as well as upon reperfusion. Mitochondrial polarization was at equilibrium in all temperature groups. Representative images, 320× magnification.

FIG. 14A and FIG. 14B depict high-energy phosphate syntheses in stored hearts. Graphs show adenosine triphosphate (ATP; depicted in FIG. 14A) and Creatine phosphate (CP; depicted in FIG. 14B) concentration in hearts donated after cardiocirculatory death at procurement (control), after preservation in Somah for 24 hours at different temperatures and upon simulated reperfusion. In all temperature groups except 37° C., both ATP and CP synthesis was significantly increased (P<0.005) after 24-hour storage in Somah. Upon reperfusion, hearts stored at 4° C. and 10° C. demonstrated a decrease in ATP synthesis (P<0.005) but CP synthesis was unaltered. In hearts preserved at 21° C. and 37° C., reperfusion resulted in an increase in ATP synthesis (P<0.001) highly significant at 21° C. while CP synthesis was significantly increased only in 21° C. group (P<0.005). Error bars represent standard error of means. *Significant change from control. ‡Significant change from preperfusion (24-hour storage) levels.

FIG. 15 depicts Structural and contractile components of cardiomyocytes. Hearts donated after cardiocirculating death were preserved in Somah for 24 hours at 4° C., 10° C., 21° C., or 37° C. prior to reperfusion. Left ventricular biopsies were taken before (pre) and after (post) simulated reperfusion. Resolution of myosin heavy (H) and light (L), actinin, actin, and troponin C was investigated. Control biopsies were taken immediately after procurement of the hearts. Structural and contractile proteins were well preserved at 21° C. but were differentially lost in other temperature groups. Upon reperfusion in the 21° C. group, the myosin light chain protein migrated to a higher level than normal, possibly indicative of phosphorylation.

FIG. 16 depicts a flow diagram of experimental design. Illustration shows the general experimental design of this study, starting from intraoperative cardioplegia for cardiac arrest to the end of ex vivo heart reperfusion experiment.

FIG. 17A, FIG. 17B, and FIG. 17C are graphs depicting high energy phosphate levels in hearts arrested and stored in SOMAH. Cardiac tissue biopsies from left ventricle were obtained for determination of HEP including ATP and CP levels in 4 and 21° C. SOMAH cardioplegia group hearts pre and post 5-hour storage. There was a temperature of cardioplegia arrest dependent increase in HEP concentrations in the hearts. FIG. 17A depicts Control; FIG. 17AB depicts 5 hour storage; and FIG. 17C depicts normalized values (5 hours with respect to 0 hour). Each bar represents mean±SEM of n=5 for each group. * Significantly different from 4° C. cardioplegia group hearts.

FIG. 18A, FIG. 18B, and FIG. 18C are graphs depicting release of creatine kinase and cardiac troponin-I upon reperfusion. CK (depicted in FIG. 18A). AST (depicted in FIG. 18B) and cTnI (depicted in FIG. 8C) levels were determined in perfusate, 5 min and 90 min (peak performance) after start of reperfusion of 4 and 21° C. cardioplegia hearts; n=5 for each SOMAH group. *Significant change from 5 minutes (p<0.05); * Significantly different from 4° C. cardioplegia group hearts at similar time point.

FIG. 19A and FIG. 19B are bar graphs depicting cardiac metabolism in working hearts. FIG. 19A depicts myocardial O₂ consumption while FIG. 19B depicts lactate ratio (B) upon perfusion of 4 and 21° C. cardioplegia hearts. MVO₂ and lactate ratios were determined from the differences in the respective parameters in the outflow and inflow perfusate samples. Baseline=60 min after reperfusion, at hemodynamic steady state; 90 min=at peak performance. Each bar represents mean+SEM of n=5 for each group.

FIG. 20 depicts gross appearance of livers stored in UWS or Somah solutions. Morphology of DCD livers. Liver stored in UWS showed significant discoloration within 1 hour of storage. In contrast, livers stored in Somah maintained their color and morphology throughout the 72-hour storage period. All livers were progressively biopsied for further analysis. Representative images: UWS n=7; Somah n=6

FIG. 21 depicts histopathology of livers at 6, 24 and 72 hrs stored in University of Wisconsin (UWS) and Somah solutions. Note that bile ductules show mucosal ulceration and disorganized, heaped, condensed nuclei in livers stored in UWS. These changes were seen as early as 6 hrs. In contrast, livers stored in Somah for 72 hrs showed normal appearing bile ductules in the portal region with clear, rounded uniform lumen and intact mucosa with regular basal nuclei. Also note that in the upper panels, some periportal hepatocytes show ballooning degeneration and apoptotic nuclei (arrows), while periportal hepatocytes showed normal cellular boundaries and heterochromatic, open-faced nuclei with nucleoli in livers stored in Somah (arrows). Asterisks indicate bile ducts and ductules of different calibers (all images, ×200).

FIG. 22 depicts a high power view (×400) of bile ductules obtained from livers cold-stored in UWS and Somah at 6 hrs. Note the regularly arranged basal nuclei and clear lumen in a medium sized bile ductule seen at 0 hr (arrow, left panel). In contrast, note the polychromatic appearance of the ductular nuclei, including some condensed nuclei and reactive (proliferative) nuclear changes in the 3 o'clock position of liver stored in UWS. Note the sloughed material obstructing the ductular lumen and non-uniformly stained and ragged appearance of the mucosa. In contrast, lumina of bile ductules were regular appearing with intact mucosa in Somah stored livers. These changes were uniformly seen in bile ducts of different diameters (asterisks). Green asterisks indicate portal veins/venules (×400).

FIG. 23A and FIG. 23B are bar graphs depicting changes in pH, lactate and glucose levels in livers during storage. Graphs show time-dependent alterations in pH (upper panel), lactate (middle panel) and glucose (lower panel) levels in UWS (FIG. 23A) and Somah (FIG. 23B) solutions during extracorporeal storage of DCD livers. Metabolic parameters were temporally assessed in the storage solution during extracorporeal storage of DCD livers in UWS and Somah for 72 hours at 4° C.

FIG. 24A and FIG. 24B depict oxygen consumption and co₂ production in Stored Livers. FIG. 24A shows the extent of oxygen consumption and FIG. 24B shows CO₂ production during extracorporeal storage of livers in UWS and Somah solutions at 0, 6, 24 and 72 hour time points. *Significant change from baseline levels in Somah.

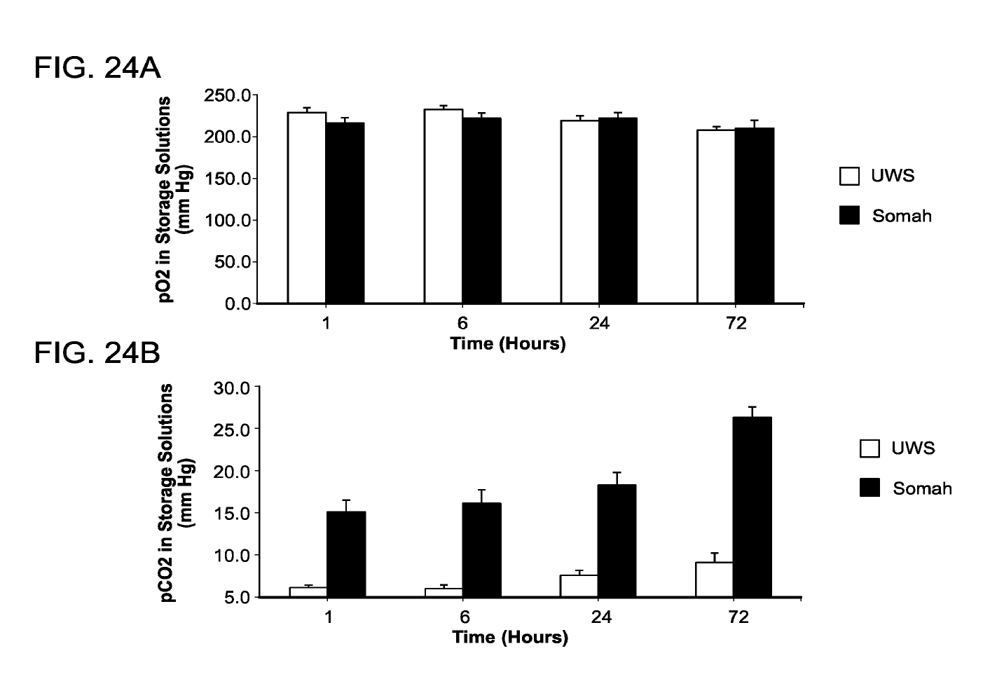

FIG. 25 depicts graphs showing total phosphates in stored livers. Graphs show time-dependent changes in ATP, CP and total phosphate levels in liver tissue during long-term extracorporeal storage of DCD livers in UWS (upper) and Somah (lower). *p<0.05, Compared with 1 Hour.

FIG. 26 depicts graphs showing release of liver enzymes during organ storage. Release of liver enzymes during extra-corporeal preservation of DCD livers was determined in the respective UWS or Somah solutions at time 0, 6, 24 and 72 hours. ALT (upper), AST (middle) and CK (lower) levels were evaluated.

FIG. 27 depicts bar graphs showing reperfusion induced release of liver enzymes. Release of liver enzymes during extra-corporeal reperfusion of DCD Somah livers was determined in the perfusate (HV) at time 0 (single pass), 0.5 and 2 hours. ALP, GGT, AST, ALT and CK levels were evaluated. Due to intra variability of enzymes in 72-hour stored blood in the reconstituted perfusate, data was normalized to time 0 hour values; mean±SEM, from independent experiments.

FIG. 28 depicts a bar graph showing reperfusion induced synthesis and release of albumin by livers stored in Somah for 72-hours. Somah livers temporally synthesized and released albumin in the perfusate (HV). Increase in albumin synthesis was highly significant at 0.5 hour (P<0.03) and at 2 hours (P<0.01). Values represent mean+SEM from independent experiments.

FIG. 29A and FIG. 29B depict gross morphology of kidneys stored in UW (FIG. 29A) or Somah (FIG. 29B). Kidneys were stored for 72 hours and images were obtained for gross morphological evaluation and biopsies taken for histopathology at time 0, and 6, 24 and 72 hours of extracorporeal preservation at 4° C. Kidneys flushed with UW displayed a mottled appearance with patchy discoloration at all time points (a). Kidneys flushed with Somah displayed uniform color and smooth morphology without patchy changes (d). Histological examination showed absence of interstitial edema in both UW (b, 200×; c, 400×) and Somah (e, 200×; f, 400×) stored DCD kidneys at all the investigated time points. Higher magnifications showed a greater propensity for nuclear hyperchromacity of tubular epithelial cells of UW kidneys (c) as compared to Somah kidneys (f) at 6, 24 and 72 hour time points.

FIG. 30A, FIG. 30B, FIG. 30C, FIG. 30D, and FIG. 30E depict bar graphs showing alterations in metabolic parameters in the UW or Somah solutions storing DCD kidneys over a 72-hour period. FIG. 30A shows pH; FIG. 30B shows glucose; FIG. 30C shows lactate; FIG. 30D shows pO₂ and FIG. 30E shows pCO₂.

FIG. 31 depicts line graphs showing alterations in energy metabolism in the UW (left) and Somah (right) stored DCD kidneys during the 72 hour extracorporeal preservation period. *Significantly different from Time 0 (p<0.05).

FIG. 32 depicts bar graphs showing time-dependent alterations in expression of caveolin, endothelial nitric oxide synthase (eNOS), von-Willebrands factor (vWF) and erythropoietin (EPO) proteins in DCD kidneys stored in either UW or Somah solution for 72 hours.

FIG. 33 is a chart depicting ammonia production and utilization in cells exposed to Somah.

### DETAILED DESCRIPTION

What is urgently needed in the art is an organ preservation storage solution that facilitates preservation of an organ from various groups of donors over a broad subnormothermic temperature range (4-25° C.), thus preventing tissue injury due to storage at extreme hypothermia (4° C.) prior to transplant. The components of the solution should preserve cardiac (and other organ) structure and function by providing ionic balance, energy substrates, chelation of ammonia into substrates for nitric oxide synthase, metabolic modulation for generation of high-energy phosphates (HEP), free radical scavenging, anti-oxidants, reducing agents, intra and extracellular H⁺ chelation, and attenuation of edema by modulation of hemichannels and aquaporins during storage. The storage solution should also facilitate attenuation of ischemia-reperfusion injury (IRI) by preloading with selective, synergistic constituents during hypoxic storage to counterbalance the detrimental effects of the initially hyperoxic state post-reperfusion, and consequently prevent reperfusion injury and perpetuate uneventful rapid transition to normoxic state, aerobic metabolism and optimum mechanical function. An ideal solution would synergistically 1) preserve the organ during ischemic storage; 2) prime the organ with metabolites for rapid conversion from hyperoxic to normoxic state, for sustained electromechanical work upon reperfusion; and 3) prevent ischemia-reperfusion (IR) injury. Such a solution would have the potential to greatly extend temporal storage for extracorporeal preservation of donor organs prior to transplantation into recipients.

The invention described herein provides, inter alia, compositions for preserving mammalian organs and tissues as well as methods and kits for utilizing the same. While any mammalian organ or tissue can be preserved in the presently described compositions using the instantly described methods, the benefits of storing extracorporeal hearts are particularly advantageous. In contrast to currently available compositions and techniques for preserving extracorporeal hearts, prior to transplantation into recipients, the compositions and methods of the present invention permit ex vivo storage for 24-72 hours following removal from a donor. In further contrast to currently available compositions for heart preservation, which require storage at near freezing temperatures, the compositions and methods of the instant invention can be stored at ambient temperatures without accumulation of significant amounts of edema and without the characteristic cold-mediated tissue and cellular damage brought about by cold storage of hearts. The combination of increased storage time and the ability to maintain hearts at ambient temperatures during storage would allow donor hearts to be transported over longer distances over significantly increased periods of time and without the need for cold storage using the presently described compositions. Due to the fact that donor hearts are in short supply, the compositions and methods of the present invention have the potential to permit hearts to reach suitable transplant recipients located at more remote distances than what is currently possible.

### I. Definitions

As used herein, the term “physiological salt” refers to any salt which, when in aqueous solution at a given concentration, assists with or is required for a cellular or physiologic function. Examples of physiological salts include, without limitation, alkaline and alkaline earth metal chlorides, phosphates and sulfates, such as, KCl, NaCl, MgCl₂, MgSO₄, and mixtures thereof.

A “subject” can be a vertebrate, a mammal, or a human. Mammals include, but are not limited to, farm animals, sport animals, pets, primates, mice and rats. In one aspect, a subject is a human.

As used herein, “normothermic temperature” refers to any temperature in the range of about 36.4-37.1° C., e.g., 36.4° C., 36.5° C., 36.6° C., 36.7° C., 36.8° C., 36.9° C., 37° C., or 37.1° C. “Ambient temperature” or “subnormnothermic temperature,” as used herein, refers to temperatures in the range of 10-21±4° C., or in other embodiments, temperatures in the range of 21±2° C., such as any of about 6° C., 7° C., 8° C., 9° C., 10° C., 11° C., 12° C., 13° C., 14° C., 15° C., 16° C. 17° C., 18° C. 19° C., 20° C., 21° C., 22° C. 23° C., 24° C., or 25° C. “Hypothermic temperatures” or “hypothermia” refers to temperatures in the range of about 0° C. to about 5° C., such as any of about 0° C., 1° C., 2° C., 3° C., 4° C., or 5° C.

Unless defined otherwise herein, all technical and scientific terms used herein have the same meaning as commonly understood by one of ordinary skill in the art to which this invention pertains.

As used herein, the singular terms “a.” “an,” and “the” include the plural reference unless the context clearly indicates otherwise.

The transitional term “comprising,” which is synonymous with “including,” “containing,” or “characterized by,” is inclusive or open-ended and does not exclude additional, unrecited elements or method steps. By contrast, the transitional phrase “consisting of excludes any element, step, or ingredient not specified in the claim. The transitional phrase” consisting essentially of limits the scope of a claim to the specified materials or steps “and those that do not materially affect the basic and novel characteristic(s)” of the claimed invention

### II. Compositions of the Invention

Currently available techniques and compositions for storage of organs, such as donor hearts, permit only around 4-6 hours of storage prior to the onset of irreversible cold-mediated tissue and cellular damage. The compositions of the present invention are solutions for preserving or resuscitating biological tissue or organs at temperatures of about 10-21±4° C., such as any of about 4° C. 5° C., 6° C., 7° C., 8° C., 9° C., 10° C., 11° C. 12° C. 13° C. 14° C., 15° C., 16° C. 17° C. 18° C., 19° C., 20° C., 21° C., 22° C., 23° C., 24° C. or 25° C., including all temperatures and ranges (such as between about 10-25° C.) falling within these values. Tissues or organs can be stored in the compositions described herein for about 24-72 hours, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 or more hours, without significant decreases in the amount of cellular high energy phosphates or without significant increases in edema. With respect to hearts stored in the compositions of the present invention prior to transplantation into a subject in need thereof, physiological measurements such as coronary blood flow, percent fractional area change, ejection fraction, and/or stroke volume are increased upon reanimation of the heart relative to hearts stored in currently utilized organ preservation solutions.

Any organ or biological tissue can be stored in the compositions described herein, for example any of heart, kidney, liver, stomach, spleen, skin, pancreas, lung, brain, eye, intestines, or bladder. In some embodiments, the stored organ is a heart.

A. Physiological Salt Solutions

The compositions of the present invention can be aqueous (i.e. water-based or solid powder based (reconstituted with distilled water prior to use) or combinations thereof solutions that include one or more of a physiological salt solution, glucose, glutathione, ascorbic acid, arginine, citrulline malate, adenosine, creatine orotate, creatine monohydrate or salts thereof, orotic acid, malic acid, carnosine, camitine, and/or dichloroacetate, wherein the physiological salt solution comprises at least 20 mM potassium ions and at least 37 mM magnesium ions. The physiological salt solution can include any salt which, when in aqueous solution at a given concentration, assists or is required for a physiologic function such as maintaining ionic concentrations inside and outside of the biological tissue or organ as well as controlling the amount of water that can traverse cellular membranes. The components of the physiological salt solution can also help to buffer and maintain a proper pH. Particular salts capable of use in the present in invention include, without limitation, potassium chloride, potassium phosphate, magnesium chloride, magnesium sulfate, calcium chloride, sodium chloride, sodium bicarbonate and sodium phosphate.

The physiological salt solution of any of the compositions disclosed herein can contain a sodium ion source. Sodium ions can be added to the physiological salt solution in the form of a sodium salt, such as, for example, one or more sodium salts selected from the group consisting of NaAlO₂, NaBO₂, NaCl, NaClO, NaClO₂, NaClO₃, NaClO₄, NaF, Na₂FeO₄, NaHCO₃, NaH₂PO₄, NaHSO₃, NaHSO₄, NaI, NaMnO₄, NaNH₂, NaNO₂, NaNO₃, NaOH, NaPO₂H₂, NaSH, Na₂MnO₄, Na₃MnO₄, Na₂N2O₂, Na₂O₂, Na₂SO₃, Na₂SO₄, Na₂S2O₄. Na₂SeO₃, Na₂SeO₄, Na₂SiO₃, Na₂Si₂O₅, Na₄SiO₄, Na₂Ti3O₇, Na₂Zn(OH)₄. NaH₂C₆H₅O₇, and Na₃PO₄. In some embodiments, sodium ions in the biological tissue and organ storage composition can be at a concentration of between about 80-145 mM, such as about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 133 mM, about 134 mM, about 135 mM, about 136 mM, about 137 mM, about 138 mM, about 139 mM, about 140 mM, about 141 mM, about 142 mM, about 143 mM, about 144 mM, or about 145 mM including all ranges and numbers falling within these values.

In other embodiments of the compositions disclosed herein, the physiological salt solution contains sodium chloride. The concentration of sodium chloride in the biological tissue and organ storage composition can be between about 80-135 mM, such as about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 1303 mM, about 134 mM, or about 135 mM, including all ranges and numbers falling within these values. In another embodiment, the biological tissue and organ storage composition can contain about 7.3 g/L sodium chloride.

In further embodiments of the compositions disclosed herein, the physiological salt solution contains sodium phosphate. The concentration of sodium phosphate in the biological tissue and organ storage composition can be between about 0.15-30 mM, such as about 0.15 mM, about 0.16 mM, about 0.17 mM, about 0.18 mM, about 0.19 mM, about 0.2 mM, about 0.21 mM, about 0.22 mM, about 0.23 mM, about 0.24 mM, about 0.25 mM, about 0.5 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM including all ranges and numbers falling within these values. In another embodiment, the biological tissue and organ storage composition can contain about 0.05 g/L sodium phosphate. Any form of sodium phosphate can be used in the present invention, including, without limitation, the dibasic heptahydrate form.

In another embodiment of the compositions disclosed herein, the physiological salt solution contains sodium bicarbonate. The concentration of sodium bicarbonate in the biological tissue and organ storage composition can be between about 2-25 mM, such as about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, including all ranges and numbers falling within these values. In another embodiment, the biological tissue and organ storage composition can contain about 0.35 g/L sodium bicarbonate.

In another embodiment of the compositions disclosed herein, the physiological salt solution contains calcium ions (for example, calcium ions supplied by calcium salts such as calcium chloride). In some embodiments, calcium ions in the biological tissue and organ storage composition can be at a concentration of between about 0-1.5 mM, such as about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, or about 1.5 mM, including all ranges and numbers falling within these values. In another embodiment, the physiological salt solution of the compositions disclosed herein contain calcium ions supplied from one or more calcium salts such as, for example, those selected from the group consisting of calcium acetate, calcium aluminates, calcium aluminoferrite, calcium aluminosilicate, calcium ammonium nitrate, calcium arsenate, calcium ascorbate, calcium azide, calcium benzoate, calcium beta-hydroxy-beta-methylbutyrate, calcium bicarbonate, calcium bisulfite, calcium borate, calcium bromate, calcium bromide, calcium carbide, calcium carbonate, calcium chlorate, calcium chromate, calcium citrate, calcium citrate malate, calcium copper titanate, calcium cyanamide, calcium diglutamate, calcium erythorbate, calcium fluoride, calcium formate, calcium funmarate, calcium glubionate, calcium glucoheptonate, calcium gluconate, calcium glycerylphosphate, calcium guanylate, calcium hexaboride, calcium hydride, calcium hydroxide, calcium hypochlorite, calcium inosinate, calcium iodate, calcium iodide, calcium lactate, calcium lactate gluconate, calcium magnesium acetate, calcium nmalate, calcium monohydride, calcium monophosphide, calcium morphenate, calcium nitrate, calcium nitride, calcium nitrite, calcium oxalate, calcium oxide, calcium pangamate, calcium perchlorate, calcium permanganate, calcium peroxide, calcium phosphate, calcium phosphide, calcium propanoate, calcium pyrophosphate, calcium silicate, calcium silicate hydrate, calcium silicide, calcium sorbate, calcium stearate, calcium sulfate, calcium sulfate, calcium sulfide, calcium sulfite, calcium tartrate, calcium titanate, calcium chloride, and calcium cyanide.

The physiological salt solution of any of the compositions disclosed herein can contain a potassium ion source. Potassium ions can be added to the physiological salt solution in the form of a potassium salt, such as, for example, one or more potassium salts selected from the group consisting of KAsO₂, KBr, KBrO₃, KCN, KCNO, KCl, KClO₃, KClO₄, KF, KH, KHCO₂, KHCO₃, KHF₂, KHS, KHSO₃, KHSO₄, KH₂AsO₄, KH₂PO₃, KH₂PO₄, KI, KlO₃, KlO₄, KMnO₄, KN₃, KNH₂, KNO₂, KNO₃, KOCN, KOH, KO₂, KPF₆, KCH₃COO, K₂Al₂O₄, K₂CO₃, K₂CrO₄, K₂Cr₂O₇, K₂FeO₄, K₂HPO₄, K₂MnO₄, KO₂, K₂O₂, K₂S, K₂SeO₄, K₂SO₃, K₂SO₄, KHSO₅, K₂S₂O₅, K₂S₂O₇, K₂S₂O₈, K₂SiO₃, K₃[Fe(C₂O₄)₃], K₄[Fe(CN)₆], K₃PO₄, and K₄Mo₂Cl₈. In some embodiments, potassium ions in the biological tissue and organ storage composition can be at a concentration of between about 4-65 mM, such as about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about or 65 mM, including all ranges and numbers falling within these values. In other embodiments of the compositions disclosed herein, the physiological salt solution contains potassium phosphate. The concentration of potassium phosphate in the biological tissue and organ storage composition can be between about 0.4-10 mM, such as about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, or about 10 mM, including all ranges and numbers falling within these values. In another embodiment, the biological tissue and organ storage composition can contain about 0.06 g/L potassium phosphate. Any form of potassium phosphate can be used in the present invention, including, without limitation, the monobasic form.

In some embodiments of the compositions disclosed herein, the physiological salt solution contains potassium chloride. The concentration of potassium chloride in the biological tissue and organ storage composition can be between about 4-65 mM, such as about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about or 65 mM including all ranges and numbers falling within these values. In another embodiment, the biological tissue and organ storage composition can contain about 0.522 g/L potassium chloride.

The physiological salt solution of any of the compositions disclosed herein can contain a magnesium ion source. Magnesium ions can be added to the physiological salt solution in the form of a magnesium salt, such as one or more magnesium salts selected from the group consisting of MgB₂, MgBr₂, MgCO₃, MgC₂O₄, MgC₆H₆O₇, MgC₁₄H₁₀O₄, MgCl₂, Mg(ClO₄)₂, MgF₂, MgH₂, Mg(HCO₃)₂, MgI₂, Mg(NO₃)₂, MgO, MgO₂, Mg(OH)₂, MgS, MgSO₃, MgSO₄, Mg₂Al₃, Mg₂Si, Mg₂SiO₄, Mg₂Si₃O₈, Mg₃N₂, Mg₃(PO₄)₂, and Mg₂(CrO₄)₂. In some embodiments, magnesium ions in the biological tissue and organ storage composition can be at a concentration of between about 0.5-45 mM, such as about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, or about 45 mM including all ranges and numbers falling within these values.

In still further embodiments of the compositions disclosed herein, the physiological salt solution contains magnesium chloride. The concentration of magnesium chloride in the biological tissue and organ storage composition can be between about 0.5-45 mM, such as about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, or about 45 mM including all ranges and numbers falling within these values. In another embodiment, the biological tissue and organ storage composition can contain about 101.00 g/L magnesium chloride. Any form of magnesium chloride can be used in the present invention, including, without limitation, the hexahydrate form.

In other embodiments of the compositions disclosed herein, the physiological salt solution contains magnesium sulfate. The concentration of magnesium sulfate in the biological tissue and organ storage composition can be between about 0.5-1.5 mM, such as about 0.5 mM, about 0.6 mM, about 0.7 mM, about 0.8 mM, about 0.9 mM, about 1 mM, about 1.1, about 1.2 mM, about 1.3 mM, about 1.4 mM, or about 1.5 mM including all ranges and numbers falling within these values. In another embodiment, the biological tissue and organ storage composition can contain about 0.123 g/L magnesium sulfate. Any form of magnesium sulfate can be used in the present invention, including, without limitation, the heptahydrate form.

B. Other Components

In addition to the physiological salt solution, compositions of the present invention can also include one or more of glucose, glutathione, ascorbic acid, arginine, citrulline (such as citrulline malate and salts thereof), adenosine, creatine (such as creatine orotate or creatine monohydrate or salts thereof), orotic acid, malic acid and salts thereof, carnosine, carnitine, dichloroacetate, and/or insulin. The solution is manufactured and sold without insulin. Insulin is added at the specified concentration at the point of use or shortly before use or prior to use. e.g., just prior to (such as any of about 30 seconds, or 1, 2, 3, 4, 5, 6, 7, 8, 9, 10, 11, 12, 13, 14, 15, 20, 25, 30, 35, 40, 45, 50, 55, 60, or more minutes prior to) infusion into an organ of a living patient or just before infusion into an ex vivo organ or submersion of the organ into the solution.

A sugar, for example, a six carbon sugar like glucose (such as D-glucose or dextrose) and/or a five carbon sugar like ribose, can serve as a substrate for the production of high energy phosphates (such as ATP) and can be included in the biological tissue and organ storage composition described herein at concentrations between about 5-25 mM, such as any of about 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM including all ranges and numbers falling within these values. In one embodiment, the concentration of glucose is about 1.98 g/L. In another embodiment, glucose is present at a concentration of about 11 mM.

Reactive oxygen species can be generated during biological tissue and organ storage; however, ascorbic acid and reduced glutathione (i.e. reducing agents) present in the solution can consume oxygen free radicals during storage. As such, both ascorbic acid and reduced glutathione can be present in the biological tissue and organ storage composition described herein at concentrations between about 0.5 mM to 3 mM, such as any of about 0.5 mM, 1 mM, 1.5 mM, 2 mM, 2.5 mM, or 3 mM, including all ranges and numbers falling within these values. In one embodiment, the concentration of ascorbic acid is about 0.178 g/L. In another embodiment, ascorbic acid is present at a concentration of about 1 mM. In some embodiments, the concentration of reduced glutathione is about 0.462 g/L. In another embodiment, reduced glutathione is present at a concentration of about 1.5 mM.

Other components of the biological tissue and organ storage compositions disclosed herein assist in the production of ATP via the tricarboxylic acid (TCA) cycle. In the citrulline malate-arginine cycle, malate (cleaved from citrulline) enters the TCA cycle to generate more ATP. Also, citrulline malate is converted to arginine and fumarate; fumarate enters the TCA cycle to facilitate more ATP production. Both malate and fumarate in TCA cycle leads to more ATP production.

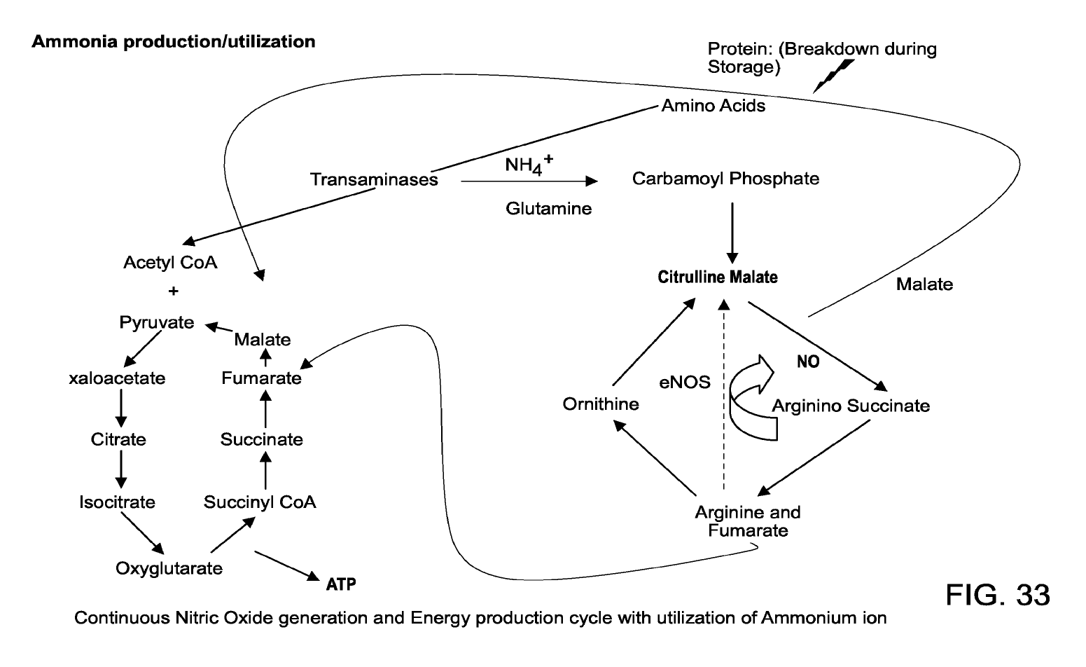

Additionally, while it is generally known that only the liver detoxifies ammonia in the urea cycle for elimination by the kidneys under normal physiological circumstances, the organ storage compositions disclosed herein may be able to drive the otherwise toxic ammonium ion into the nitric oxide synthesis pathway in most organs and tissues by inclusion of citrulline (see FIG. 33). Increased production of NO is extremely beneficial for long term storage of organs. Specifically, as soon as circulation to an organ is interrupted during harvesting and storage, transaminase (and/or protease) reactions accelerate as part of degenerative breakdown of proteins. These enzymes metabolize amino acids, thereby releasing ammonium ions that can build up in the storage solution, potentially causing toxicity and injury to the tissue. In order to negate this detrimental changecitrulline, citrulline malate, and/or salts thereof can provide a counterbalance to this increased ammonium production when included in the organ storage solutions provided herein. Without being bound to theory, is thought that ammonium ions will combine with glutamine present in cells to form carbamoyl phosphate, which is driven into the NO cycle by the formation of L-citrulline (see FIG. 33). To keep this cycle going and to prevent citrulline exhaustion citrulline malate can be included in the solution. Citrulline is metabolized to arginine (leading to NO production) and Krebs's cycle intermediates during NO production (see FIG. 33). These intermediates, such as succinate, fumarate, and malate enter into Krebs's cycle resulting in generation of additional ATP, thus further contributing to preservation of the energy state in a stored organ. Additionally, the combination of carnosine and carnitine synergistically produces a higher amount of high energy phosphates in organs stored in any of the solutions disclosed herein compared to the amount of HEPs produced using a storage solution lacking these ingredients. In another embodiment, the combination of carnosine, carnitine, glucose, and creatine synergistically produces a higher amount of high energy phosphates in organs stored in any of the solutions disclosed herein compared to the amount of HEPs produced using a storage solution lacking these ingredients. In other embodiments, the combination of citrulline and arginine synergistically produces a higher amount of nitrous oxide (NO) in organs stored in any of the solutions disclosed herein compared to the amount of NO produced using a storage solution lacking these ingredients. The synergism demonstrated by the combination of these components is both unexpected and surprising.

Accordingly, arginine (such as L-arginine) and citrulline (such as citrulline malate, for example, L-citrulline malate or salts thereof) can be present in the biological tissue and organ storage compositions described herein at concentrations between about 0.5 mM to 7 mM, such as any of about 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, or 7 mM, including all ranges and numbers falling within these values. In some embodiments, the organ storage composition does not comprise citrulline malate. In one embodiment, the concentration of arginine is about 1.074 g/L. In another embodiment, arginine is present at a concentration of about 5 mM. In some embodiments, the concentration of citrulline malate is about 0.175 g/l . . . In another embodiment, citrulline malate is present at a concentration of about 1 mM. Optionally, citrulline (such as L-citrulline) and malic acid can be added individually to the compositions in ranges of about 1-10 mM citrulline (such as any of about 1 mM, 2 mM, 3 mM, 4 mM, 5 mM, 6 mM, 7 mM, 8 mM, 9 mM, or 10 mM, including all ranges and numbers falling within these values) and about 1-5 mM malic acid (such as any of about 1 mM, 2 mM, 3 mM, 4 mM, or 5 mM, including all ranges and numbers falling within these values), respectively. In some embodiments, the organ storage composition does not comprise citrulline or citrulline malate. In yet other embodiments, the organ storage composition comprises malic acid from about 0.001 to about 7 mM, such as any of about 0.001 mM, 1 mM, 2 mM, 3 mM, 4 mM, 5 mM, 6 mM or 7 mM. In further embodiments, the organ storage solution does not comprise malate or malic acid.

Another component useful for maintaining ATP levels is adenosine. Adenosine can be present in the compositions disclosed herein at concentrations between about 1-4 mM, such as any of about 1 mM, 1.5 mM, 2 mM, 2.5 mM, 3 mM, 3.5 mM, or 4 mM, including all ranges and numbers falling within these values. In one embodiment, the concentration of Adenosine is about 0.534 g/L. In another embodiment. Adenosine is present at a concentration of about 2 mM. Adenosine also changes the polarization of the heart for rapid arrest; facilitates dialation of coronary vessels facilitating distribution/perfusion of the heart (organ) with the organ storage solution during storage, thereby attenuating hypoxia/ischemia related injury. Adenosine also slows the rate of K⁺-induced membrane depolarization, and reduces K⁺-induced intracellular Ca²⁺ loading in ventricular myocytes. Without being bound to theory, such findings support the notion that adenosine plays a cardioprotective role in hyperkalemic cardioplegia or during surgery and/or organ harvest by facilitating gentle arrest; especially for use in high K⁺ scenario, thus preventing inherent injury to the heart induced by such action. Further, without being bound to theory, in addition to its role in high energy phosphate production and ionic homeostasis, magnesium also protects the heart from repeated ischemia and injury via preconditioning, especially during prolonged organ storage as well as protecting against postoperative ventricular arrythemias upon open heart surgery or transplant. Both high potassium and magnesium concentrations also protects against calcium accumulation in mitochondria and subsequent injury to the organ.

In some embodiments of the biological tissue and organ storage compositions disclosed herein, the composition solution contains creatine. In some embodiments, creatine is present in the form of creatine orotate and/or creatine monohydrate or salts thereof. The concentration of creatine in the biological tissue and organ storage compositions can be between about 2-5 mM, such as about 2 mM, about 3 mM, about 4 mM or about 5 mM, including all ranges and numbers falling within these values. In another embodiment, the biological tissue and organ storage compositions can contain 0.5 mM creatine orotate. In another embodiment, the biological tissue and organ storage compositions can contain 0.27 g/L creatine orotate. In other embodiments, the biological tissue and organ storage compositions can contain 2 mM creatine monohydrate or salts thereof. In another embodiment, the biological tissue and organ storage compositions can contain 0.3 g/L creatine monohydrate or salts thereof. In yet another embodiment, the biological tissue and organ storage compositions contains both 0.5 mM creatine orotate and 2 mM creatine monohydrate or salts thereof. Creatine orotate can be difficult to obtain. Hence, in some embodiments, this can be changed to 0.5 mM orotic acid and salts thereof (0.50-2.50 mM) and 2.50 mM creatine monohydrate or salts thereof (2.50-10 mM). Both ex vivo and in vivo experiments show a beneficial effect from Mg—Or (magnesium orotate) administration at the onset of reperfusion on myocardial function and IS. In vitro assays showed that Mg—Or significantly delayed mPTP (mitochondrial pore transition) opening after 1/R. Without being bound to theory, this suggests that Mg—Or administered at the very onset of reperfusion may preserve myocardial function and reduce IS. This beneficial effect may be related to a significant reduction of mPTP opening, a usual trigger of cardiac cell death via apoptosis following I/R.

In some embodiments of the biological tissue and organ storage compositions disclosed herein, the composition solution contains a buffer for intracellular acidity, such as carnosine (for example, L-carnosine). The concentration of carnosine in the biological tissue and organ storage compositions can be between about 5-15 mM, such as about 5 mM, about 6 mM, about 7 mM, about 8 mM, about 9 mM, about 10 mM, about 11 mM, about 12 mM, about 13 mM, about 14 mM, or about 15 mM, including all ranges and numbers falling within these values. In another embodiment, the biological tissue and organ storage compositions can contain 2.3 g/L L-carnosine.

In other embodiments of the biological tissue and organ storage compositions disclosed herein, the solution contains carnitine (for example, L-carnitine), which facilitates a decrease in myocardial lactate production, hence reducing acidity. The concentration of carnitine in the biological tissue and organ storage compositions can be between about 5-15 mM, such as about 5 mM, about 6 mM, about 7 mM, about 8 mM, about 9 mM, about 10 mM, about 11 mM, about 12 mM, about 13 mM, about 14 mM, or about 15 mM, including all ranges and numbers falling within these values. In another embodiment, the biological tissue and organ storage compositions can contain 2 g/L L-carnitine.

Dichloroacetate, if present in the biological tissue and organ storage compositions disclosed herein, can control acidity by lowering lactate levels in the preserved organ, and thus the solution. The concentration of dichloroacetate in the biological tissue and organ storage compositions can be between about 0.1-2.5 mM, such as about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, or about 2.5 mM, including all ranges and numbers falling within these values. In another embodiment, the biological tissue and organ storage compositions can contain 0.08 g/L dichloroacetate. In other embodiments, the biological tissue and organ storage compositions contains no dichloroacetate.

In further embodiments, the biological tissue and organ storage compositions disclosed herein can contain insulin. Insulin can be added after the other ingredients are mixed and/or just prior to use of the storage compositions disclosed herein. For example, insulin can be added minutes, e.g., 0.5, 1, 2, 5, minutes to hours. e.g., 0.5, 1, 2, 3, 4, or 5 hours prior to immersing an organ in the solution. In some embodiments, about 100 units/L are added to the biological tissue and organ storage compositions.

The biological tissue and organ storage compositions disclosed herein can be maintained at a neutral or slightly basic pH, such as about pH 7, 7.1, 7.2, 7.3, 7.4, 7.5, 7.6, or 7.7, including all ranges and numbers falling within these values. In one embodiment, the pH of biological tissue and organ storage compositions is 7. In another embodiment, the pH of the biological tissue and organ storage compositions is modulated using THAM (Tris-Hydroxymethyl Aminomethane).

In other embodiments, the organ storage composition comprises the following nominal or base ingredients as shown in Table I for activity of iSomah, combined in deionized, distilled, and/or bacteriostatic water:

TABLE I

| Component | Amount | |
|---|---|---|
| Distilled water | 1 L | |
| Potassium chloride | At least 20 mM | |
| | potassium ions in | |
| | combination with KPO₄* | |
| Potassium phosphate | At least 20 mM | |
| | potassium ions in | |
| | combination with KCl* | |
| Magnesium chloride | At least 37 mM | |
| | magnesium ions in | |
| | combination with MgSO₄ | |
| Magnesium sulfate | At least 37 mM | |
| | magnesium ions in | |
| | combination with MgCl* | |
| Sodium chloride | 100-115 | mM |
| Sodium bicarbonate | 5-15 | mM |
| Sodium phosphate | 10-40 | mM |
| Glutathione | 1.5-5.0 | mM |
| Ascorbic acid | 1.0-5 | mM |
| Adenosine | 2-5 | mM |

In some embodiments, the potassium phosphate salt for use in the non-limiting formulation shown in Table I can be potassium phosphate monohasic. In another embodiment, the magnesium chloride salt for use in the non-limiting formulation shown in Table I can be magnesium chloride hexahydrate. In other embodiments, the magnesium sulfate salt for use in the non-limiting formulation shown in Table I can be magnesium sulfate heptahydrate. In yet other embodiments, the sodium phosphate salt for use in the non-limiting formulation shown in Table I can be sodium phosphate dibasic heptahydrate. In some embodiments, the glutathione for use in the non-limiting formulation shown in Table I can be reduced glutathione. In another embodiment, the creatine for use in the non-limiting formulation shown in Table I can be creatine monohydrate or salts thereof. In other embodiments, the non-limiting formulation shown in Table I can further comprise one or more of arginine (for example, L-arginine) in concentrations of between about 2 to about 10 mM, such as any of about 2 mM, 3 mM, 4 mM, 5 mM, 6 mM, 7 mM, 8 mM, 9 mM or 10 mM, carnosine (for example, L-carnosine) in concentrations of between about 5 to about 10 mM, such as any of about 5 mM, 6 mM, 7 mM, 8 mM, 9 mM, or 10 mM (2.26 g/L for 10 mM), carnitine (for example, L-carnitine) in concentrations of between about 5 to about 10 mM, such as any of about 5 mM, 6 mM, 7 mM, 8 mM, 9 mM, or 10 mM (2.26 g/L for 10 mM), orotic acid, for example, in concentrations of about 0.5-2 mM, such as any of about 0.5, 1, 1.5, or 2 mM, or creatine (for example, creatine monohydrate or salts thereof), in concentrations of about 2-5 mM, such as any of about 2 mM, 3 mM, 4 mM, or 5 mM. In another embodiment, the non-limiting formulation shown in Table I can further comprise insulin at a concentration of 10 mg-100 mg/ml/Liter or 100-1000 Units/L. When insulin is included in the composition, it is optionally added just prior to use as an organ preservation solution.

In yet other embodiments, the non-limiting formulation shown in Table I can further comprise a sugar, such as, but not limited to, a six carbon sugar (e.g., allose, altrose, galactose, glucose (including D-glucose (a.k.a. dextrose) and L-glucose), gulose, idose, mannose, talose, fructose, psicose, sorbose, tagatose, fucose, fuculose, or rhamnose) or a five carbon sugar (e.g. arabinose, lyxose, ribose, xylose, ketopentoses, ribulose, or xylulose) in concentrations from between about 11 mM to about 25 mM, such as any of about 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM sugar.

In still other embodiments, the non-limiting formulation shown in Table I can optionally comprise 1-10 mM of citrulline (for example, L-citrulline) or a salt thereof in concentrations of between about 2 to about 10 mM, such as any of about 2 mM, 3 mM, 4 mM, 5 mM, 6 mM, 7 mM, 8 mM, 9 mM, or 10 mM. In another embodiment, the non-limiting formulation shown in Table II can optionally comprise about 0-10 mM malic acid, such as any of about 0 mM, 1 mM, 2 mM, 3 mM, 4 mM, 5 mM, 6 mM, 7 mM, 8 mM, 9 mM, or 10 mM. In another embodiment, the non-limiting formulation shown in Table I can optionally comprise citrulline malate (such as L-citrulline malate) instead of malic acid and/or citrulline in concentrations of about 0 mM to about 10 mM or about 2 mM to about 7 mM, such as any of about 0 mM, 1 mM, 2 mM, 3 mM, 4 mM, 5 mM, 6 mM, 7 mM, 8 mM, 9 mM or 10 mM citrulline malate.

In other embodiments, and as a non-limiting example of iSomah, the biological tissue and organ storage compositions comprise the following ingredients combined in deionized and/or bacteriostatic water as shown in Table II:

TABLE II

| Component | Amount | |
|---|---|---|
| Distilled water | 1 | L |
| Potassium chloride | 4-65 | mM |
| Potassium phosphate (monobasic) | 0.44-10 | mM |
| Magnesium chloride (hexahydrate) | 0.5-65 | mM |
| Magnesium sulfate (heptahydrate) | 0.5-1.5 | mM |
| Sodium chloride | 80-135 | mM |
| Sodium bicarbonate | 2-25 | mM |
| Sodium phosphate (dibasic; heptahydrate) | 0.15-30 | mM |
| Calcium chloride | 0-1.5 | mM |
| D-Glucose | 5-25 | mM |
| Glutathione (reduced) | 0.5-3 | mM |
| Asorbic acid | 0.5-3 | mM |
| L-Arginine | 0.5 mM to 7 mM | |
| Malic acid | 0 mM to 7 mM | |
| | (optional) | |
| Adenosine | 1-4 | mM |
| orotic acid | 0.5-2 | mM |
| Creatine monohydrate | 2-5 | mM |
| L carnosine | 5-15 | mM |
| L-carnitine | 5-15 | mM |
| Dichloroacetate | 0.1-2.5 | mM |

| | |
|---|---|
| Insulin | 10 mg-100 mg/ml/Liter |
| | or 100-1000 Units/L |

In some embodiments, insulin is added after the other ingredients are mixed and/or just prior to use of the storage compositions. For example, insulin can be added minutes, e.g., 0.5, 1, 2, 5, minutes to hours, e.g., 0.5, 1, 2, 3, 4, or 5 hours prior to immersing an organ in the solution.

C. Cardioplegia Solutions

Cardioplegia solutions for arresting hearts during open heart surgery or for donor hearts for transplant are also provided herein. In one embodiment, a cardioplegia solution of the present invention can comprise a physiological salt solution containing at least 20 mM potassium ions (such as any of about 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM or more potassium ions, including all values and ranges falling in between these numbers) as well as one or more of a sugar (for example, ribose, glucose or dextrose), glutathione, ascorbic acid, arginine, citrulline (such as citrulline malate), adenosine, creatine (such as creatine orotate or creatine monohydrate or salts thereof), orotic acid, carnosine (such as L-carnosine), carnitine (such as L-camitine), and/or dichloroacetate. Cardioplegia solutions containing at least 20 mM potassium ions can be used to arrest hearts between about 4-10° C. (such as any of about 4° C., 5° C. 6° C. 7° C., 8° C., 9° C. or 10° C.).

In another embodiment, the cardioplegia solution of the present invention can comprise a physiological salt solution containing at least 20 mM potassium ions (such as any of about 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM or more potassium ions, including all values and ranges falling in between these numbers) and at least 37 mM magnesium ions (such as any of about 37 mM, 38 mM, 39 mM, 40 mM, 41 mM, 42 mM, 43 mM, 44 mM, 45 mM, 50 mM, 55 mM, 60 mM, 65 mM, 70 mM, 75 mM, 80 mM, 85 mM, 90 mM, 95 mM, or 100 mM or more magnesium ions, including all values and ranges falling in between these numbers) as well as one or more of a sugar (for example, ribose, glucose or dextrose), glutathione, ascorbic acid, arginine, citrulline (such as citrulline malate), adenosine, creatine (such as creatine orotate or creatine monohydrate or salts thereof), carnosine (such as L-carnosine), orotic acid, carnitine (such as L-carnitine), and/or dichloroacetate. Cardioplegia solutions containing at least 20 mM potassium ions and at least 37 mM magnesium ions can be used to arrest hearts between about 10-25° C., such as any of about 10° C., 11° C., 12° C., 13° C., 14° C., 15° C., 16° C., 17° C. 18° C., 19° C. 20° C., 21° C., 22° C., 23° C., 24° C., or 25° C. In yet other embodiments, the cardioplegia solution of the present invention can comprise a physiological salt solution containing at least 25 mM potassium ions (such as any of about 25 mM, 26 mM, 27 mM, 28 mM, 29 mM, 30 mM, 31 mM, 32 mM, 33 mM, 34 mM, 35 mM, 45 mM, 46 mM, 47 mM, 48 mM, 49 mM, 50 mM, 55 mM, 60 mM, 65 mM, 70 mM, 75 mM, 80 mM, 85 mM, 90 mM, 95 mM, 100 mM, 105, 110, 115, 120, 125 or more potassium ions, including all values and ranges falling in between these numbers) and at least 37 mM magnesium ions (such as any of about 37 mM, 38 mM, 39 mM, 40 mM, 41 mM, 42 mM, 43 mM, 44 mM, 45 mM, 50 mM, 55 mM, 60 mM, 65 mM, 70 mM, 75 mM, 80 mM, 85 mM, 90 mM, 95 mM, or 100 mM or more magnesium ions, including all values and ranges falling in between these numbers) as well as one or more of a sugar (for example, ribose, glucose or dextrose), glutathione, ascorbic acid, arginine, citrulline (such as citrulline malate), adenosine, creatine (such as creatine orotate or creatine monohydrate or salts thereof), orotic acid, carnosine (such as L-carnosine), carnitine (such as L-carnitine), and/or dichloroacetate. Cardioplegia solutions containing at least 25 mM potassium ions and at least 37 mM magnesium ions can be used to arrest hearts between about 25-37° C., such as any of about 25° C., 26° C., 27° C., 28° C., 29° C., 30° C., 31° C., 32° C., 33° C., 34° C. 35° C., 36° C., or 37° C. Other embodiments of cardioplegia solutions for arresting hearts during open heart surgery or for donor hearts for transplant comprising: a physiological salt solution containing between about 4-65 mM potassium ions (such as any of about 4 mM, 5 mM, 6 mM, 7, 8 mM, 9 mM, 10 mM, 15 mM, 20 mM, 25 mM, 30 mM, 35 mM, 40 mM, 45 mM, 50 mM, 55 mM, 60 mM, 61 mM, 62 mM, 63 mM, 64 mM, or 65 mM potassium ions, including all values and ranges falling in between these numbers) and between about 1.5-45 mM magnesium ions (such as any of about 1.5 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 41 mM, 42 mM, 43 mM, 44 mM, or 45 mM magnesium ions, including all values and ranges falling in between these numbers), as well as one or more of a sugar (for example, ribose, glucose or dextrose), glutathione, ascorbic acid, arginine, citrulline (such as citrulline malate), adenosine, creatine (such as creatine orotate or creatine monohydrate or salts thereof), carnosine (such as L-carnosine), orotic acid, carnitine (such as L-carnitine), and/or dichloroacetate. Cardioplegia solutions containing at least 45 mM potassium ions and at least 37 mM magnesium ions can be used to arrest hearts between about 4-37° C., such as any of about 4° C., 5° C., 6° C., 7° C., 8° C., 9° C., 10° C., 11° C., 12° C., 13° C., 14° C., 15° C., 16° C., 17° C., 18° C., 19° C. 20° C., 21° C., 22° C., 23° C., 24° C., 25° C., 26° C., 27° C., 28° C. 29° C., 30° C., 31° C., 32° C., 33° C., 34° C. 35° C., 36° C., or 37° C.

### III. Methods of the Invention

A. Methods for Storing Biological Tissue and Organs

Effective methods for storing biological tissue and organs using the compositions disclosed herein are also provided by the present invention. Biological tissue and organs can be stored in the solutions disclosed herein at ambient temperatures (for example, 10-21=4° C.). According to the methods provided herein, biological tissue and organs may be stored in the disclosed solutions for 24-72 hours, such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 or more hours, without significant accumulation of storage edema, free radical damage, an/or cellular/tissue damage commonly observed when organs are stored at temperatures at or near freezing.

Maintenance of high energy phosphate concentrations (such as, ATP) in biological tissue and organs during extended storage is important for the health of tissues and organs once they are transplanted into a donor (or resuscitated in the case of heart transplantation). Biological tissues and organs stored in any of the solutions disclosed herein according to the methods disclosed herein exhibit significantly more high energy phosphates (such as any of about 10%, 15%, 20%, 25%, 30%, 35%, 40%, 45%, 50%, 55%, 60%, 65%, 70%, or 75% more high energy phosphates including all ranges and numbers falling within these percentages) compared to biological tissue and organs that are not stored in the solutions disclosed herein.

Biological tissue and organs stored for prolonged periods of time exhibit significant increases in lactate production, which can negatively affect the pH of the storage media leading to increased tissue and cellular damage. Biological tissue and organs stored in any of the solutions disclosed herein according to the methods disclosed herein exhibit significantly less lactate production (such as any of about 10%, 15%, 20%, 25%, 30%, 35%, 40%, 45%, 50%, 55%, 60%, 65%, 70%, or 75%, less lactate production, including all ranges and numbers falling within these percentages) compared to biological tissue and organs that are not stored in the solutions disclosed herein.

When hearts are stored extracorporeally, coronary blood flow is often obstructed or decreased following storage and resuscitation. Hearts stored in any of the solutions disclosed herein according to the methods disclosed herein exhibit significantly higher levels of coronary blood flow (such as any of about 10%, 15%, 20%, 25%, 30%, 35%, 40%, 45%, 50%, 55%, 60%, 65%, 70%, or 75%, more coronary blood flow, including all ranges and numbers falling within these percentages) compared to hearts that are not stored in the solutions disclosed herein.

Additionally, when hearts are stored extracorporeally one or inmore of percent fractional area change, ejection fraction, and/or stroke volume as measured by epicardial 2-dimensional (2D) echocardiography can be decreased following storage and/or resuscitation. Hearts stored in any of the solutions disclosed herein according to the methods disclosed herein exhibit significantly higher levels of percent fractional area change, ejection fraction, and/or stroke volume (such as any of about 10%, 15%, 20%, 25%, 30%, 35%, 40%, 45%, 50%, 55%, 60%, 65%, 70%, or 75%, more percent fractional area change, ejection fraction, and/or stroke volume including all ranges and numbers falling within these percentages) compared to hearts that are not stored in the solutions disclosed herein.

B. Methods for Producing Biological Tissue and Organ Storage Compositions

Provided herein are methods for producing a composition for preserving biological tissue and organs, such as any of the compositions disclosed herein. The methods encompass mixing one or more of the ingredients described above at the indicated concentrations in distilled, deionized, and/or bacteriostatic water. In a further embodiment, and as a non-limiting example of iSomah, the method encompasses mixing one or more of the ingredients shown in the Table III below in distilled, deionized, and/or bacteriostatic water.

TABLE III

| Component | mM |
|---|---|
| Potassium Chloride | 7.00 |
| Potassium phosphate (monobasic) | 0.44 |
| Magnesium chloride (hexahydrate) | 0.5 |
| Magnesium sulfate (heptahydrate) | 0.503 |
| Sodium chloride | 125.00 |
| Sodium bicarbonate | 5.00 |
| Sodium phosphate (dibasic; heptahydrate) | 0.19 |
| D-Glucose | 11.00 |
| Glutathione (reduced) | 1.50 |
| Ascorbic acid | 1.00 |
| L-Arginine | 5.00 |
| L-Citrulline malate | 1.00 |
| Adenosine | 2.00 |
| Creatine orotate | 0.50 |
| Creatine monohydrate | 2.00 |
| L carnosine | 10.00 |
| L-carnitine | 10.00 |
| Dichloroacetate | 0.5 |
| Insulin | 100 units/L |

In some embodiments, insulin is added after the other ingredients are mixed and/or just prior to use of the storage compositions. For example, insulin can be added minutes, e.g., 0.5, 1, 2, 5, minutes to hours, e.g., 0.5, 1, 2, 3, 4, or 5 hours prior to immersing a biological tissue or organ in the solution.

In one embodiment of the methods disclosed herein, rather than adding citrulline malate (such as, L-citrulline malate) to the composition, 1-10 mM (such as any of about 1 mM, 2 mM, 3 mM, 4 mM, 5 mM, 6 mM, 7 mM, 8 mM, 9 mM, or 10 mM, including all ranges and numbers falling within these values) of citrulline (such as, L-citrulline) can be added along with 1-5 mM (such as any of about 1 mM, 2 mM, 3 mM, 4 mM, or 5 mM, including all ranges and numbers falling within these values) of malic acid, respectively.

In other embodiments, and as a non-limiting example of iSomah, the method encompasses mixing one or more of the ingredients shown in the Table IV below in distilled, deionized, and/or bacteriostatic water.

TABLE IV

| Component | mM |
|---|---|
| Potassium Chloride | 7.00 |
| Potassium phosphate | 0.44 |
| Calcium chloride | 1.30 |
| Magnesium chloride | 0.5 |
| Magnesium sulfate | 0.503 |
| Sodium chloride | 125.00 |
| Sodium bicarbonate | 5.00 |
| Sodium phosphate | 0.19 |
| D-Glucose | 11.00 |
| Glutathione | 1.50 |
| Asorbic acid | 1.00 |
| Arginine | 5.00 |
| Citrulline | 5.00 |
| Malic acid | 1.00 |
| Adenosine | 2.00 |
| Orotic acid | 0.50 |
| Creatine monohydrate | 2.50 |
| carnosine | 10.00 |
| carnitine | 10.00 |
| Dichloroacetate | 0.5 |
| Insulin | 100 units/L |

In some embodiments, insulin is added after the other ingredients are mixed and/or just prior to use of the storage compositions. For example, insulin can be added minutes, e.g., 0.5, 1, 2, 5, minutes to hours, e.g., 0.5, 1, 2, 3, 4, or 5 hours prior to immersing a biological tissue or organ in the solution.

In some embodiments, the potassium phosphate salt for use in producing the non-limiting formulation shown in Table IV can be potassium phosphate monobasic. In another embodiment, the magnesium chloride salt for use in the non-limiting formulation shown in Table IV can be magnesium chloride hexahydrate. In other embodiments, the magnesium sulfate salt for use in the non-limiting formulation shown in Table IV can be magnesium sulfate heptahydrate. In yet other embodiments, the sodium phosphate salt for use in the non-limiting formulation shown in Table IV can be sodium phosphate dibasic heptahydrate. In some embodiments, the glutathione for use in the non-limiting formulation shown in Table IV can be reduced glutathione. In another embodiment, the creatine for use in the non-limiting formulation shown in Table IV can be creatine monohydrate or salts thereof. In another embodiment, the arginine for use in the non-limiting formulation shown in Table IV can be L-arginine. In another embodiment, the carnosine for use in the non-limiting formulation shown in Table IV can be L-carnosine. In another embodiment, the carnitine for use in the non-limiting formulation shown in Table IV can be L-carnitine.

The method can also include a step of adjusting the pH of the solution to a neutral or slightly basic level, such as about pH 7, 7.1, 7.2, 7.3, 7.4, 7.5, 7.6, or 7.7, including all ranges and numbers falling within these values. In one embodiment, the pH of the biological tissue and organ storage composition is adjusted to 7.5.

In preferred embodiments, and as non-limiting examples of iSomah, methods for producing an organ preservation composition encompass mixing one or more of the following ingredients at the indicated concentrations in Table V or Table Va in distilled, deionized, and/or bacteriostatic water.

TABLE V

| Component | mM |
|---|---|
| Potassium Chloride | 20 |
| Potassium phosphate | 0.44 |
| Calcium chloride | 1.30 |
| Magnesium chloride | 37 |
| Magnesium sulfate | 0.5 |
| Sodium chloride | 100.00 |
| Sodium bicarbonate | 5.00 |
| Sodium phosphate | 30.00 |
| D-Glucose | 11.00 |
| Dichloroacetate | 0.50 (optional) |
| Malic acid | 1.0 |
| Glutathione | 1.50 |
| Asorbic acid | 1.00 |
| Adenosine | 2.00 |
| Orotic acid | 0.50 |
| Creatine | 2.50 |
| carnosine | 10.00 |
| carnitine | 10.00 |
| Arginine | 5.00 |
| Citrulline | 5.00 |
| Insulin | 100 units/L |

wherein insulin is added just prior to use.

TABLE Va

| Component | mM |
|---|---|
| Potassium Chloride | 20 |
| Potassium phosphate | 0.44 |
| Calcium chloride | 1.30 |
| Magnesium chloride | 37 |
| Magnesium sulfate | 0.5 |
| Sodium chloride | 100.00 |
| Sodium bicarbonate | 5.00 |
| Sodium phosphate | 30.00 |
| D-Glucose | 11.00 |
| Dichloroacetate | 0.50 (optional) |
| Malic acid | 1.0 |
| Glutathione | 1.50 |
| Asorbic acid | 1.00 |
| Adenosine | 2.00 |
| Orotic acid | 0.50 |
| Creatine | 2.50 |
| carnosine | 10.00 |
| carnitine | 10.00 |
| Arginine | 5.00 |
| Citrulline | 5.00 |

In some embodiments, the potassium phosphate salt for use in the non-limiting formulation shown in Table V or Table Va can be potassium phosphate monobasic. In another embodiment, the magnesium chloride salt for use in the non-limiting formulation shown in Table V or Table Va can be magnesium chloride hexahydrate. In other embodiments, the magnesium sulfate salt for use in the non-limiting formulation shown in Table V or Table Va can be magnesium sulfate heptahydrate. In yet other embodiments, the sodium phosphate salt for use in the non-limiting formulation shown in Table V or Table Va can be sodium phosphate dibasic heptahydrate. In some embodiments, the glutathione for use in the non-limiting formulation shown in Table V or Table Va can be reduced glutathione.

In further embodiments, the non-limiting formulation shown in Table V or Table Va can further comprise one or more of arginine (for example, L-arginine) in concentrations of between about 2 to about 10 mM, such as any of about 2 mM, 3 mM, 4 mM, 5 mM, 6 mM, 7 mM, 8 mM, 9 mM or 10 mM, carnosine (for example, L-carnosine) in concentrations of between about 5 to about 10 mM, such as any of about 5 mM, 6 mM, 7 mM, 8 mM, 9 mM, or 10 mM (2.26 g/L for 10 mM), carnitine (for example, L-carnitine) in concentrations of between about 5 to about 10 mM, such as any of about 5 mM, 6 mM, 7 mM, 8 mM, 9 mM, or 10 mM (2.26 g/L for 10 mM), orotic acid, for example, in concentrations of about 0.5-2 mM, such as any of about 0.5, 1, 1.5, or 2 mM, or creatine (for example, creatine monohydrate or salts thereof), in concentrations of about 2-5 mM, such as any of about 2 mM, 3 mM, 4 mM, or 5 mM. In another embodiment, the non-limiting formulation shown in Table V can further comprise insulin at a concentration of 10 mg-100 mg/ml/Liter or 100-1000 Units/L. When insulin is included in the composition, it is optionally added just prior to use as an organ preservation solution.

In yet other embodiments, the non-limiting formulation shown in Table V or Table Va can further comprise a sugar, such as, but not limited to, a six carbon sugar (e.g., allose, altrose, galactose, glucose (including D-glucose (a.k.a. dextrose) and L-glucose), gulose, idose, mannose, talose, fructose, psicose, sorbose, tagatose, fucose, fuculose, or rhamnose) or a five carbon sugar (e.g. arabinose, lyxose, ribose, xylose, ketopentoses, ribulose, or xylulose) in concentrations from between about 11 mM to about 25 mM, such as any of about 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM of sugar.

In still other embodiments, the non-limiting formulation shown in Table V or Table Va can optionally comprise 1-10 mM of citrulline (for example, L-citrulline) or a salt thereof in concentrations of between about 2 to about 10 mM, such as any of about 2 mM, 3 mM, 4 mM, 5 mM, 6 mM, 7 mM, 8 mM, 9 mM, or 10 mM. In another embodiment, the non-limiting formulation shown in Table V or Table Va can optionally comprise citrulline malate (such as L-citrulline malate) instead of malic acid and/or citrulline in concentrations of about 0 mM to about 10 mM or about 2 mM to about 7 mM, such as any of about 0 mM, 1 mM, 2 mM, 3 mM, 4 mM, 5 mM, 6 mM, 7 mM, 8 mM, 9 mM or 10 mM citrulline malate.

In another embodiment, and as a non-limiting example of iSomah, methods for producing an organ preservation composition encompass mixing one or more of the following ingredients at the indicated concentrations in Table VI in distilled, deionized, and/or bacteriostatic water.

TABLE VI

| Component | Amount | |
|---|---|---|
| Distilled water | 1 | L |
| Potassium chloride | 4-65 | mM |
| Potassium phosphate | 0.44-10 | mM |
| Magnesium chloride | 0.5-65 | mM |
| Magnesium sulfate | 0.5-1.5 | mM |
| Sodium chloride | 80-135 | mM |
| Sodium bicarbonate | 2-25 | mM |
| Sodium phosphate | 0.15-30 | mM |
| Calcium chloride | 0-1.5 | mM |
| D-Glucose | 5-25 | mM |
| Glutathione | 0.5-3 | mM |
| Asorbic acid | 0.5-3 | mM |
| Arginine | 0.5 mM to 7 mM | |
| Malic acid | 0 mM to 7 mM | |
| | (optional) | |
| Adenosine | 1-4 | mM |
| Orotic acid | 0.5-2 | mM |
| Creatine | 2-5 | mM |
| carnosine | 5-15 | mM |
| carnitine | 5-15 | mM |
| Dichloroacetate | 0.1-2.5 | mM |

| | |
|---|---|
| Insulin | 10 mg-100 mg/ml/Liter |
| | or 100-1000 Units/L |

wherein insulin is added just prior to use.

### IV. Kits

The compositions for making the biological tissue and organ storage/resuscitation solutions disclosed herein are optionally packaged in a kit with the ingredients listed below or multiples thereof in amounts necessary to scale up to make 2, 3, 5, 10, 20 times the amount of solution. An exemplary kit contains one or more of glutathione, ascorbic acid, adenosine, potassium chloride, potassium phosphate magnesium chloride, magnesium sulfate, sodium chloride, sodium bicarbonate, sodium phosphate, a sugar (such as ribose, glucose or dextrose), arginine, citrulline malate, adenosine, orotic acid, creatine, and dichloroacetate (for example, one or more of about 2.76 g/L Potassium Chloride, 0.06 g/L Potassium phosphate (monobasic), 7.47 g/L Magnesium chloride (hexahydrate), 0.123 g/L Magnesium sulfate (heptahydrate), 7.30 g/L Sodium chloride, 0.35 g/L Sodium bicarbonate, 0.05 g/L Sodium phosphate (dibasic; heptahydrate), 1.98 g/L D-Glucose, 0.462 g/L Glutathione (reduced), 0.18 g/L Asorbic acid, 0.21 g/L Arginine, 0.15 g/L L-Citrulline malate, 0.27 g/L Adenosine, 0.27 g/L Creatine orotate, orotic acid 0.373 g/L, Creatine monohydrate or salts thereof, 2.3 g/L L-carnosine, 2.0 g/L L-carnitine, 0.08 g/L Dichloroacetate, and 100 units/L insulin. The kit may optionally also contain citrulline (such as L-citrulline), and malic acid.

These ingredients can be packaged together with instructions for use and are mixed in 0.01-2.0 L of distilled water. The kit may also contain solutions of means for adjusting the pH of the combined biological tissue and organ preservation/storage solution (e.g. THAM). The kit can be packaged or sold with or without the sterile and/or deionized water component.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### EXAMPLES

### Example 1

In the following Examples, tables are designated using Arabic numbers (e.g. Table 1, Table 2, Table 3, etc.).

In the last 40 years, notable progress has been made in the preservation of solid organs such as livers and kidneys, allowing for extracorporeal preservation for up to several hours. Unfortunately, the same cannot be ascribed to preservation of hearts, which, even after decades of arduous effort, can be stored ex vivo only for 4 to 6 hours (Churchill T A. Organ preservation for transplantation. In: Storey K B, editor. Functional metabolism: regulation and adaptation. John Wiley-Liss; 2004; 529-55). Major advances include the conceptual development of solutions, such as Celsior and University of Wisconsin solution (UWS; Table 1-1), the formulation of which was based on the prevention of edema during “hypothermic storage at 4° C.”—a concept deeply ingrained in the philosophy of organ preservation that allows for only short-term ex vivo storage. This theory likely exists because, at such extremely low temperatures, there is tissue/cellular damage, rendered irreversible with time (Devillard et al., Mol Cell Biochem 2008; 307:149-57; Belzer et al., Transplantation 1988; 45:673-6; Southard et al., Annu Rev Med 1995; 46:235-47). Organ edema is prevented by the addition of cellular impermeants such as mannitol and lactobionic acid to Celsior, and lactobionic acid, raffinose and hydroxyethyl starch to UWS. Because cellular adenosine triphosphate (ATP) consumption/synthesis is essentially very low at 41° C., there is not much stress on maintenance of cardiomyocyte high-energy phosphates (HEP; such as ATP and creatine phosphate) in the formulation of Celsior and UWS, except for addition of adenosine in UWS.

In this Example it is shown that by enhancing high energy phosphate (HEP) synthesis, extracorporeal storage of hearts at higher temperatures is possible. Unlike Celsior, porcine hearts stored at 4° C. in Somah demonstrate robust maintenance of organ viability after 4-hour storage (Thatte et al., Circulation 2009; 120:1704-13). In addition, hearts stored at 21° C. in Somah are viably superior to those stored at extreme hypothermia (4° C.) (Lowalekar et al., Transplant Proc 2013; 45:3192-7) and have demonstrated far superior functional revival upon extracorporeal reperfusion (Lowalekar et al., Am J Transpl 2014 October; 14(10):2253-62).

Materials and Methods

Heart procurement surgery. Female Yorkshire swine (45 to 54 kg) were used as approved by the animal studies committee in strict accordance with established humane policies. Hearts were extracted as described elsewhere (Thatte et al., Circulation 2009; 120:1704-13) and femoral blood was collected for ex vivo experiments before clamping the aorta at a systolic pressure of <40 mm Hg. Then 1.000-ml cardioplegia at 21° C., with either Somah (20 mmol/liter K⁺). Celsior (20 mmol/liter K⁺) or UWS (innate 140 mM K⁺), was infused at 75 to 100 mm Hg; hearts were excised and stored in Somah, Celsior or UWS for 5 hours at 21±2° C. (ambient temperature).

Extracorporeal heart storage. Hearts were placed inside ziplock bags (with 2 liters of Somah, Celsior or UWS) in a waterjacketed bath at 21±2° C. Because hearts in Somah showed slow contractile movements during storage, Somah's plegia potential was enhanced by supplemental K⁺ (total 20 mmol/liter) and Mg²⁺ (37 mmol/liter) (Fukuhiro et al., Circulation 2000; 102(III):319-25). Hearts were weighed beforehand and after 5 hours. Punch biopsies (2×4 mm) were taken from the left ventricular (LV) posterior wall, before and at the end of storage, for HEP assays.

ATP and creatine phosphate assay. Cardiac tissue HEP were measured as described elsewhere (Devillard et al., Mol Cell Bioclhem 2008; 307:149-57; Bessho et al., Anal Biochem 1991; 192:117-24). Briefly, tissue was suspended in cold perchloric acid and homogenized. Homogenate was centrifuged at 0° C., the pellet was dissolved in NaOH for protein quantitation, and the supernatant was neutralized with cold KHCO₃ and again centrifuged before HEP (ATP+CP) measurement using a bioluminescent kit and protocol (Promega GloMax-Multi+Detection System; Sigma-Aldrich).

TABLE 1-1
Composition of Somah, Celsior, and UW solutions
Somah (pH 7.5)

| | |
|---|---|
| Potassium phosphate (monobasic) | 0.44 |
| Potassium chloride | 7.00 |
| Sodium chloride | 125.00 |
| Sodium bicarbonate | 5.00 |
| Calcium chloride | 1.30 |
| Sodium phosphate (dibasic, heptahydrate) | 0.19 |
| Magnesium chloride (hexahydrate) | 0.50 |
| Magnesium sulfate (heptahydrate) | 0.50 |
| D-glucose | 11.00 |
| Glutathione (reduced) | 1.50 |
| Ascorbic acid | 1.00 |
| L-arginine | 5.00 |
| L-citrulline malate | 1.00 |
| Adenosine | 2.00 |
| Creatine orotate | 0.50 |
| Creatine monohydrate | 2.00 |
| L-carnosine | 10.00 |
| L-carnitine | 10.00 |
| Dichloroacetate | 0.50 |
| Insulin | 100 units/liter |

Celsior (pH 7.3)

| | |
|---|---|
| Mannitol | 60.00 |
| Lactobionic acid | 80.00 |
| Glutamic acid | 20.00 |
| Histidine | 30.00 |
| Calcium chloride | 0.25 |
| Potassium chloride | 15.00 |
| Magnesium chloride | 2.64 |
| Sodium hydroxide | 100.00 |
| Glutathione SH | 3.00 |

UW solution (pH 7.4)

| | |
|---|---|
| Lactobionic acid | 105.00 |
| Potassium dihydrogen phosphate | 25.00 |
| Potassium hydroxide (56%) | 100.00 |
| Sodium hydroxide (40%) | 27.00 |
| Magnesium sulfate (heptahydrate) | 5.00 |
| Raffinose (pentahydrate) | 30.00 |
| Adenosine | 5.00 |
| Glutathione | 3.00 |
| Allopurinol | 1.00 |
| Hydroxyethyl starch (pentafraction) | 50 g/liter |
| Insulin | 40 units/liter |

^{a}Date expressed in millimoles per liter (mmol/liter), unless specified otherwise.

Preparation of heart for ex vivo resuscitation and functional studies. After separation from adjoining tissue and other vessels, the aorta, pulmonary veins (PVs) and pulmonary artery were cannulated using ½-to ⅜-in., ½-to ¼-in. and ½-to ⅜-in. tubing connectors, respectively, whereas vena cavae were ligated.

Preparation of blood for ex vivo studies. Systemically heparinized blood was collected intra-operatively, leukodepleted using a sterile leukocyte reduction filler (Pall Lukoguard R S), and stored at 41° C. Perfusate hematocrit was adjusted to 20% using Somah or Plasmalyte (+1.3 mmol/liter calcium) at a 1:1 ratio, to reduce viscous strain on the heart during extracorporeal perfusion. Perfusate pH, glucose, K⁺, Ca²⁺ and HCO₃⁻ were adjusted for swine blood levels (pH 7.5; 100 mg/dl; 3.7, 1.38 and 32 mmol/liter, respectively), using 10% dextrose, KCl, CaCl₂ and NaHCO₃, respectively.

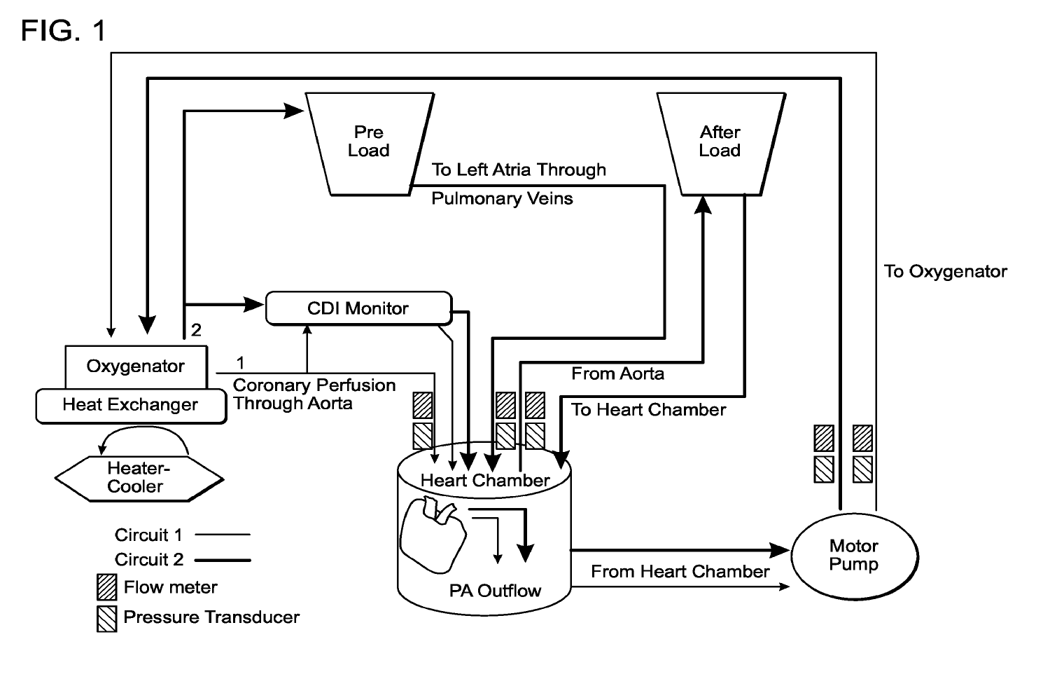

Somah device. A custom-built apparatus was used for extracorporeal reanimation of hearts (FIG. 1). A CDI monitor (Clinical Documentation Improvement Monitoring System 500; Terumo Cardiovascular Systems Corp., Ann Arbor, Mich.), was used for real-time monitoring of perfusate pH, temperature, PO₂, PCO₂, K⁺ and HCO₃⁻. These parameters were also analyzed in inflow/outflow samples using an iSTAT analyzer (Abaxis, Ltd., Union City, Calif.).

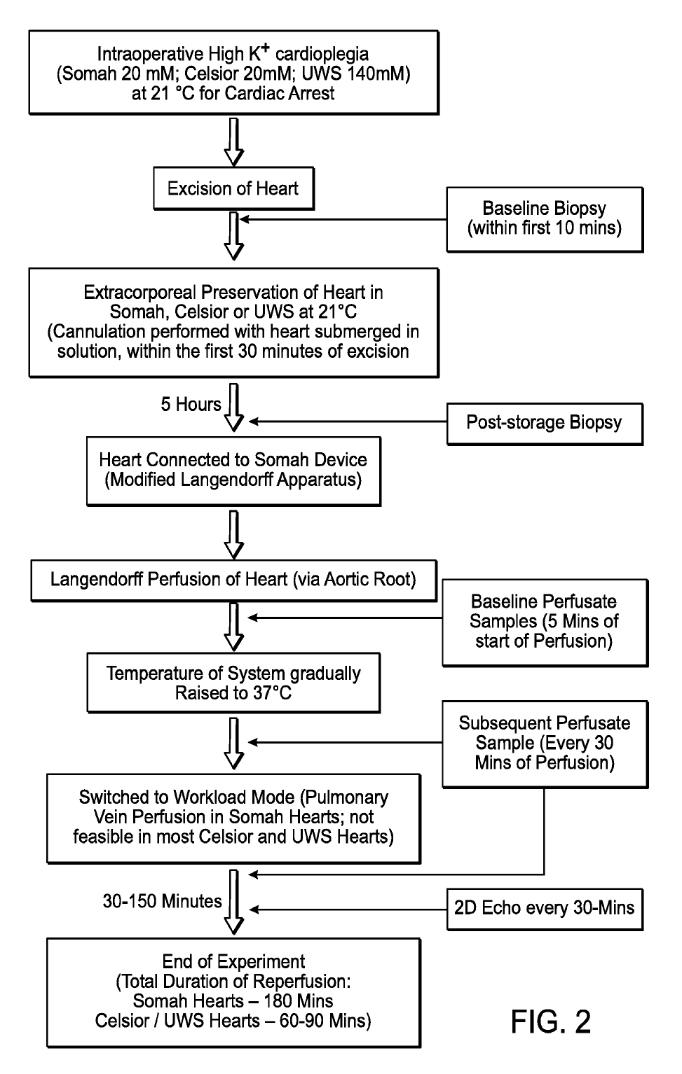

Ex vivo functional studies. UWS hearts were flushed with saline to clear excess potassium. Upon attachment to the Somah device, Somah and Celsior/UWS hearts were flushed at 40 to 60 mm Hg, with 1.5 liters of Somah (pH 7.5) or Plasma-Lyte A (pH 7.5; clinically used physiologic solution) solutions, respectively, followed by perfusate. Pressure flow data were acquired using HMI software. The system temperature was raised to 37° C. over 30 minutes. Average duration for post-perfusion assessment in Somah hearts was 180 minutes, in contrast to 60 and 120 minutes, respectively, for Celsior and UWS hearts due to development of myocardial contracture and/or poor performance, even after several minutes of reperfusion upon reaching 37° C. Electroconversion (40 to 50 J) and/or epinephrine (1:50.000 to 1:100,000 in Somah hearts, as per previous experience (Lowalekar et al., Am J Transpl 2014 October; 14(10):2253-62) or 1:10,000 for Celsior/UWS hearts as reported elsewhere (Hill et al., Am Thorac Surg 2005; 79:168-77)) were used if required. Inflow (aortic) and outflow (caval) samples were collected at the beginning and then every 30 minutes (FIG. 2). Epicardial 2-dimensional (2D) echocardiography was performed using a transesophageal echocardiography (TEE) probe for functional assessment.

Enzyme assays and blood chemistry. Blood creatine kinase (CK), troponin I (cTnI), lactate and gases (partial pressure of oxygen/partial pressure of carbon dioxide [PO₂/PCO₂]) were measured using analyzers (Vetscan VS2 and iSTAT; Abaxis), intra-operatively, during storage and at reperfusion. MVO₂ was calculated during reperfusion as described elsewhere (Klabunde R. Cardiac function. Cardiovascular physiology concepts. Philadelphia: Lippincott Williams & Wilkins; 84-8).

Epicardial echocardiography. Two-dimensional echocardiography data (short- and long-axis views for cardiac functional parameters, and LV septal/ventricular wall thicknesses) was acquired intra-operatively and ex vivo (epicardial) with a TEE probe (Cypress system; Acuson, Mountain View, Calif.) once the system temperature reached 37° C., 30 minutes after start of perfusion, and then analyzed with CYPRESS-VIEWER software. Three-lead ECG was recorded during 2D echocardiography using brass crocodile leads immersed in perfusate in the heart chamber. All Somah hearts sustained full workload (PV perfusion) and sinus rhythm, but not all Celsior and UWS hearts could tolerate the switch to full workload.

Statistical analyses. For each investigation, equal numbers of animals from each group were assigned for comparative analysis. Biopsies and other samples were timed, as shown in FIG. 2. Coronary and aortic flow/pressure data were acquired using HMI software. SIGMA-PLOT software was used for statistical analysis. For comparisons between groups, a Kruskal-Wallis 1-way analysis of variance (ANOVA) ranks test was performed, assuming non-parametric data. If normality and equal variance testing passed, further analysis was performed using a Holm-Sidak test or Dunn's test; otherwise, data were compared by 1-way ANOVA. Assuming nonparametric data, to determine significant alterations within the same group, the Mann-Whitney rank-sum test was performed. If normality and equal variance testing passed, Student's t-test was performed. p<0.05 was considered significant. All values are presented as mean±SEM.

Results

Intra-operative cardioplegia. All hearts received cardioplegia at 21° C. Cardiac arrest occurred immediately in the UWS group, likely due to very high K+. In contrast, it took 20 to 25 and 30 to 40 seconds in the Somah and Celsior groups, respectively, for complete cardiac arrest.

Gross morphology, heart weights and release of enzymes during storage. All stored hearts presented normal gross morphology without discoloration and were pliable with no signs of rigidity/stiffness. Heart weights were not altered during 5-hour storage, suggesting absence of gross edema. A minimal release of cardiac enzymes (CK/cTnI) was detected in all solutions during storage.

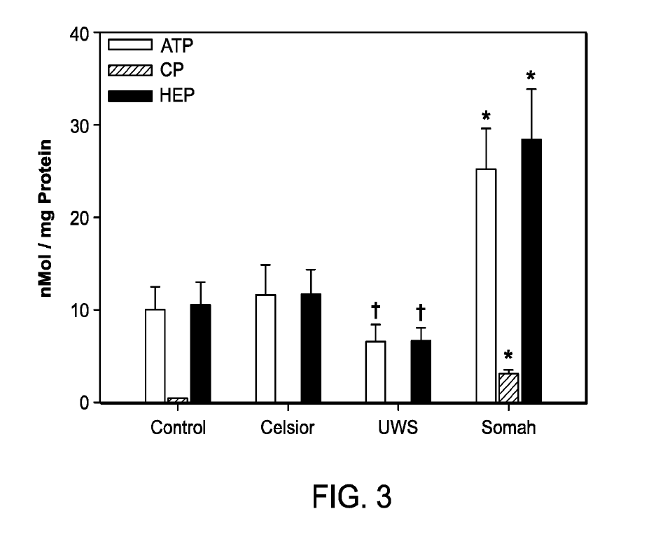

Cardiac tissue HEP levels during storage. After storage, HEP levels were significantly enhanced in Somah stored hearts (28.33±5.51; p<0.001), significantly decreased in UWS hearts (5.92±1.46; p<0.05), but remained unaltered in Celsior hearts (11.57±2.77), as compared with controls (9.95±2.52 nmol/liter per milligram protein) (FIG. 3), and there was a significantly greater build-up of HEP in Somah hearts than in the comparison groups (p<0.001).

Coronary flow upon reperfusion. Coronary flow upon initial perfusion at 21° C. was significantly greater in Somah hearts than in Celsior or UWS hearts at similar perfusion pressures (Table 2). Somah hearts, but not Celsior/UWS hearts, demonstrated slow contractions immediately upon initiation of reperfusion. With the increase in system temperature to 37° C., coronary flow increased significantly in Somah and UWS hearts but not in Celsior hearts, and was highest and nearly normal in Somah hearts. In the UWS group, there was a drop in coronary circulation pressure alter an initial abrupt rise.

TABLE 1-2
Alterations in coronary flow in Somah, Celsior, and UES group hearts, with rise in system
temperature of Somah Device.

| | P1-21° C. | F1-21° C. | P2-30° C. | F2-30° C. | P3-37° C. | F3-37° C. |
|---|---|---|---|---|---|---|
| Somah | 40 ± 1 | 386 ± 68^{a} | 44 ± 2 | 375 ± 62^{a} | 53 ± 4 | 579 ± 35^{b,c} |
| Celsior | 39 ± 1 | 272 ± 35 | 51 ± 7 | 199 ± 44 | 55 ± 5 | 289 ± 99 |
| UWS | 38 ± 3 | 163 ± 13 | 53 ± 6 | 195 ± 44 | 38 ± 3 | 443 ± 80^{b,c} |

P1, P2, P3: aortic root pressures at respective temperatures;
F1, F2, F3: coronary flows at respective temperature.
UWS, University of Wisconsin solution.
^{a}Significant difference from other groups at same temperature.
^{b}Significant difference from 21° C. in the same group.
^{c}Significant difference from Celsior group hearts.

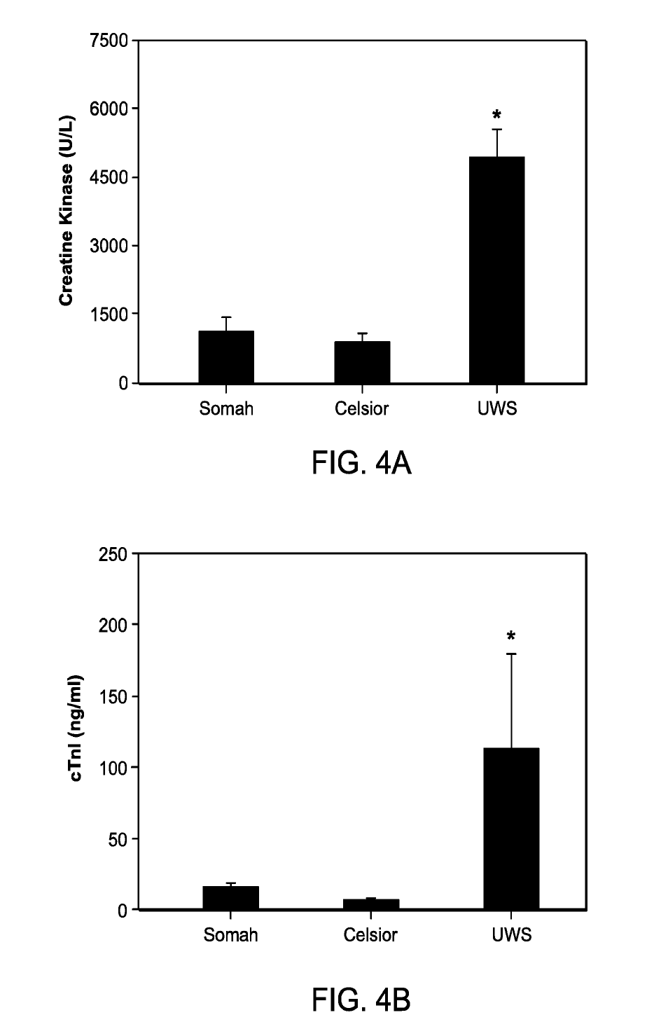

Release of enzymes upon reperfusion. Whereas CK and cTnI release in Somah and Celsior hearts was comparable after 30 minutes of reperfusion, there was a significantly greater release of both by UWS hearts (FIGS. 4A and B).

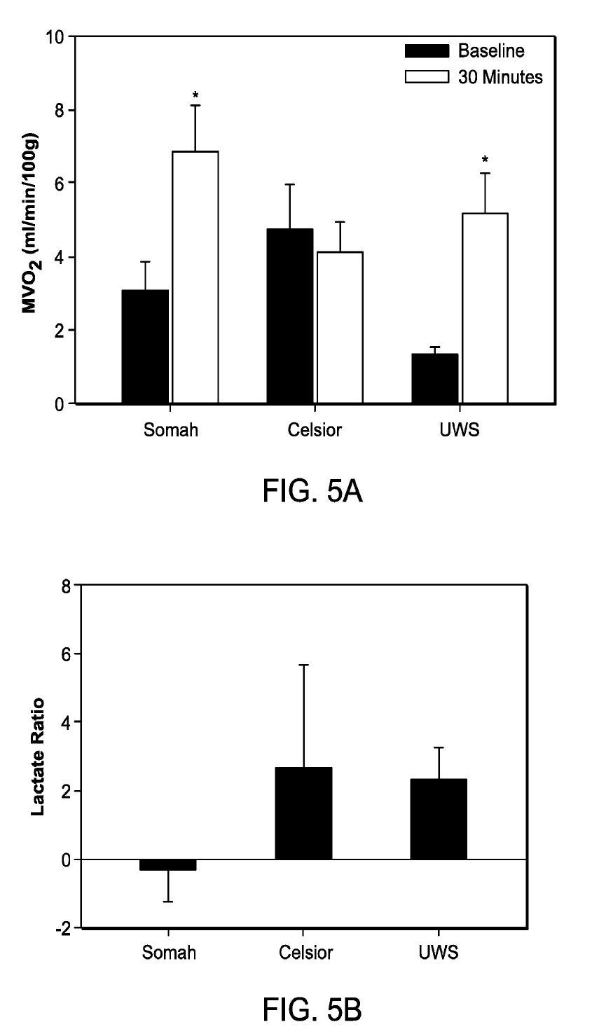

Metabolism in reperfused hearts. There was a rapid switch from anaerobic to aerobic metabolism within 30 minutes of reperfusion in Somah hearts, as suggested by the increased MVO₂ and lactate ratio reversal (FIGS. 5A and B). In contrast, although a positive lactate production (unturned lactate ratio) was evident in Celsior and UWS hearts, MVO₂ was unaltered in Celsior hearts during the same time period (FIGS. 5A and B).

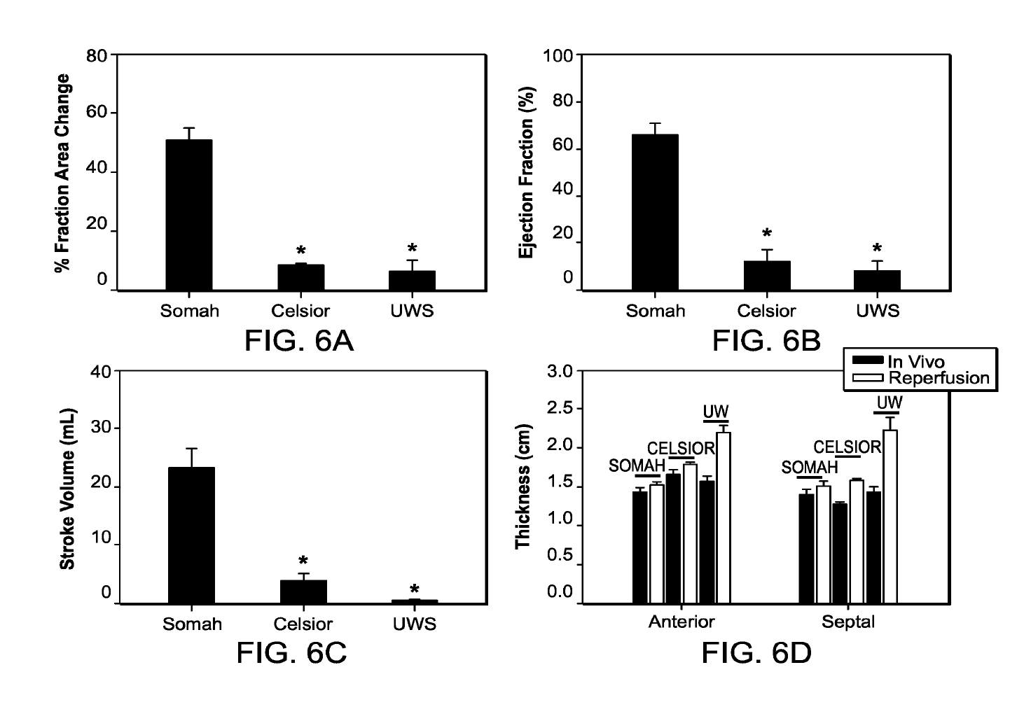

Functional revival upon reperfusion. Upon reperfusion, immediate spontaneous atrioventricular activity, complemented by rudimentary electrical activity (ECG), was apparent in Somah hearts with further augmentation of ventricular contraction as system temperature was raised to 37° C., and a single cardioversion established sinus rhythm. Somah hearts did not require inotropic support or further electroversions and remained pliabl throughout the investigation. In contrast. Celsior and UWS hearts demonstrated minimally visible spontaneous activity, not detectable electrically, and became progressively firm to the touch, beginning at the LV apex, eventually involving the entire LV and septal walls, and ultimately the RV and atria, suggesting possible initialization of ischemia-reperfusion injury (IRI). Accordingly, perfusate Ca²⁺ levels also rapidly dropped, suggesting an intracellular shift. Celsior/UWS hearts were not revertible despite several attempts at cardioversion and epinephrine infusion. Thus, functional 2D echocardiographic data could not be procured successfully in every Celsior/UWS heart and the cardiac functional data exemplified significantly lower performance in Celsior/UWS hearts (FIG. 6A-C). Moreover, post-perfusion LV anterior/septal wall thicknesses were greatly increased in UWS hearts (FIG. 6D).

In conclusion, Somah may be the key “solution” for improving prognosis in heart transplant patients. Robust metabolism, superior functional revival upon reanimation, decreased IRI-dependent damage and diminished requirements for stimulatory interventions that hearts stored in Somah at sub-normothermia are more likely to revert rapidly to robust functionality compared with other preservation solutions.

### Example 2

This Example examines whether recovery of post storage heart functionality is proportionally dependent on the maintenance of the organ's energy state and storage temperature and compares heart preservation in Celsior at 4° C. and in Somah at 4° C., 13° C., and 21° C., respectively.

Materials and Methods

Heart procurement surgery, extracorporeal heart storage, ATP and creatine phosphate assays, preparation of hearts for ex vivo resuscitation and functional studies, preparation of blood for ex vivo studies, the Somah device, and functional studies were performed as described in Example 1, above.

Heart weight and biopsies. Heart chambers were emptied prior to weighing at the start of storage and after 5 h. Cardiac punch biopsies (2-4 mm diameter) were taken using punch forceps within 15 min of heart excision (controls) and at the end of 5-h storage from the posterior wall of left ventricle (LV) for histopathology (HP; hematoxylin and eosin staining) and ultrastructure for Somah hearts and HEP assays for Somah and Celsior hearts.

Electron microscopy. Somah heart tissue was fixed in glutaraldehyde and processed for ultrastructure studies. Briefly, tissue taken for electron microscopy (EM) studies was immediately fixed in glutaraldehyde and stored at 4° C. After fixation, dehydration and embedding, 70-100 nm sections were cut using ultramicrotome, transferred to grid and examined under JEOL electron microscope (1200EX-80 kV; JEOL USA Inc., Peabody, Mass.) to identify any ultrastructural changes.

Enzyme assays and blood chemistry. Creatine kinase (CK), cardiac troponin-I (cTnI), lactate and gases (pO₂/pCO₂) were measured intra-operatively, and in Somah samples taken at 10 min, 2 h, and at end of 5-h heart storage using Vetscan VS2 or iStat (Abaxis Ltd, Union City, Calif.). Components of Celsior interfered with the assays; hence, storage samples could not be assayed. Inflow (aortic) and outflow (caval) samples were collected for enzyme assays at 5 min and 90 min after start of perfusate perfusion, and at 60 min (baseline) and 90 min (peak performance) for assessment of myocardial O₂ consumption (MVO₂) and lactate levels, using Vetscan VS2 or i-Stat System. MVO₂ was calculated. The Vetscan CK assay values obtained during in vitro reperfusion were specific for heart in these isolated heart studies.

Epicardial echocardiography. TEE probe was used for 2D Echo evaluation of cardiac function intraoperatively and ex vivo, using Acuson Cypress system (Acuson, Mountain View, Calif.) and images analyzed using Cypress viewer software. During ex vivo experiments, heart was connected to Somah Device and suspended in a chamber containing 2 L perfusate covering two-thirds of hearts surface. An electrocardiogram was recorded from beginning and 2D Echo acquisition was begun approximately 45-60 min after perfusion, if and when good cardiac contractions were observed, and repeated at 30-min intervals. Probe was placed in direct contact with heart, and angle of the probe and direction of pulse adjusted to obtain short-axis and long-axis views to assess for cardiac functional parameters and ventricular and septal wall thicknesses.

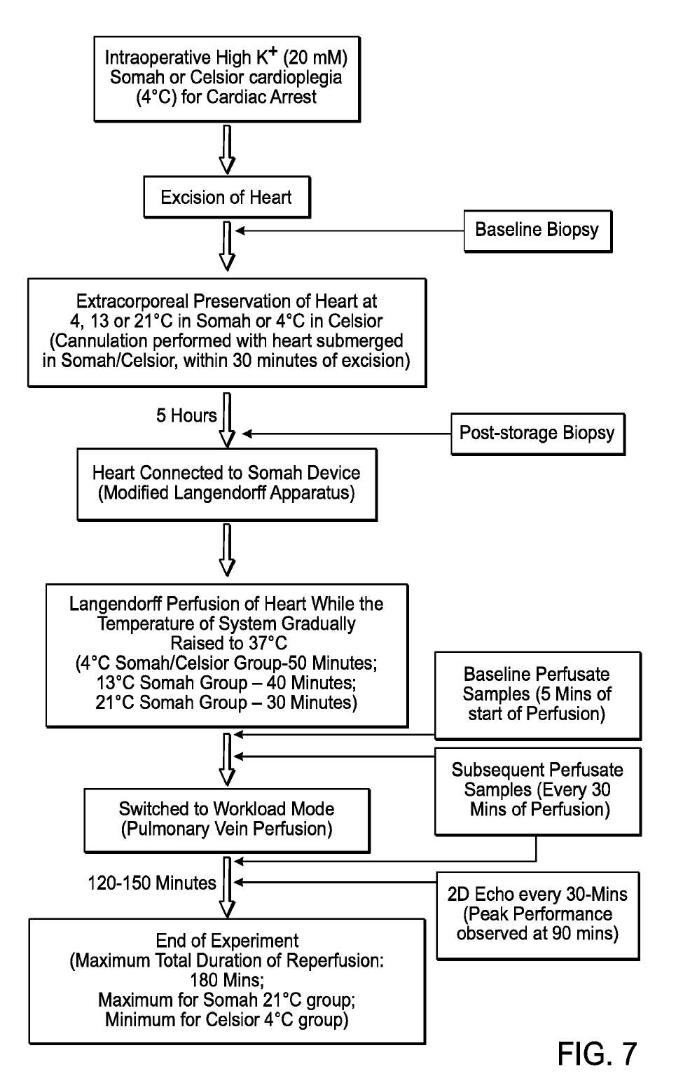

Statistical analyses. All values are expressed as mean±SEM. The analyses were focused on comparing Celsior 4° C. (n=5) with Somah 4° C. heart (n=6), 13° C. (n=6) and 21° C. (n=6) hearts. One-way analysis of variance was performed for all functional measurements (differences in total HEP values in control and at 300 min; MVO₂, lactate, CK, cTnI, coronary flow at 60 min [baseline] and 90 min [peak performance], and ventricular and septal wall thickness) using SigmaPlot (Systat Software Inc., San Jose, Calif.). Tukey test was used to identify specific differences between the groups. Since HEPs are significantly increased in Somah stored hearts, three hearts at random per Somah group and five Celsior hearts were selected for HEP analysis. A paired t-test was used to assess the difference in coronary flow at time 0 and at 37° C., in MVO₂ and lactate at baseline and 90 min within the four groups. For mitochondrial ischemic scores (MIS), four EM slides (×8000 magnification) with 20 mitochondria per slide in Somah hearts (n=3/group) were used. Statistical significance was accepted at the 95% confidence level (p<0.05). Tissue and blood samples for different investigations were obtained as illustrated (FIG. 7).

Results

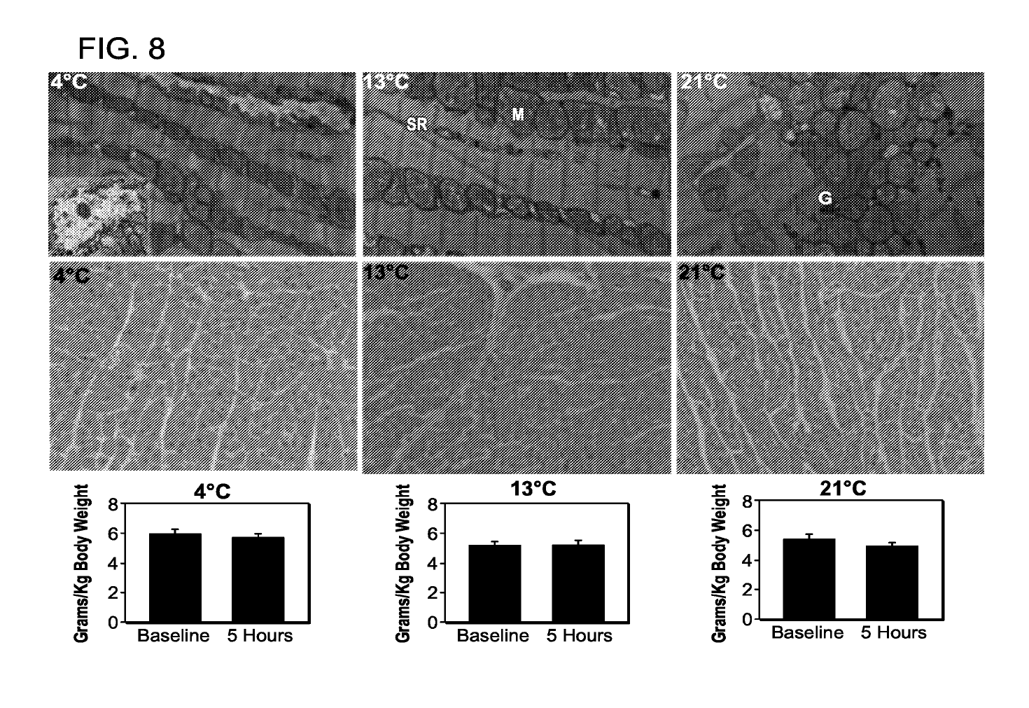

Gross morphology, heart weights, HP and EM. Hearts preserved in Celsior and Somah presented normal morphology and did not show any discoloration. Cardiac dema was estimated in Somah groups by comparing EM, HP and heart weights (FIG. 8). EM and HP demonstrated intact organelle membranes, normal intracellular glycogen content and alignment of contractile proteins, the absence of vacuolation and clear spaces between myofiber bundles and lack of contracture bands, indicating paucity of intracellular edema. Differences in heart weights pre- and post-storage were insignificant. Minimal reversible changes in cardiomyocyte nuclei characterized by accumulation of chromatin beneath nuclear membrane were apparent in Somah groups. Density of mitochondrial matrix was well preserved with insignificant ischemia. Mean MIS, on a scale of 0-6 (6 being worst), demonstrated negligible reversible changes in mitochondria and were 0.09±0.02, 0.17-0.03, 0.07±0.02 in 4° C., 13° C. and 21° C. Somah hearts, respectively. Characteristic irreversible damage such as cristolysis, vacuolation and mitochondrial membrane rupture was not seen in any hearts.

Cardiac Metabolism During Storage

High-energy phosphates: Hearts preserved in Sonmah synthesized HEPs irrespective of storage temperature confirming previous observations. There was a temperature of storage dependent increase in HEP concentration in Somah hearts. Total HEP values of 55.7±5.1, 68.4±11.0 and 81.5±19.8 nM/mg protein in Somah hearts (n=3/group) preserved at 4° C., 13° C. and 21° C. respectively, were significantly greater (p<0.05) than 26.31±1.4 nM/mg protein observed in Celsior hearts (n=5) at end of storage. Control values were not significantly different between the groups.

Lactate production and oxygen consumption during storage: Anaerobic lactate production temporally increased from 0.41 at 2 h to 0.75±0.05 mmol/L in 21° C. hearts but was below detection level in hypothermic Somah groups after 5-h storage. However, the pH remained stable at 7.4±0.1 in all groups. Additionally, hearts stored at higher temperatures vigorously utilized oxygen dissolved in Somah (pO₂ 210-240 mm Hg) due to relatively active aerobic metabolism. The pO₂ decreased by 7.0±7.6, 17.0±3.51 and 14.0±3.51 mm Hg in Somah at 4° C., 13° C. and 21° C., respectively during storage, corresponding with a parallel increase in HEP synthesis. Lactate and pO2 utilization could not be measured in Celsior during storage.

Cardiac Functional Studies

Coronary flow: Upon initiation of antegrade perfusion, coronary flow was observed in all hearts irrespective of solution or temperature of storage. The 4° C. and 13° C. Somah hearts demonstrated slow irregular four chamber contraction as temperature was raised to 21° C., while slow rhythmic contraction were immediately observed after initiation of perfusion in 21° C. group.

In contrast. Celsior hearts demonstrated irregular contractions of atria but not the ventricles. Cardiac contractions and sinus rhythm were enhanced with rising temperature, reaching peak performance at 37° C. approximately 90 min after initiation of perfusion in all Somah groups, but not in the Celsior hearts. Initial coronary flow was significantly greater (p<0.05) in Somah hearts stored at 21° C. than in hearts stored at 4° C. and 13° C. At 378 C, furthermore, there was a significant parallel increase in coronary flow in Somah groups (p<0.05), but not in the Celsior group (Table 2-1). Additionally, coronary flow at 37° C. in Somah groups was significantly greater than in the Celsior hearts (p<0.05).

TABLE 2-1
Alterations in coronary flow (millimeter per minute)
in Celsior 4° C., 13° C., and 21° C.
group hearts upon reperfusion and increasing system temperature.

| Heart group | Time 0 | At 21° C. | At 37° C. |
|---|---|---|---|
| Celsior 4° C. | 323 ± 44 | 223 ± 40 | 350 ± 21¹ |
| Somah 4° C. | 290 ± 21² | 299 ± 25 | 495 ± 38^{1,3} |
| Somah 13° C. | 230 ± 13² | 182 ± 13 | 439 ± 17^{1,3} |
| Somah 21° C. | 312 ± 90² | 312 ± 90 | 621 ± 88^{1,3} |

n = 5 Celsior hearts; n = 6 Somah hearts per temperature group; time 0 = coronary flow immediately upon initial reperfusion.
¹Celsior versus Somah groups at 37° C. (p < 0.05).
²21° C. versus 13° C. and 4° C. group hearts (p < 0.05) at time 0.
³Time 0 versus 37° C. (p < 0.05)

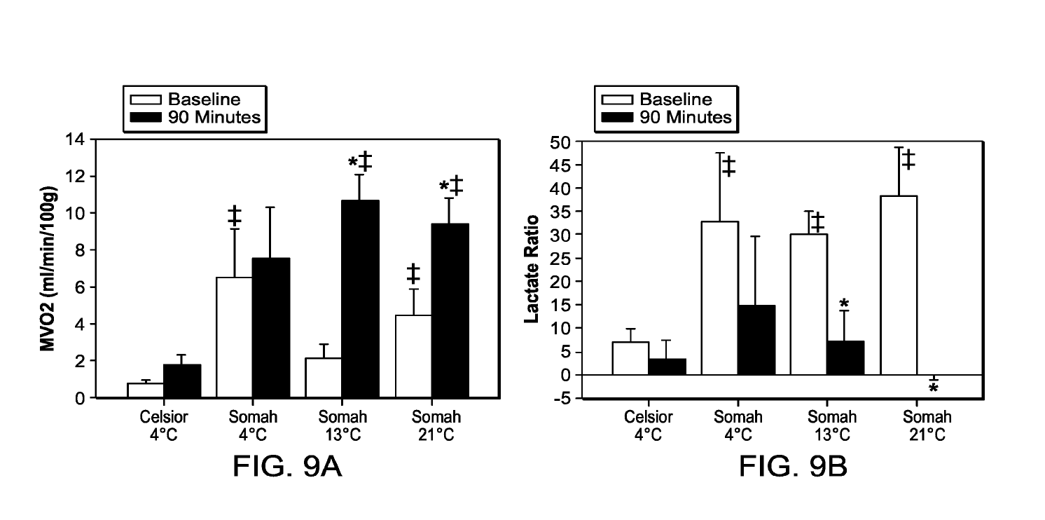

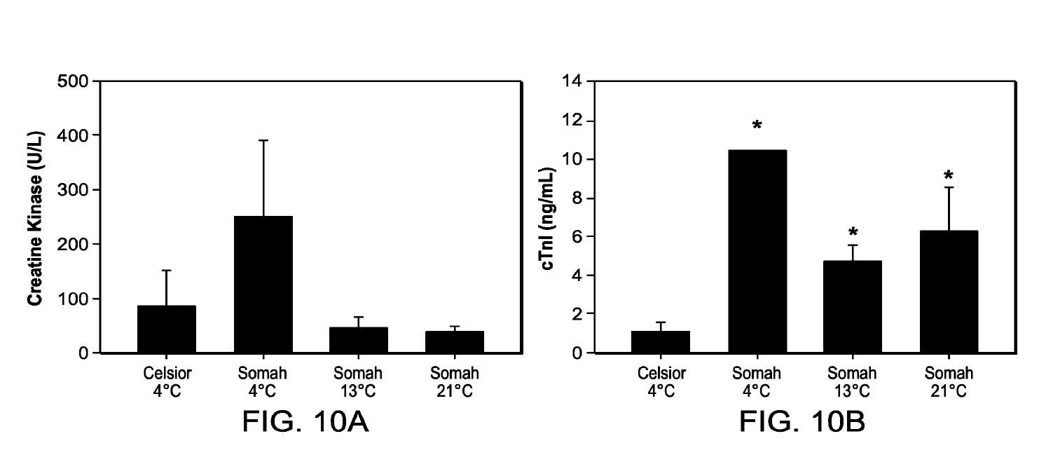

Cardiac metabolism and enzyme release upon reperfusion in working hearts: MVO₂ remained unaltered between 60 (baseline) and 90 min post perfusion in 4° C. Somah hearts and increased significantly within 13° C. and 21° C. Somah hearts, but there was no difference between the three groups. In contrast, MVO₂ was severely attenuated in Celsior hearts (FIG. 9A). Similarly, lactate level in perfusate at baseline was significantly lower in Celsior group (p<0.05) than Somah groups. Lactate decreased in all groups at 90 min, but significantly within the 13° C. and 21° C. Somah groups (p<0.05), though the difference was insignificant between the groups (FIG. 9B). Energy requirements reached steady state at peak performance in all Somah hearts, independent of storage temperature. Ratios of pre- and post-workload HEP levels in cardiac tissue were comparable (˜0.37) in Somah working hearts, but not in Celsior hearts. CK release was lowest in 21° C. Somah group; however, there was no significant difference between any of the groups (FIG. 10A). In contrast, cTnI release was significantly lower in Celsior hearts (p<0.05) compared with the Somah groups (FIG. 10B).

TEE and in vitro 2D Echo imaging: LV anterior wall and septal thicknesses showed no significant change between intraoperative and in vitro reanimation between the four groups (Table 2-2).

TABLE 2-2
Comparison of intraoperative and in vitro thickness
(end-systolic) of the left ventricular (LV) anterior
and septal walls in differentially stored hearts.

| Temperature | | Intraoperative | In vitro |
|---|---|---|---|
| group | LV wall | thickness (cm) | thickness (cm) |
| Celsior | Anterior | 1.29 ± 0.01 | 1.43 ± 0.04 |
| 4° C. | Septal | 1.37 ± 0.13 | 1.54 ± 0.19 |
| Somah | Anterior | 1.37 ± 0.17 | 1.40 ± 0.06 |
| 4° C. | Septal | 1.29 ± 0.12 | 1.36 ± 0.08 |
| Somah | Anterior | 1.73 ± 0.09 | 1.56 ± 0.10 |
| 13° C. | Septal | 1.50 ± 0.09 | 1.44 ± 0.07 |
| Somah | Anterior | 1.56 ± 0.03 | 1.55 ± 0.06 |
| 21° C. | Septal | 1.58 ± 0.08 | 1.71 ± 0.04 |

In vivo and in vitro groups, Celsior, n = 5; Somah; n = 6/group.

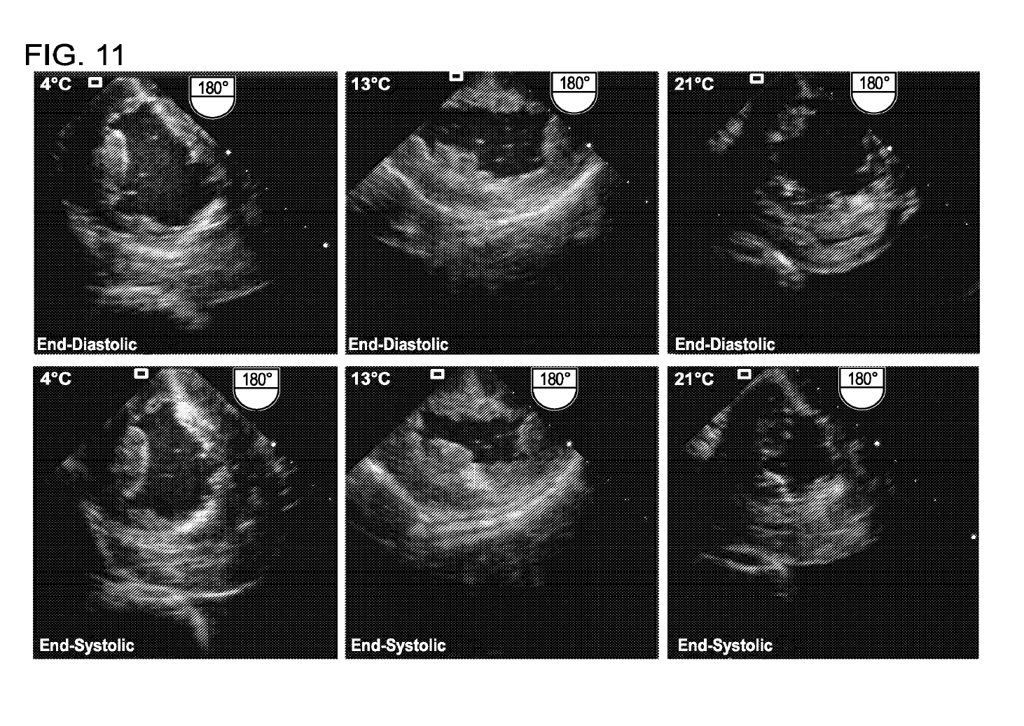

In Somah hearts, 2D Echo visibly demonstrated a temperature-of-storage dependent performance (FIG. 11). In higher temperature groups, a greater degree of wall motion and LV contraction with enhancement of other cardiac functional parameters (FIG. 6) was evident, both during preload (antegrade coronary perfusion) and afterload (PV perfusion). Optimal function of hearts was attained in 21° C. group, at a left atrial pressure of 4±2 mm Hg, in contrast to 12±3 mm Hg for 4° C. and 13° C. hearts, requiring differential stimulatory interventions (Table 2-3).

TABLE 2-3
Numbers of cardioversions and amount of epinephrine
required for functional re-instatement of hearts
stored at 4° C., 13° C. or 21° C.

| | Cardioversion | Epinephrine |
|---|---|---|
| Storage | (33-40 V; epicardial) × | (0.1 mg bolus) × |
| temperature | times delivered | times delivered |
| Celsior 4° C. | >7 | >5 |
| Somah 4° C. | 4-6 | 3-4 |
| Somah 13° C. | 2-5 | 2-3 |
| Somah 21° C. | 1 | — |

In contrast, Celsior hearts were unable to generate synchronous four chamber contractions and were incapable of sustaining any workload, even with multiple stimulatory interventions (Table 2-3), eventually developing stiffness during the experimental period. Therefore, functional data could not be collected from the Celsior hearts. Data obtained at peak performance 90 min into perfusion and full workload in Somah groups was used for comparative analysis. Calculated percentage fractional area change, coronary flow, blood pressure, stroke volume, ejection fraction and cardiac output approached physiological parameters in 21° C. group hearts (Table 2-4).

TABLE 2-4
Cardiac functional parameters during surgery and at peak performance upon extracorporeal reperfusion.

| | | In vitro | | | |
|---|---|---|---|---|---|
| | In vivo | Celsior 4° C. | Somah 4° C. | Somah 13° C. | Somah 21° C. |
| Heart rate (beats/min) | 98 ± 12 | 40 ± 20 | 113 ± 10 | 111 ± 10 | 100 ± 10 |
| LV systolic pressure (mm Hg) | 116 ± 8 | 20 ± 15 | 75 ± 5 | 80 ± 10 | 100 ± 20 |
| LV diastolic pressure (mm Hg) | 68 ± 7 | 5 ± 5 | 15 ± 5 | 35 ± 5 | 40 ± 10 |
| Left atrial pressure (mm Hg) | <10 | nt | 10-15 | 8-10 | 0-5 |
| Cardiac output (mL/min) | 3300 ± 400 | nt | 1250 ± 100 | 2350 ± 350¹ | 2500 ± 300¹ |
| | 2D Echo data analysis | | | | |
| % Fractional area change | >40 | 10% | 21.7 ± 5.54 | 40.08 ± 10.28¹ | 41.58 ± 12.57¹ |
| Ejection fraction (%) | 65 ± 5 | nt | 34.0 ± 6.43 | 56.08 ± 3.72¹ | 57.64 ± 3.54¹ |
| Stroke volume (mL) | 29 ± 4 | nt | 11 ± 3 | 21 ± 5 | 25 ± 2¹ |

2D Echo, 2-dimensional echocardiography; LV, left ventricular; nt, not tested secondary to lack of predicted function.
n = 23, in vivo group; n = 5 Celsior hearts; n = 6 Somah hearts per temperature group.
¹13° C. and 21° C. versus 4° C. group hearts (p < 0.05).

This is the first report demonstrating rapid revival of hearts into a fully functional state within physiologically relevant cardio-hemodynamic parameters with minimal injury after extracorporeal preservation for 5 h at 21° C. This study suggests that, if hypothermic damage can be prevented while enhancing the energy state of an organ during storage, it is possible to maintain structural integrity, cellular homeostasis, repair and recovery of function. Somah solution meets the fundamental requisites of organ storage namely, prevention of edema, Reactive oxygen species-dependent injuries and enhancement of HEP synthesis, thus, mitigating requirements for extreme hypothermic (4° C.) storage and associated cellular injury.

### Example 3

In this example, hearts taken 30 minutes after cardiocirculatory death (DCD hearts) were studied and stored for 4 to 5 times the current clinical norms. Additionally, the study was designed to determine the ideal temperature for the long-term storage of DCD hearts in Somali, in a functionally viable state for transplantation.

Materials and Methods

Animal Model. Three-month-old male Sprague-Dawley Rats were used strictly in accordance with the protocol approved by an Institutional Animal Studies Subcommittee.

Somah Solution Preparation and Other Materials. Somah was formulated as described above. Freshly prepared solution was filter sterilized using 0.4 mm filter (VWR International) stored at 4° C. and used within 24 hours of preparation. All chemicals and antibodies were obtained from Sigma Chemical Co (St Louis, Mo., USA), Amersham Biosciences (Piscatway, N.J., USA), Bio-Rad (Hercules, Calif., USA), or DAKO Corp (Carpinteria, Calif., USA), unless otherwise stated.

Extraction. Storage, and Simulated Reperfusion of the Hearts. Rats rendered unconscious with CO₂ were guillotined and the blood was collected in acid-citrate-dextrose tubes for simulated reperfusion of hearts. DCD hearts were extracted 30 minutes after euthanasia and stored in Somah at 4° C.±2° C., 10° C.±2° C., 21° C.±2° C., or 37° C.±2° C. for 24 hours. At the end of 24-hour storage, simulated reperfusion was performed by incubating hearts in perfusate solution (blood:Somah::3:1) at 37° C. for 30 minutes in a shaking water bath. Heart biopsies were taken before and after reperfusion for live-dead and esterase assays, mitochondrial polarization assays, protein expression, and tissue adenosine triphosphate (ATP) and creatine phosphate (CP) levels.

Live-Dead Viability and Esterase Activity Assays. Viability of cardiomyocytes from Somah DCD hearts was assessed with fluorescence-based live-dead assays and multiphoton microscopy. Heart biopsies, were incubated with calcein AM (acetomethoxy derivative of calcein) and ethidium homodimer dyes (Live-Dead assay kit; Molecular Probes), 10 mmol/L in 1.5 mL HBSS (Hanks Balanced Salt Solution), pH 7.4, for 30 minutes at 21° C., washed with preservation solution and imaged with multiphoton microscope for green (living cells) and/or red (injured/dead cells) fluorescence. Functional integrity of cardiomyocytes was evaluated by semiquantitative measurement of the quantum yield (photon counts) of calcein fluorescence, indicative of esterase activity.

JC-1 Assay of Mitochondrial Membrane Potential. Cardiomyocytes were labeled with JC-1 dye (Molecular Probes), imaged, and the mitochondrial polarity ratios were determined using multiphoton microscopy.

Protein Extraction and Western Blotting. LV tissue (20 mg) was cut into 300 pieces, suspended in 200 mL of Lysis buffer (CellLytic MT; Sigma-Aldrich) with a protease inhibitor cocktail, homogenized for 30 seconds before centrifuging at 16.000 g for 10 minutes and the supernatant (total protein) collected and then quantitated using Bio-Rad protein assay kit. Proteins were resolved on 7.5%, 10% or 12% SDS-PAGE and transferred to nitrocellulose membrane. Blots were incubated with primary antibodies (1:1000; anti-myosin heavy and light chain, actinin, actin, and troponin C) followed by HRP (horseradish peroxidase) conjugated secondary antibodies and protein expression detected using electrochemiluminescence (GE Hlealthcare).

ATP and CP Assays. ATP and CP levels were measured in cardiac tissue after 24-hour storage at 4° C., 10° C., 21° C. or 37° C. and upon reperfusion using spectrofluorometer and bioluminescent assay kit (Perkin Elmer, Waltham, Mass. USA).

Multiphoton Imaging. State-of-the-art multiphoton imaging techniques using BioRad imaging system (MRC 1024ES) coupled with MaiTai mode-locked titanium:sapphire laser (Spectra-Physics, Mountain View, Calif., USA), were used for deep-imaging cardiac biopsies and semiquantitative fluorescence measurements. Images 512×512 pixels were collected in direct detection configuration, at pixel resolution of 0.484 mm. Cardiomyocytes were identified by XYZ axis scanning, and imaged at depths of 50 mm in LV biopsies from the point of excision.

Statistical Analysis. Metamorph software (Molecular Devices, Downingtown, Pa., USA) was used for data extraction, quantification, and analysis of fluorescence counts from multiphoton images, in blinded fashion by different members of the lab. Data are expressed as means±standard errors. Differences between groups were determined using Student t test (paired on unpaired) or 1-way analysis of variance where applicable. Statistical significance was accepted at the 95% confidence level (P<0.05). Data was derived from n=3 for each temperature group, n=25 measurements for esterase activity and mitochondrial polarization, and n=5 for ATP and CP level determination.

Results

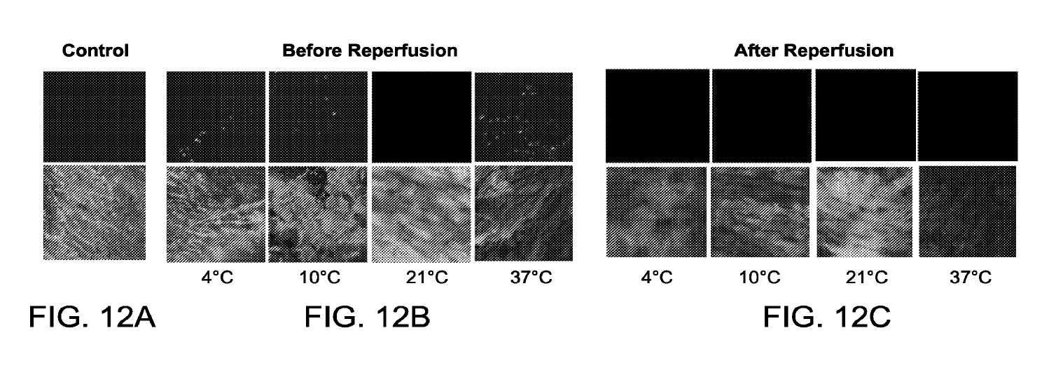

Cardiomyocyte viability in extracted hearts. Live-dead assays and multiphoton microscopy (FIG. 12) of cardiac biopsies performed immediately after extraction (control; FIG. 12A) demonstrated robust green fluorescence (indicative of viable cells) but no red nuclear fluorescence (indicative of damaged cells). After 24-hour storage in Somah, with the exception of discoloration and visible loss of structural integrity in the 37° C. group, the gross morphology of DCD hearts was well preserved at all other temperature groups while the green fluorescence of live-dead assay in these groups was similar to that of controls. However, at 4° C., 10° C., and 37° C., the red nuclear fluorescence of damaged cells was also enhanced after 24-hour storage (FIG. 12B) suggesting that hearts stored at all, but the subnormothermic temperature (21° C.) suffered some concurrent cellular damage. Upon simulated reperfusion of the stored hearts, there was a demonstrable decrease in green fluorescence in hearts that were stored at 4° C. and 37° C. (FIG. 12C).

Esterase Activity Measurement. Prior to simulated perfusion, quantum yield of green fluorescence as a function of esterase activity was highest in the hearts stored at 21° C. and lowest at 37° C. (Table 3-1). After reperfusion, the esterase activity was unaltered in hearts stored at 21° C. but was variably decreased in hearts stored at 4° C., 10° C., and 37° C., although not significant in 10° C. group.

TABLE 3-1
Quantitative assessment of calcein fluorescence
(esterase activity) in DCD hearts.

| | 4° C. | 10° C. | 21° C. | 37° C. |
|---|---|---|---|---|
| Before | 193.4 ± 6.7 | 202.8 ± 7.9 | 216.8 ± 8.4 | 130.5 ± 5.9 |
| reperfusion | | | | |
| After | 165.8 ± 6.9 | 191.8 ± 6.7 | 220.4 ± 6.6 | 113.6 ± 5.8 |
| reperfusion | | | | |
| % change | −14%^{b} | −5% | 2% | −13%^{b} |
| in CF | | | | |

Quantitation data (total fluorescence count) was obtained from analysis of images depicting esterase activity from 3 hearts donated after cardiocirculating death in each temperature group after 24 hours of storage in Somah solution and upon reperfusion. After 24 hours of storage (before reperfusion), the esterase activity was lowest in 37° C. and highest in 21° C. group hearts. Upon reperfusion, a significant decline in esterase activity was evident in hearts preserved at extreme hypothermic (4° C.) or normothermic (37° C.) temperatures but was unaltered in the 10° C. and 21° C. group hearts. CF = calcein fluorescence.
^{a} Arbitrary units ± standard error of means
^{b}Significant change after perfusion

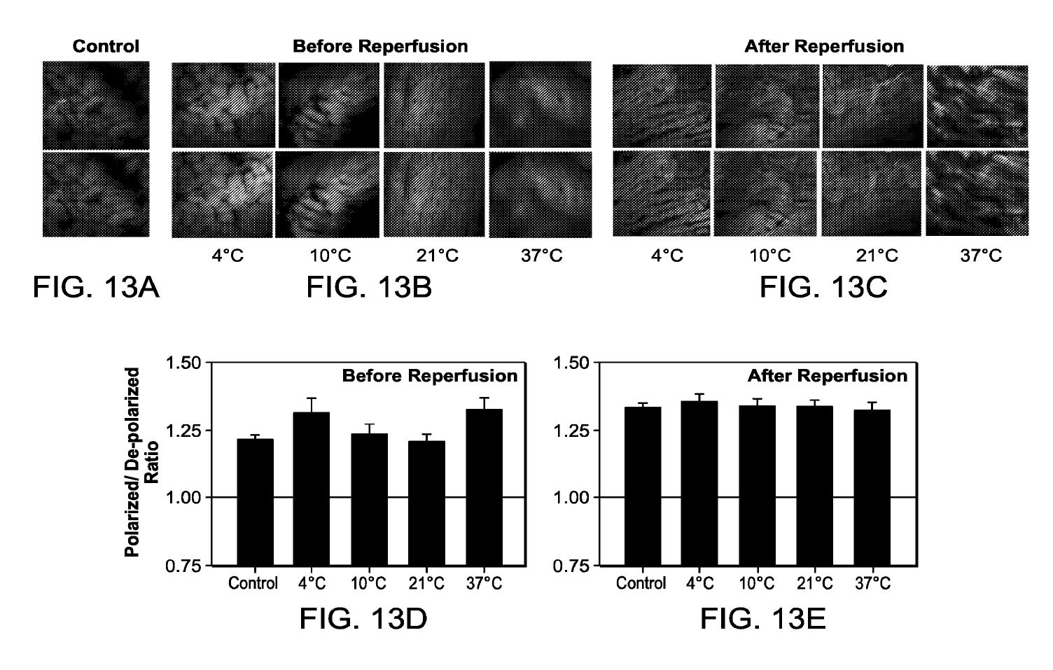

JC-1 Assay for Mitochondrial Polarization. In the freshly extracted DCD hearts, the polarized and depolarized states of mitochondria were in equilibrium (FIG. 13). Irrespective of storage temperature, mitochondrial membranes in cardiomyocytes of DCD hearts remained polarized both after 24-hour storage and upon reperfusion, while the polarization ratio was not altered in any group after reperfusion. Furthermore, there was no significant difference in mitochondrial polarization status in the different groups before or after reperfusion.

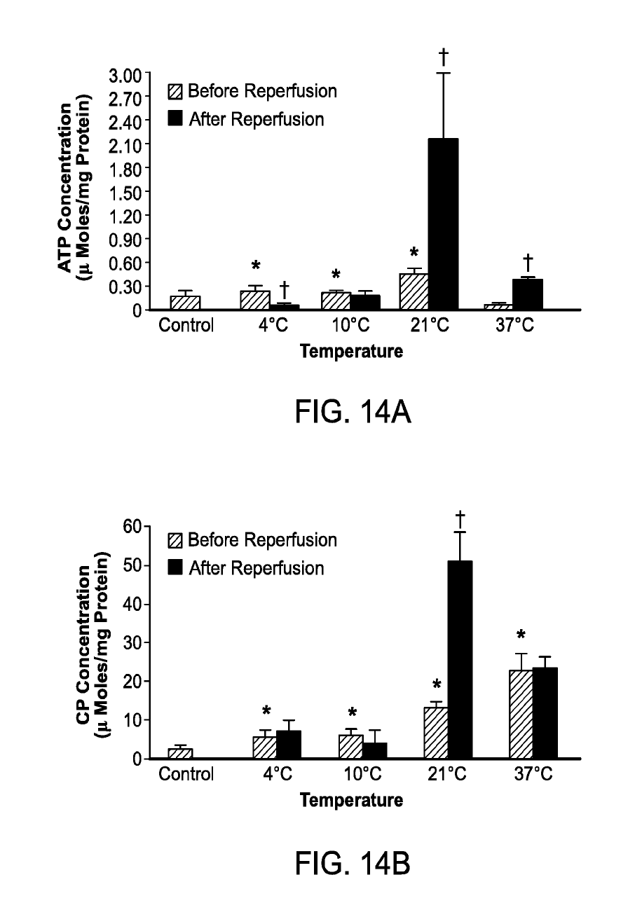

High-Energy Phosphate Syntheses. Compared to controls, ATP/CP syntheses in hearts were significantly enhanced after 24-hour storage in Somah at 4° C., 10° C., and 21° C. (FIG. 14). Upon reperfusion, ATP synthesis was significantly decreased in 4° C. group but was unchanged in the 10° C. group. In contrast, reperfusion resulted in a 400% increase in ATP synthesis in hearts stored at subnomiothermic temperature. Parallel to the ATP studies, CP synthesis was also significantly increased upon reperfusion in 21° C. group.

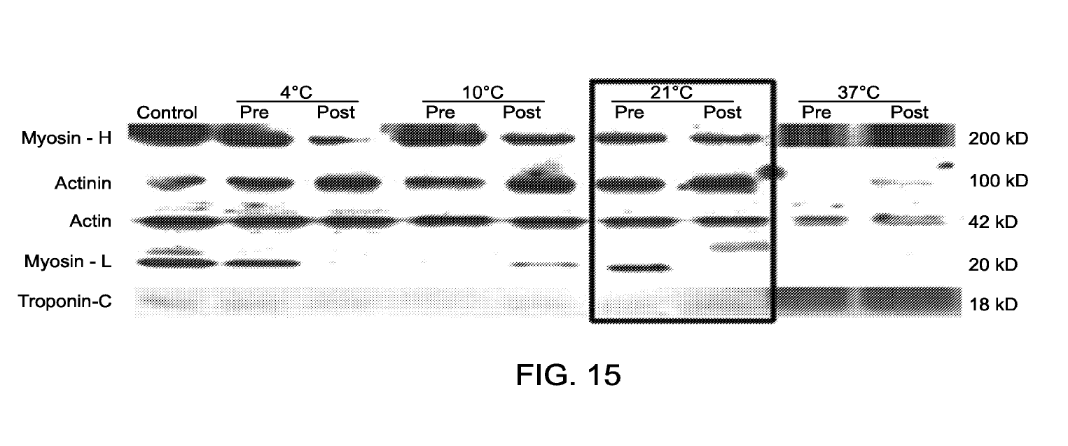

Protein Expression The structural viability of ex vivo stored hearts was determined by assessing the expression of proteins important for contractile function. Expression of myosin HC, actinin, actin, myosin LC and troponin C was well preserved in hearts stored in Somah for 24 hours at 21° C. (FIG. 15). In contrast, these proteins were variably lost in other temperature groups either after 24-hour storage or upon reperfusion.

In conclusion, this example demonstrated for the first time that DCD hearts in static storage can be preserved in a viable state at subnormothermic temperature, beyond currently acceptable time of 4 to 5 hours of hypothermic storage, using the recently designed organ preservation solution, Somah.

### Example 4

This Example evaluates the relative efficacy of Somah at 4 and 21° C. in preservation of optimum heart function after in vitro storage at subnormothermia.

Materials and Methods

Heart procurement surgery and cardioplegia. Ten female Yorkshire swine (45-54 Kg) were used in this comparative study. Hearts were divided into two groups of either 4° C. (n=5) or 21° C. cardioplegia (n=5), as per the protocol approved by an Animal Studies Committee (Institutional Animal Care and Use Committee). Hearts were extracted using mediastinal approach as described (Thatte H S, Rousou L, Hussaini B E, Lu X G, Treanor P R, Khuri S F: Development and evaluation of a novel solution, Somah, for the procurement and preservation of beating and non-beating donor hearts for transplantation. Circulation. 2009, 120: 1704-1713). The animals were bled from femoral vessels to collect blood for ex vivo experiments, and aorta was clamped when systolic pressure fell below 40 mmHg. 1000 ml of SOMAH cardioplegia (SOMAH (Thatte H S. Rousou L, Hussaini B E. Lu X G, Treanor P R, Khuri S F: Development and evaluation of a novel solution, Somah, for the procurement and preservation of beating and non-beating donor hearts for transplantation. Circulation. 2009, 120: 1704-1713) modified by addition of 20 mM K+, final concentration), at 4 or 21° C. was infused into the aortic root at a pressure of 75-100 mmHg at a flow rate of 300-400 ml/minute using roller pump and pressure transducer (Myothermnn Cardioplegia System, Medtronics, Minneapolis, Minn., USA) and the data was recorded using iWorks system (Dover, Nil, USA). After cardioplegic arrest, heart was dissected from all attachments and rinsed with normal saline before storing in SOMAH for 5-hours at 21° C. Hearts were transported to the lab within 15 minutes of excision.

Extracorporeal storage of heart. Hearts were placed in sterile zip-lock bags containing 2 L of SOMAH in water-jacketed water bath at 21±2° C. The temperature of preservation solution was checked regularly during the entire storage period. Hearts were maintained in a non-contractile state by increasing SOMAH's plegia potential by supplementing the solution with 20 mM K+ complemented by 37 mM Mg2+(Fukuhiro Y, Wowk M, Ou R, Rosenfeldt F, Pepe S: Cardioplegic strategies for calcium control: low Ca2+, high Mg2+, citrate, or Na+/H+exchange inhibitor HOE-642. Circulation. 2000, 102 (19-3); Osaki S, Ishino K, Kotani Y, Honjo O, Suezawa T. Kanki K, Sano S: Resuscitation of non-beating donor hearts using continuous myocardial perfusion: the importance of controlled initial reperfusion. Ann Thorac Surg. 2006, 81: 2167-2171), during storage. Hearts in each group were weighed prior to and after 5-hour storage upon carefully emptying the heart chambers. Tissue punch biopsies (2×4 mm) were taken in the lab from the posterior wall of LV, 15 minutes into (0 hour; control) and after 5 hour storage for HEP assays.

ATP and creatine phosphate assay. ATP and creatine phosphate (CP) were measured in tissue extracts as described (Thatte H S. Rousou L, Hussaini B E, Lu X G, Treanor P R, Khuri S F: Development and evaluation of a novel solution, Somah, for the procurement and preservation of beating and non-beating donor hearts for transplantation. Circulation. 2009, 120: 1704-1713; Bessho M. Ohsuzu F, Yanagida S, Sakata N, Aosaki N, Tajima T. Nakamura H: Differential extractability of creatine phosphate and ATP from cardiac muscle with ethanol and perchloric acid solution. Anal Biochem. 1991, 192: 117-124). In brief, tissue biopsies were flash frozen and stored at −80° C.; 20 mg of tissue was suspended in 400 μl of 0.4 M ice-cold perchloric acid and homogenized twice for 30 seconds. Homogenate was centrifuged at 1970 g for 10 minutes at 0° C. An aliquot of supernatant was neutralized with equal volume of ice-cold 0.4 M KHCO₃ and centrifuged as above. The supernatant was stored at −80° C. for ATP and CP measurements. The pellet was dissolved in equal volume of 0.1 M NaOH, centrifuged and used for protein assay. ATP and CP were measured using a bioluminescent assay kit (Sigma-Aldrich and GloMax-Multi+Detection System, Promega), according to the protocol provided by the manufacturer.

Preparation of heart for ex vivo resuscitation and functional studies. Aorta and pulmonary artery (PA) were separated. Aorta was cannulated (½-⅜inch tubing connector) and coronaries were gently flushed with 100 ml of SOMAH in both 4° C. and 21° C. cardioplegia groups at 40-50 mmHg pressure, carefully avoiding entry of air into the aorta. Pulmonary veins (PV) were separated and cannulated with ½-¼ inch tubing connector. PA was cannulated for sample collection while superior and inferior vena cavas were ligated.

Preparation of blood for ex vivo studies. Systemically heparinized blood was collected intraoperatively, leukodepleted (Pall Leukoguard filter) and stored at 4° C. Prior to experiments, perfusate was prepared by adjusting the hematocrit of blood to 20% using SOMAH solution (1:1 ratio to reduce viscous strain on heart) and warmed to 21° C. The perfusate, pH, glucose, K+, Ca2+ and HCO₃— were adjusted for swine blood levels (7.5; 100 mg/dl; 3.7, 1.38, and 32 mmol/l respectively), using 10% dextrose, KCl, CaCl₂ and NaHCO₃, respectively, as required.

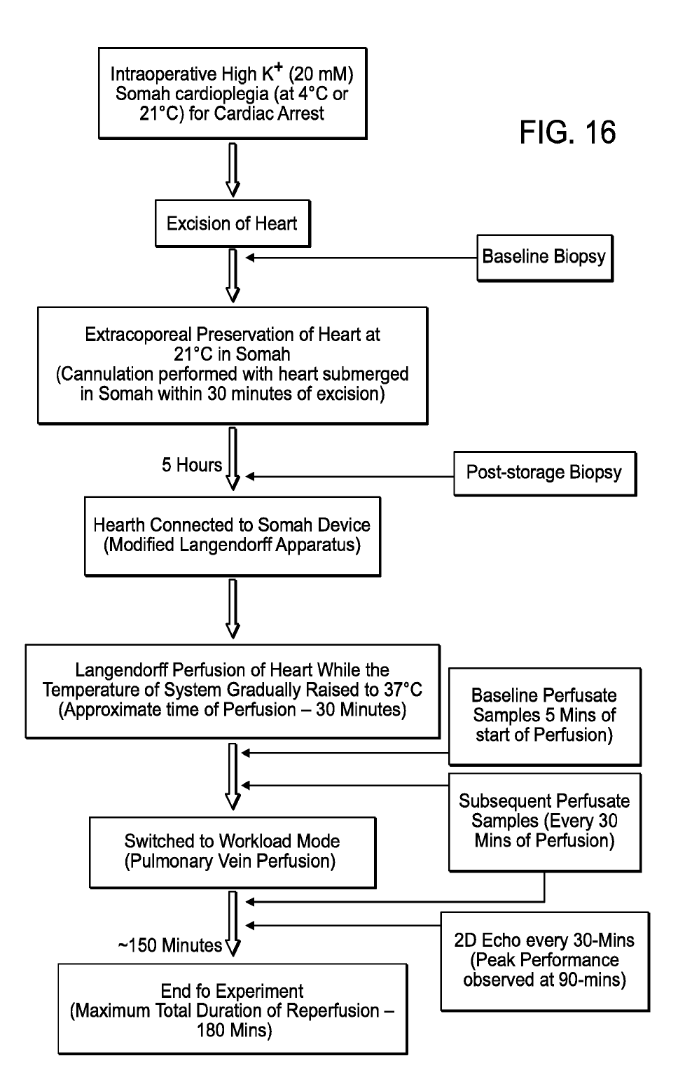

The SOMAH device. A custom-built apparatus was used for extra-corporeal reanimation of hearts (FIG. 1). CDI monitor (Clinical Documentation Improvement monitoring system 500, Terumo cardiovascular systems corporation. Ann Arbor, Mich.), was used for real-time monitoring of perfusate pH, temperature, pO₂, pCO₂, K+ and HCO₃—. These parameters were also analyzed in inflow/outflow samples using i-STAT analyzer (Abaxis Ltd, Union city, CA). Pressures and flows were recorded at various points in the circuits (FIG. 16). A trans-esophageal echocardiography (TEE) probe was used to assess the contractile function of heart using the 2D-Echo system. 3-lead ECG was recorded during 2D Echo using brass crocodile leads immersed in the perfusate surrounding the heart. Pressures and flow data was acquired and monitored in real time using HMI software, specifically written for SOMAH Device (Comdel Inc, Wahpeton, N. Dak.).

Ex vivo functional studies. Hearts were attached to SOMAH Device and perfused through the aorta at 40-60 mmHg for 5 minutes, with 1-1.5 liters of SOMAH at 21° C. (pH 7.5), and then with perfusate, till pH, blood gases and electrolyte equilibrium was established. The perfusate pH, glucose, K+, Ca2+, HCO₃— were adjusted for swine blood levels as mentioned above. Strong cardiac contractions were noted in both groups as the system temperature was gradually raised to 37° C. over a 30 minute period. Hemodynamic steady state (with respect to pH, blood gases, and electrolytes) was achieved within 40 minutes. Total duration of the experimental perfusion was approximately 180 minutes. Hearts were perfused through aortic root (no workload) until the system temperature reached 37° C., after which PV perfusion (full workload) proceeded until end of experiment. Corollary blood flow was determined during the initial antegrade perfusion by the amount of perfusate flowing to the heart through aorta per minute, and in the working heart by the amount of perfusate collected from pulmonary artery (both cavas ligated) per minute. Electroconversion (40-50 J) and/or epinephrine (1:50,000-1:100,000) were used if required (Lowalekar S K, Cao H, Lu X G, Treanor R, Thatte H S: Subnormothermic preservation in SOMAH: a novel approach for enhanced functional resuscitation of donor hearts for transplant. Am J Transplant. 2014). Epicardial 2D Echo was performed using TEE probe for functional assessment at 60 minutes (baseline) and at peak performance, approximately 90 minutes after initiation of perfusate perfusion in the two groups with hearts under full workload; and every 30 minutes thereafter. Peak cardiac performance was defined by the maximum contractile activity observed by 2D Echo. The data at peak performance was used for comparisons between the two groups.

Enzyme assays and blood chemistry. Quantitative levels of cardiac creatine kinase (CK), aspartate aminotransferase (AST), troponin-I (cTnI), lactate and gases (pO₂/pCO₂) were measured intra-operatively and in SOMAH samples taken at 10 minute, 2-hour and at end of 5-hour heart storage using Vetscan VS2 or iStat (Abaxis Ltd, Union City, Calif.). Inflow (aortic) and outflow (PA) samples were collected for enzyme assays and post perfusion assessment of myocardial O₂ consumption (MVO₂) and lactate levels using Vetscan VS2 or i-Stat System, at 5 and 90 minutes for enzyme assays, and at 60 minutes (baseline) and 90 minutes (peak performance) for MVO2 and lactate, after start of perfusate perfusion with Vetscan or iStat. MVO2 was calculated as described (Klabunde R: Cardiac function. Cardiovascular Physiology Concepts. 2011, Lippincott Williams & Wilkins, Baltimore, Md. USA, 84-88).

Epicardial echocardiography. A trans-esophageal (TEE) probe was used for 2D echo evaluation of cardiac function intra-operatively and ex vivo using the Acuson Cypress system (Acuson, Mountain View, Calif.) and images were analyzed using Cypress viewer software provided with the system. Hearts were connected to SOMAH Device and suspended in a chamber containing 2 L of perfusate that covered ⅔rd surface of the heart. ECG was recorded during entire course of the experiment and 2D Echo acquisition was begun approximately 45-60 minute after perfusion, when good cardiac contractions were observed, and repeated at 30-minute intervals. Probe was placed in direct contact with heart and angle of probe and direction of pulse were adjusted as to obtain short-axis and long-axis views for calculations of cardiac functional parameters, and ventricular wall and septal thickness.

Statistical analyses. Equal number of animals (n=5), were assigned to 4° C. and 21° C. cardioplegia groups for comparative analysis for biochemical, hemodynamic and functional measurements from each group. Statistical comparison for significant differences between the two groups was performed using SigmaPlot software. Paired t-test was used for all comparisons. P-value of <0.05 was considered significant. All values are expressed as mean±SEM. Flow diagram of the experimental design is shown in FIG. 16.

Results

Intraoperative cardioplegia. Cardiac arrest was dependent on the temperature of cardioplegia and occurred within 10-15 seconds in 4° C. group and 20-25 seconds in the 21° C. group, likely because the hypothermic (4° C.) component of cardiac arrest was deliberately eliminated in the latter.

Gross morphology, heart weights and release of enzymes during storage. Irrespective of temperature of cardioplegia, all hearts presented normal gross morphology without any discoloration. Hearts were pliable with no signs of hardening or stiffness. Weights of the hearts during 5-hour storage in the two groups were not altered between pre and post storage, demonstrating lack of storage-induced gross edema (not shown). A time dependent release of cardiac enzymes was minimally apparent in both groups that were not significantly different (not shown).

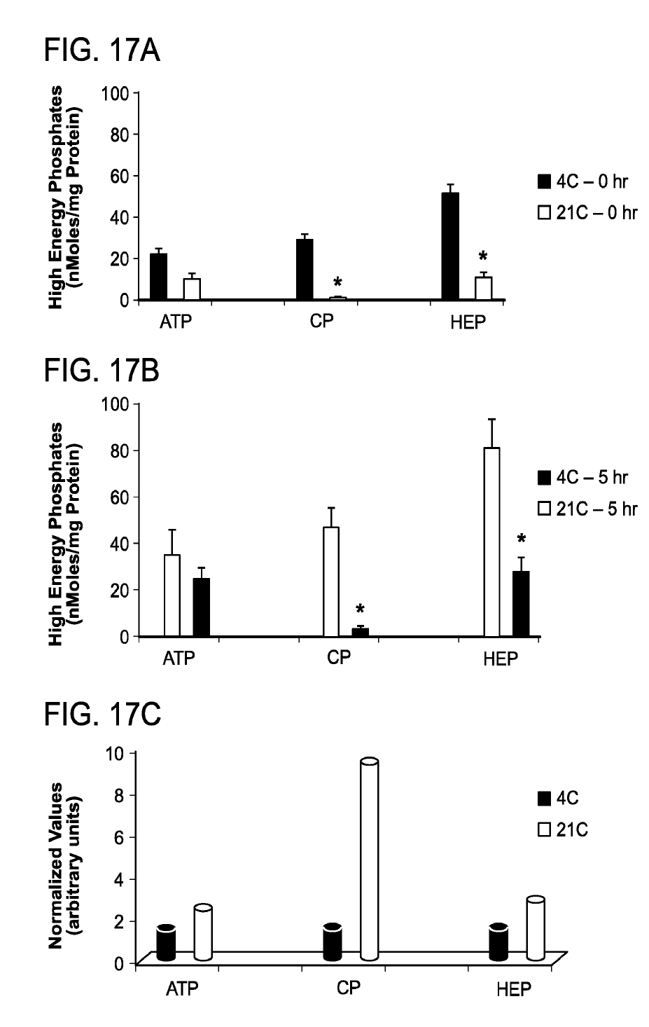

Cardiac tissue HEP levels after arrest and during storage. As shown in FIG. 17, concentration of ATP. CP and total high-energy phosphates (HEP), within 15 minutes (control) of cardiac arrest were significantly greater in 4° C. as compared to 21° C. cardioplegia hearts (P<0.001) possibly due to the greater consumption of energy in 21° C. group hearts as they required approximately 10 seconds longer for total arrest. Both groups actively synthesized CP and ATP during storage, as shown in FIG. 17B. While the total concentrations of HEP at the end of storage were significantly greater in 4° C. hearts (P<0.01), normalization of values at 5 hour with respect to those at 0 hour demonstrated greater availability of HEP in 21° C. hearts than in 4° C. hearts at the end of storage, as shown in FIG. 17C.

Ex vivo cardiac functional studies—Coronary flow upon reperfusion. Coronary flow through the aorta, upon initial antegrade perfusion at similar perfusion pressures, was significantly greater in 21° C. hearts (P<0.05) than the 4° C. group (Table 4-1). The hearts in both groups demonstrated slow four chamber contractions immediately upon initiation of perfusion. Coronary flow decreased initially when system temperature was raised to 30° C., as the hearts started contracting vigorously. Both pressure and flow increased as system temperature was raised to 37° C. Coronary flow was highest in both groups at 37° C. (Table 4-1).

TABLE 4-1
Coronary flow (ml/min) in differential temperature cardioplegia hearts with the rise in system
temperature of SOMAH Device

| Heart groups | P1-21° C. | F1-21° C. | P2-30° C. | F2-30° C. | P3-37° C. | F3-37° C. |
|---|---|---|---|---|---|---|
| 4° C. | 40 ± 1 | 312 ± 90 | 44 ± 2 | 297 ± 28 | 53 ± 4 | 621 ± 88 |
| 21° C. | 40 ± 1 | 464 ± 65* | 40 ± 2 | 348 ± 64 | 50 ± 3 | 619 ± 34 |

P1, P2, P3 - Aortic Root Pressures at respective temperatures.
F1, F2, F3 - Coronary Flows at respective temperatures.
*Significant from 4° C.

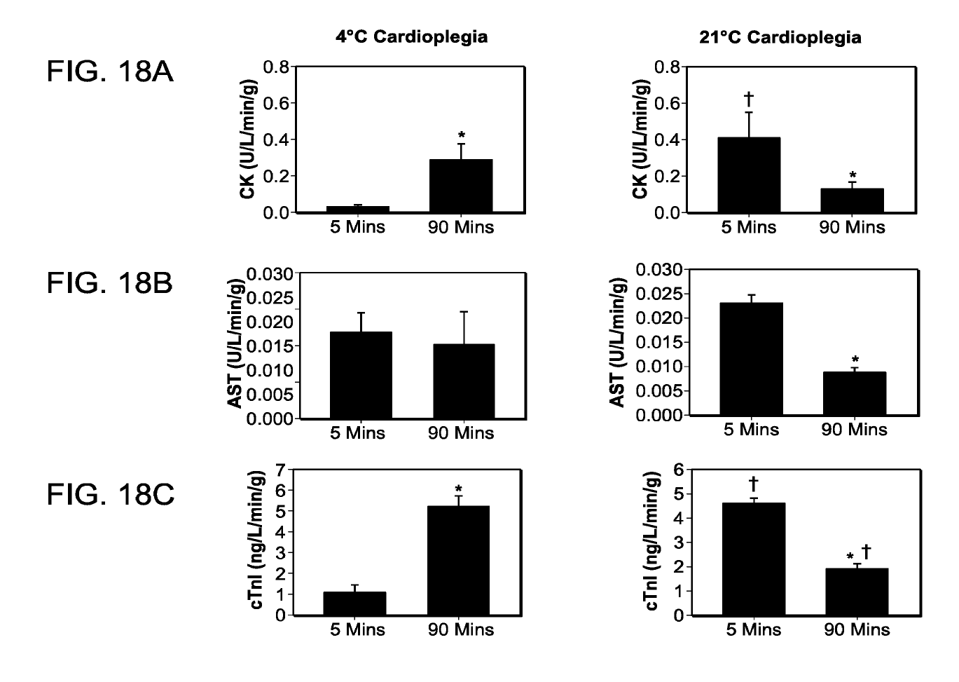

Release of enzymes upon reperfusion. Rate of CK. AST and cTnI release increased in 4° C. hearts during the perfusion period (FIG. 18). Both CK and cTnI release increased significantly with the time of perfusion, but the AST did not. In contrast, there was a temporal decrease in release of these three enzymes in the 21° C. hearts during the same period (FIG. 18). However, release of CK and cTnI, but not the AST, was significantly greater by the 21° C. hearts upon initiation of perfusion than by the 4° C. hearts.

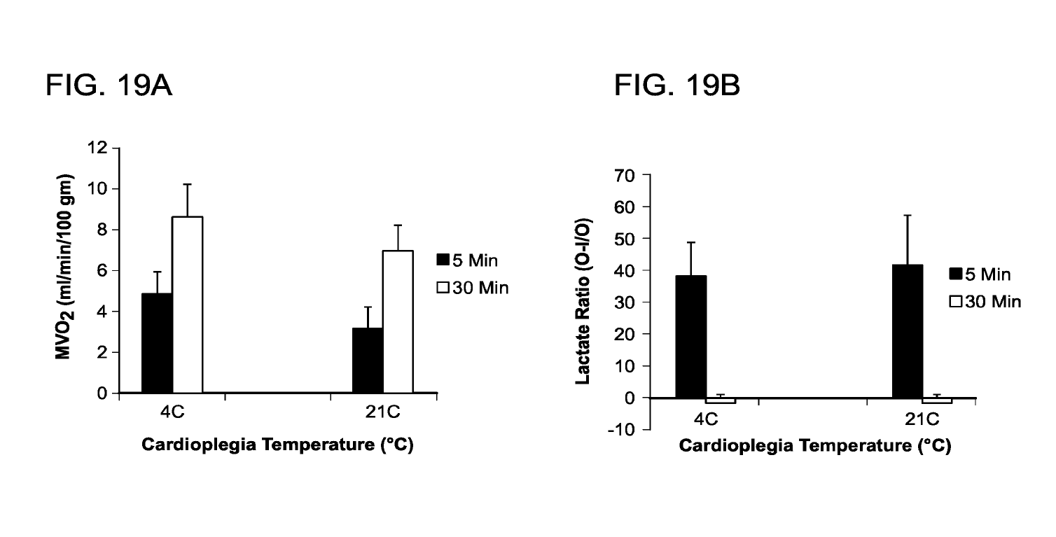

Metabolism in reperfused hearts. Consistent with previous observations (Lowalekar S K, Cao H, Lu X G, Treanor R, Thatte H S: Subnormothermic preservation in SOMAH: a novel approach for enhanced functional resuscitation of donor hearts for transplant. Am J Transplant. 2014), there was a rapid switch in metabolism from anaerobic to aerobic upon reperfusion in both 4° C. and 21° C. cardioplegia groups, demonstrated by an increase in oxygen consumption and reversal of lactate ratios at peak performance, 90 minutes into reperfusion (FIGS. 19A and 19B). Oxygen extraction, lactate production and utilization, reached a steady state in both groups at peak performance that were not significantly different. However, upon reperfusion, 21° C. group hearts demonstrated robust synthesis of HEP in the working hearts. In contrast, production was attenuated in 4° C. hearts and the HEP continued to decline during course of the experiment. Ratios (post perfusion/pre perfusion) of ATP. CP and total HEP were 1.10, 1.97 and 1.17, respectively in 21° C. hearts which were significantly greater (p<0.01) than ratios of 0.47, 0.32 and 0.38 observed in the 4° C. hearts at end of experiment, when post perfusion biopsies were taken for HEP assays.

Functional revival upon reperfusion. Immediate spontaneous activity of both the atria and ventricles was apparent upon commencement of reperfusion in both groups. With increased temperature, force of ventricular contraction peaked at about 37° C. after a single cardioversion, which also established the normal electrical activity and electromechanical coupling in sinus rhythm in both groups. While 4 of the 5 hearts in 21° C. cardioplegia group reverted to sinus rhythm with a single cardioversion and none requiring further inotropic support, 2 of the 5 hearts in 4° C. cardioplegia group required additionally a single dose of epinephrine to maintain optimal function. Interestingly, hearts in both groups remained pliable throughout the experiment and did not show any edema at peak performance as indicated by unaltered thickness of LV and the septum, Table 4-2. The comparative data between 4 and 21° C. cardioplegia groups for cardiac functional parameters acquired by 2D Echo were similar to those observed in vivo (Table 4-2). Although hearts that received SOMAH cardioplegia at 21° C. appeared to have a better recovery, there were no significant differences in the functional parameters of the two groups.

TABLE 4-2
Cardiac functional parameters during surgery and at
peak performance upon extracorporeal reperfusion in
4° C. and 21° C. SOMAH cardioplegia group hearts

| Parameters | In vivo | 4° C. hearts | 21° C. hearts |
|---|---|---|---|
| LV ant wall thickness (cm) | 1.48 ± 0.07 | 1.55 ± 0.06 | 1.51 ± 0.04 |
| Septal thickness (cm) | 1.43 ± 0.12 | 1.61 ± 0.04 | 1.49 ± 0.07 |
| Heart rate | 98 ± 12 | 100 ± 10 | 110 ± 10 |
| LV systolic pressure (mm Hg) | 116 ± 8 | 100 ± 20 | 110 ± 10 |
| LV diastolic pressure | 68 ± 7 | 40 ± 10 | 55 ± 10 |
| (mm Hg) | | | |
| Left atrial pressure (mm Hg) | <10 | 0-5 | 0-5 |
| Cardiac output | 3300 ± 400 | 2500 ± 300 | 2640 ± 250 |
| Fractional area change (%) | >40 | 42 ± 13 | 51 ± 4 |
| Ejection fraction (%) | 65 ± 5 | 58 ± 4 | 66 ± 5 |
| Stroke volume (ml) | 29 ± 4 | 25 ± 2 | 24 ± 3 |

This Example was undertaken to evaluate whether using crystalloid SOMAH at 21° C. instead of standard temperature of 4° (for cardioplegia could further improve the quality of hearts preserved at ambient temperature in SOMAH, and their eventual reanimation in vitro into optimal function.

Myocardial edema has been reported in hearts arrested at perfusion pressures of cardioplegia as low as 50 mmHg (Mehlhorn U, Geissler H J, Laine G A, Allen S J: Myocardial fluid balance. Eur J Cardiothorac Surg. 2001, 20: 1220-1230). However, in present and past studies a crystalloid SOMAH cardioplegia infusion pressure of 100 mmHg has consistently been used irrespective of cardioplegia temperature, but edema was not seen in any of the hearts (Lowalekar S K, Cao H, Lu X G, Treanor P R, Thatte H S: Sub-normothermic preservation of donor hearts for transplantation using a novel solution, SOMAH: a comparative pre-clinical study. J Heart Lung Transplant. 2014, 33 (9): 963-970). SOMAH cardioplegia provides all the advantages of blood cardioplegia, in terms of protection from cardiac edema and provision of substrates for energy metabolism, and also provides clear surgical field. Additionally, provision of physiological concentrations of calcium in SOMAH solution, also prevents the likelihood of myocardial damage due to ‘calcium paradox’ (Yamamoto H, Yamamoto F: Myocardial protection in cardiac surgery: a historical review from the beginning to the current topics. Gen Thorac Cardiovasc Surg. 2013, 61: 485-496. 10.1007/s 1748-013-0279-4). Furthermore, the disadvantages of blood such as the presence of leukocytes and platelets, culpable in reperfusion injury (Han S, Huang W. Liu Y, Pan S, Feng Z, Li S: Does leukocyte-depleted blood cardioplegia reduce myocardial reperfusion injury in cardiac surgery? A systematic review and meta-analysis. Perfusion. 2013, 28 (6): 474-483), are also averted. In contrast, the other crystalloid solutions (Celsior and UWS), when used for cardioplegia in recent studies did not prevent loss of high-energy phosphates in the stored hearts, edema upon reperfusion and potential high K+ mediated calcium overload and stiffness that resulted in non-functioning hearts (Lowalekar S K, Cao H, Lu X G, Treanor R, Thatte H S: Subnormothermic preservation in SOMAH: a novel approach for enhanced functional resuscitation of donor hearts for transplant. Am J Transplant. 2014).

In this study, HEP were conserved in 4° C. hearts because of rapid arrest, as a result total concentration of HEP was also greater in these hearts at the end of storage. In contrast, despite K+ concentration (20 mM) being equal in the two groups, the 21° C. hearts took longer for total arrest because of absence of the hypothermic component, resulting in depletion of HEP. Both groups synthesized HEP during storage in SOMAH, however, the functional availability of HEP was greater in 21° C. hearts than the 4° C. hearts at the end of 5 hour (FIG. 3). Similarly, upon reperfusion, 21° C. hearts continued to synthesize HEP to meet the demands of the working heart, unlike the 4° C. hearts. At peak performance the available HEP in 21° C. hearts was significantly greater than 4° C. hearts, and continued to be so during course of the experiments. In contrast, 4° C. hearts were unable to synthesize HEP to keep with the energy demands, thus HEP continued to decrease in the working hearts. These results are in agreement with previous observations that, unlike in hearts preserved in SOMAH at ambient temperatures, exposure to severe hypothermia leads to attenuation of HEP synthesis upon reperfusion in these hearts (Lowalekar S K, Lu X G, Thatte H S: Further evaluation of somah: long-term preservation, temperature effect and prevention of ischemia-reperfusion injury in rat hearts harvested after cardiocirculatory death. Transplant Proc. 2013, 45 (9): 3192-3197).

Antegrade perfusion was significantly lower in the 4° C. heart than in 21° C. hearts, and remained diminished, even at higher perfusion pressures until the system temperature stabilized at 37° C. (Table 4-1). Without being bound to theory, it is plausible that sudden shock of encountering 4° C. cardioplegia by the normothermic beating heart leads to profound vasoconstriction that does not resolve during storage and only does so upon initiation of reperfusion and raising of temperature to 37° C. and potentially because of active release of vasodilators nitric oxide and prostacyclins (Thatte H S. Rousou L, Hussaini B E, Lu X G, Treanor P R, Khuri S F: Development and evaluation of a novel solution. Somah, for the procurement and preservation of beating and non-beating donor hearts for transplantation. Circulation. 2009, 120: 1704-1713). Increased vasodilation, greater coronary vascular patency and a favorable metabolic status provides for rapid nourishment and H+ washout, resulting in robust synthesis of HEP and swift recovery of function in the 21° C. hearts. These hearts reverted to sinus rhythm with a single cardioversion and rapidly attained cardiac and hemodynamic parameters approaching in vivo range (Table 4-2), not requiring any inotropic support. On the other hand, 4° C. hearts demonstrated strong contraction only when warmed to 37° C. some of the hearts requiring additional electroversion and/or inotropic intervention, albeit ten times less than that reported in human hearts in vitro (Hill A J, Laske T G, Coles J A, Sigg D C, Skadsberg N D, Vincent S A, Soule C L, Gallagher B A, Iaizzo P A: In vitro studies in human hearts. Am Thorac Surg. 2005, 79: 168-177) to maintain cardiac output.

Release of cardiac enzymes was observed in both the groups upon reperfusion. An important mechanism of release of enzymes from the cardiomyocyte is by cytosol leakage during intracellular vesicular trafficking and incorporation (such as vesicles harboring glucose transporters; GLUT) into cell membrane, by a HEP dependent process, in response to external stimuli like insulin and increased metabolic demands in working hearts (Ferrera R, Benhabbouche S, Bopassa J C, Li B: One hour reperfusion is enough to assess function and infarct size with TIC staining in Langendorff rat model. Cardiovasc Drugs Ther. 2009, 23: 327-331). Therefore, release of cellular enzymes can occur even in absence of actual damage to the cardiomyocytes (enzyme paradox). Without being bound to theory, the initial burst of enzyme release in 21° C. cardioplegia group upon initiation of reperfusion likely resulted from greater availability of HEP at the end of storage for vesicular transport as the metabolic demands were ramped up with increase in system temperature and cardiac contractility (FIG. 17). However, upon reaching metabolic steady state any further release of enzyme was temporally attenuated. In contrast, in the 4° C. hearts, the rate of release of enzymes increased with time as more HEP became available for these functions. Even though the present data does not differentiate between progressions of enzyme release, the fact that the cardiac functional parameters in both groups were similar and approach the physiological values observed in vivo (Table 4-2) indicate that the releases of enzymes by the SOMAH hearts is a marker of metabolism rather than tissue injury and hence these hearts would perform well upon transplant.

### Example 5

This Example was designed to evaluate the novel storage solution Somah in its ability to maintain phosphate synthesis of livers and halt progression of long-term static storage-dependent multicellular damage. The aim of this current pilot study was to evaluate the comparative efficacy of Somah with the currently clinically used University of Wisconsin solution (UWS) in their ability to preserve and potentiate recovery of DCD porcine livers in vitro during a 72 hour period of hypothermic storage. A limited extracorporeal hepatic reperfusion and functional evaluation of Somah-stored livers was also performed to test the feasibility for future transplant studies to evaluate graft function.

Materials and Methods

CoStorSol (UWS) (Preservation Solutions Inc, Elkhorn, Wis.) and Somah (Somahlution, LLC, Jupiter, Fla.), Table 5-1, was compared for storage properties. All other chemicals were obtained from Sigma-Aldrich (SL Louis, Mo.). VetScan iStat, VetScan VS2, CG4+, CG8+, Large Animal Profile. Comprehensive Diagnostic Profile cartridges for measuring blood gases, electrolytes, lactate, glucose, aspartate transaminase (AST), alanine transaminase (ALT) and creatine kinase (CK) enzymes were purchased from Abaxis Inc (Union City, Calif.).

TABLE 5-1
Composition of Somah Organ Preservation solution (pH 7.5)
and University of Wisconsin Solution (UWS; pH 7.8).

| Components | Concentration (mM) | |
|---|---|---|
| Distilled water | 1.00 | L |
| Calcium | 1.30 | |
| chloride | 7.00 | |
| Potassium phosphate (monobasic) | 0.44 | |
| Magnesium chloride (hexahydrate) | 0.50 | |
| Magnesium sulfate (heptahydrate) | 0.50 | |
| Sodium chloride | 125.00 | |
| Sodium bicarbonate | 5.00 | |
| Sodium phosphate(dibasic; heptahydrate) | 0.19 | |
| D-glucose | 11.00 | |
| Glutathione (reduced) | 1.50 | |
| Ascorbic acid | 1.00 | |
| L-arginine | 5.00 | |
| L-citrulline malate | 1.00 | |
| Adenosine | 2.00 | |
| Creatine orotate | 0.50 | |
| Creatine monohydrate | 2.00 | |
| L-carnosine | 10.00 | |
| L-carnitine | 10.00 | |
| Dichloroacetate | 0.50 | |
| Insulin (10 mg/ml) | 1.00 | ml/L |

Composition of University of Wisconsin (UWS), (pH 7.8)

| Lactobionic Acid | 105 | |
|---|---|---|
| Potassium Dihydrogen Phosphate | 25 | |
| Potassium Hydroxide (56%) | 100 | |
| Sodium Hydroxide (40%) | 27 | |
| Magnesium Sulfate (heptahydrate) | 5 | |
| Raffinose (pentahydrate) | 30 | |
| Adenosine | 5 | |
| Glutathione | 3 | |
| Allopurinol | 1 | |
| Hydroxyethyl Starch (Pentafraction) | 50 | g/L |
| Insulin | 4.0 | mg/L |

Liver storage and procurement of samples. The study was conducted in fourteen female swines, each weighing 40-50 Kg in accordance with protocol approved by an Animal Studies Subconunittee (IACUC), VA Boston Healthcare System. The animals were divided into two groups of seven animals each. Whole livers were dissected out 60±10 minutes after cardiac death and extraction of heart. The livers were stored in UWS (UWS livers) or Somah solution (Somah livers) for 72 hours at 4° C. The solutions were not replaced during storage. Liver biopsies were obtained at 0, 6, 24 and 72 hours for imaging and biochemical assessment of viability. UWS and Somah solutions were sampled at 1, 6, 24 and 72 hours for metabolic monitoring and for other assays and compounds relevant to liver function.

Surgical procedure. General anesthesia was induced with i.m. injections of telazol 4-6 mg/kg and xylazine 2 mg/kg. After intubation, animals were maintained with i.v. propofol (10 mg/kg/hour), remifentanyl (40-60 μg/hr) and nimbex (cisatracurium) 10-20 mg, and mechanically ventilated. Aorta was cross-clamped, heart arrested, and heart-lung block was extracted for other experiments as described ((Lowalekar S K, Cao H, Lu X G, Treanor R, Thatte H S: Subnormothermic preservation in SOMAH: a novel approach for enhanced functional resuscitation of donor hearts for transplant. Am J Transplant 2014). After median laparotomy, suprahepatic aorta was cannulated, and abdominal organs lushed with 2 L of ice cold UWS or Somah solution at a pressure and flow rate of 100 mmHg and 300 ml/min respectively, till the perfusate returning through the suprahepatic inferior vena cava (IVC) was clear. The harvest was concluded with a total hepatectomy. Livers were transferred to plastic bags containing preservative solutions maintained at 4° C. in an icebox, and transported within 30 minutes to the lab for further analysis. Livers were transferred to storage box containing preservative solutions and stored at 4° C. for another 72 hours.

ATP and creatine phosphate assay. ATP and creatine phosphate (CP) were measured in liver tissue extracts. In brief, 20 mg of hepatic tissue was suspended in 400 μl of 0.4 M ice-cold perchloric acid and homogenized twice for 30 sec. Homogenate was centrifuged at 1970 g for 10 mins at 0° C. An aliquot of supernatant was neutralized with equal volume of ice-cold 0.4 M KHCO₁ solution and centrifuged as mentioned above. The supernatant was stored at −80° C. for ATP and CP measurements. The pellet was dissolved in equal volume of 0.1 M NaOH and centrifuged and used for protein assay. ATP and CP were measured using a bioluminescent assay kit (Sigma-Aldrich and GloMax-Multi+ Detection System, Promega) according to the protocol provided by manufacturer.

Preparation of blood for Ex vivo studies. Systemically heparinized blood was collected intraoperatively, leukodepleted and stored at 4° C. Prior to the commencement of experiments, the hematocrit was adjusted to 20% using Somah solution (now perfusate). The perfusate, pH, glucose, K+, Ca2+ and HCO₃— were adjusted for swine blood levels (7.5; 100 mg/dl; 3.7, 1.38, and 32 mmol/l respectively), using 10% dextrose, KCl, CaCl2 and NaHCO₃, respectively; gases were adjusted as required.

Ex vivo perfusion. Livers were stored in Somah for 72 hours at 4° C. (n=3). Hepatic artery and portal vein were identified and cannulated. The livers were kept in a polypropylene perfusion chamber attached to a custom built Somah Device used for ex vivo reanimation of hearts. An oxygenator, heat exchanger, clinical documentation improvement (CDI) monitor and data acquisition device with custom written software (Comdel inc. Wahpeton, N. Dak.) were incorporated into the system for real-time monitoring of changes in perfusate pH, temperature, pO₂, pCO₂, K+ and HCO₃-apop, pressure and flow rates. Somah device reservoir was filled with 2 L perfusate. Livers were gently flushed through the portal vein with 2 L of cold Somah, and then connected to Somah device via the hepatic artery (HA) and portal vein (PV). The reservoir outlet was diverted into two circuits: in the first circuit, the perfusate drained by gravity into the PV at a pressure of 8-10 mmHg (adjusted by changing the height of the reservoir). In the second circuit, perfusate was diverted through a pump to the HA (at pressures of 80-100 mmHg) via the oxygenator and heat exchanger. Temperature of perfusate was raised to 37° C. over a 20 min period and the perfusate was circulated through the liver for next 2 hours, the liver perfusate drained into a chamber through hepatic veins (HV) and was returned to the reservoir by another pump. Perfusate draining from the HV was temporally sampled for albumin, liver enzymes and other metabolites as mentioned below. Because of damage to the DCD livers stored in UWS, this functional assessment was performed only in Somah-livers.

Analysis of metabolites and liver enzymes. Blood parameters were assessed in perfusate inflow (HA) and outflow (HV). CDI monitor, VetScan iStat and VetScan VS2 were employed to determine biochemical parameters, blood gas, albumin synthesis and liver enzymes including alkaline phosphatase (ALP), Alanine aminotransferase (ALT). Aspartate aminotransferase (AST), γ-glutamyl transpepstidase (GGT) and creatine kinase (CK).

Histopathology. Tissue biopsies of livers stored either in Somah or UWS were taken at 0, 6, 24 and 72 hour storage time points, fixed in 10% formalin and processed for histopathology (10μ sections; Hematoxylin and Eosin stain). The images were acquired and analyzed using Olympus microscope and image analyzer system (BX51TRF; Olympus America Inc, USA). Images were assessed blindly for histopathology by three independent observers. Statistical analyses: The measurements and data extraction were performed in a blinded fashion. Comparison within the two groups (UWS vs. Somah, n=7 in each group; static storage) was conducted to evaluate the effect of the two solutions on organ function. The quantified initial values of various assays were compared to subsequent time points within each group using one-way analysis-of-variance (ANOVA), followed by Dunnett's multiple comparison test and t-test for comparative analysis between paired groups. Statistical significance was accepted at 95% confidence level (P<0.05). All values used were mean±SEM unless otherwise indicated. All analyses were performed using GraphPad Prism 6 (version 6.1), using a 0.05 significance level.

Results

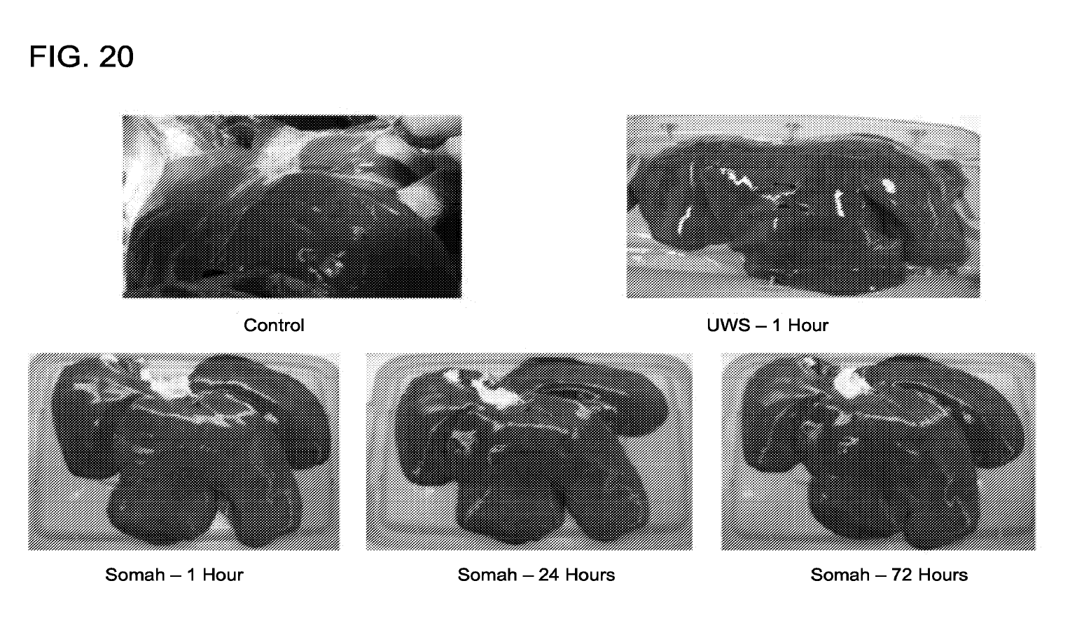

Gross appearance: Upon storage in UWS at 4° C., the gross appearance of livers was rapidly altered to a discolored state within the first hour of storage and subsequently continued to temporally deteriorate (not shown). In contrast, livers stored in Somah maintained their color similar to freshly extracted livers (control), even after extended 72 hour storage at 4° C. (FIG. 20). Furthermore, there was no weight gain by the Somah livers during storage (not shown)

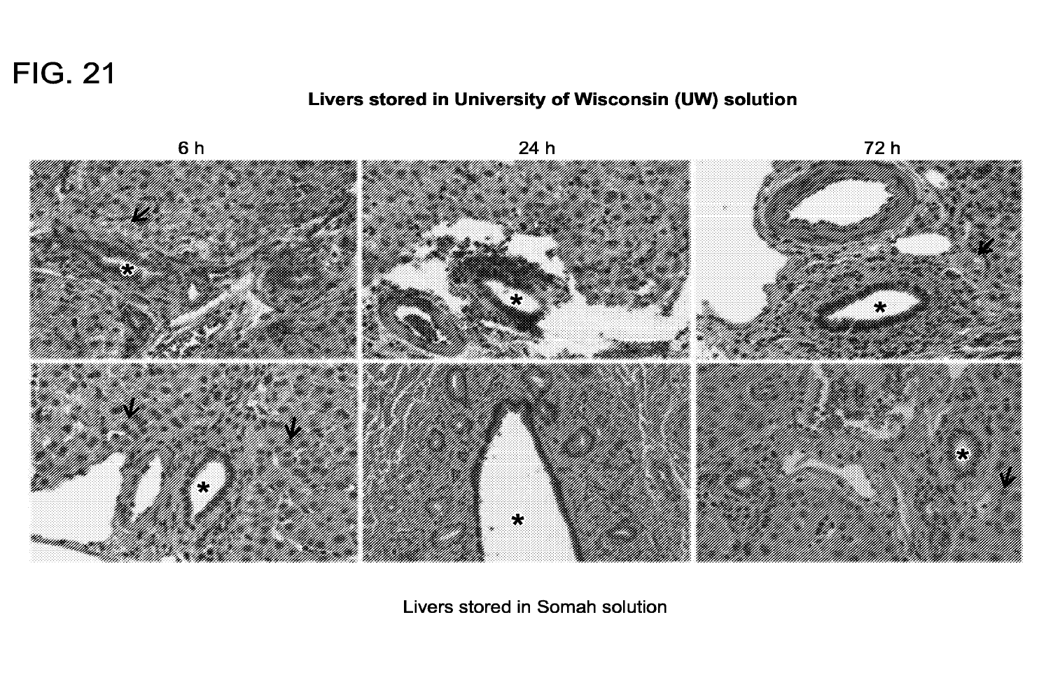

Hepatocytes: Nuclear chromatin condensation and pyknotic changes of hepatocytes in numerous low power fields were seen in livers stored in UWS but not in Somah. Binucleate and polyploid hepatocytic nuclei were consistently seen in sections obtained from both UWS and Somah-preserved livers. Furthermore, in Somah livers, cellular boundaries between adjacent hepatocytes were visibly intact, and no cholestasis or bile canalicular dilatations were apparent upon careful scan of multiple fields. In sections obtained from UWS livers at 72 hrs, degeneration of hepatocytes associated with extensive vacuolation was evident, but not in Somah livers (FIG. 21).

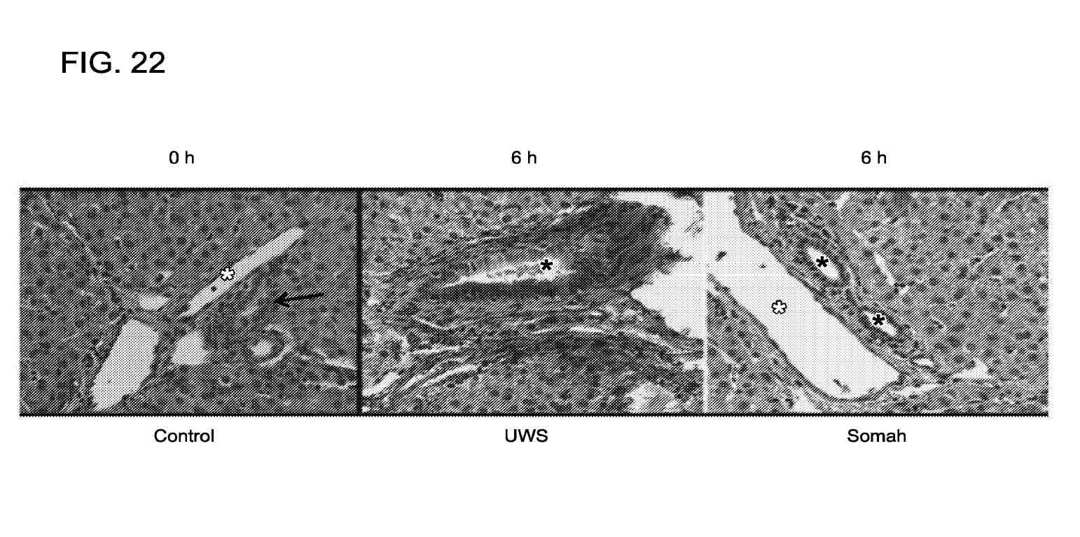

Biliary ducts and ductules: Nuclei lining both bile ductules and larger bile ducts in the portal triad appeared intensely pyknotic, suggesting apoptosis or necrosis of cholangiocytes in UWS livers. In contrast, the bile ducts and ductules were uniform in appearance with regularly placed nuclei and well preserved nuclear heterogeneity in Somah livers after 72 hrs of storage (FIG. 21). Furthermore, in UWS livers, there was denudation of bile ductular epithelium and sloughing of the mucosa along with disorganization of the basally placed nuclei of the ductules as early as 6 hr time point (FIG. 22). In contrast, such denudation of bile ductular epithelium was not visible in Somah livers even after 72 hours of storage (FIGS. 21 and 22).

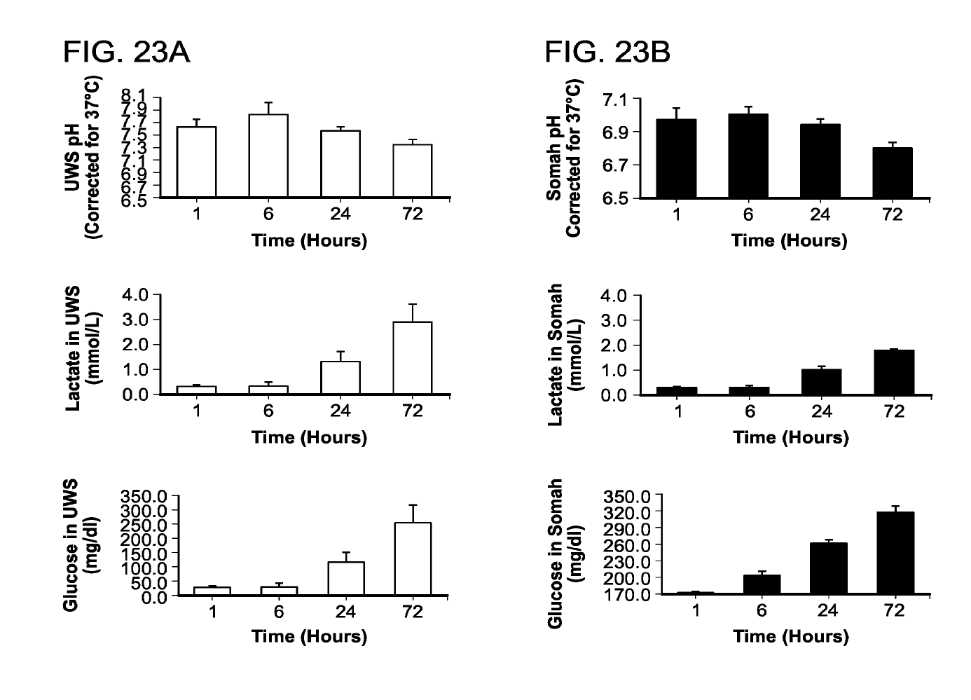

Metabolism in stored livers. While the starting pH for freshly reconstituted UWS and Somah was alkaline (UWS pH being higher than Somah), it was more acidic (6.8-7.0) in Somah compared to UWS (7.3-7.8) during all storage time points. There was a greater increase in lactate in UWS despite a lesser decrease in pH. Production of lactate did not correspond with sharp drop in solution pH, perhaps due to high buffering capacity of both the solutions. There was 1.5-fold increase in lactate levels in the UWS in comparison to Somah solution, which was significantly different at 24 and 72 hours, respectively (p<0.05) (FIGS. 23A and 23B).

As shown in FIG. 23 (lower panel), there was a comparable increase in glucose concentration in both UWS and Somah solution. Glucose concentration in UWS increased from 0 mg/dl to as high as 140 mg/dl at the end of 72 hour storage. Similarly, the glucose levels in Somah increased by 1.72 fold to 320 mg/dl over baseline levels of 180 mg/dl during the same time period, indicating greater glycogen breakdown than glucose reuptake. Oxidative phosphorylation is attenuated by hypothermia. Since in an open system, the solubility of atmospheric oxygen in water is inversely related to temperature, both Somah and UWS were supersaturated with oxygen at 4° C., with a pO₂ of 200±13 mmHg at the start of storage. Therefore, utilization of oxygen by the livers during extended temporal hypothermic storage cannot be clearly demonstrated, as oxygen consumption, if any, follows zero order kinetics.

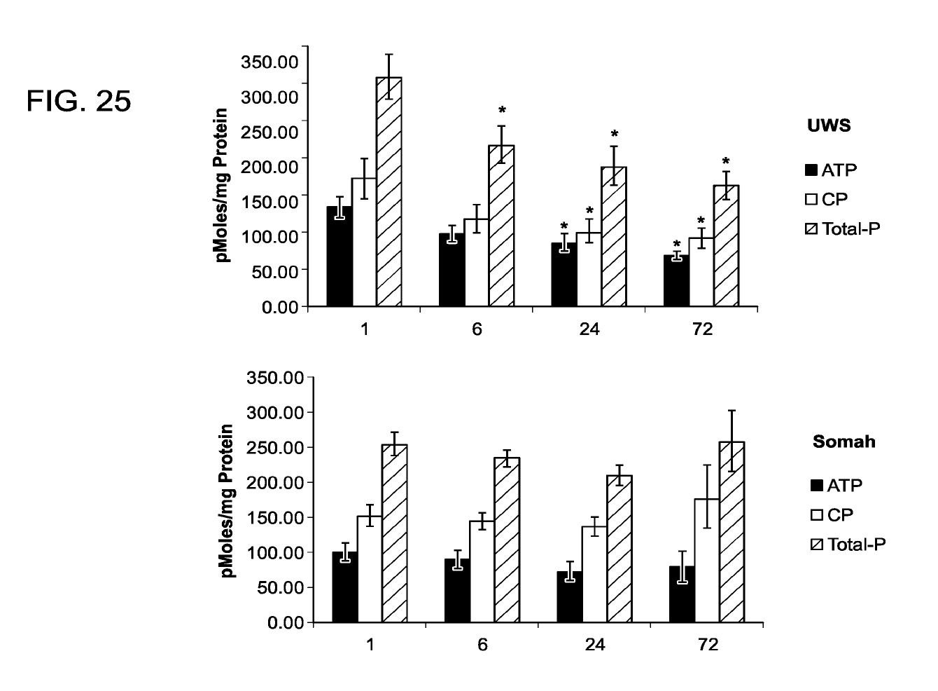

On the contrary, while the pCO₂, an indicator of dissolved CO₂, does not change in Somah or UWS solution in the absence of organs (not shown), the present studies demonstrated a significant increase in pCO₂ in Somah with stored liver during the 72 hour storage period, indicating oxidative metabolic turnover in Somah-stored DCD livers. In contrast, CO₂ levels were significantly lower and remained unaltered in UWS with stored liver during the 72 hour period. In UWS, pCO₂ measured at 30 minutes (6.30±0.38 mmHg) after the immersion of the liver and transfer to the lab increased insignificantly to only 7.33±0.48 mmHg over 24 hours and 9.27±0.89 mmHg at 72 hours storage. In contrast in Somah, the pCO₂ increased significantly from 10.8±1.13 mmHg within 30 minutes after initial immersion of the liver to 15±1.45 mm Hg at 1 hour, to 27±1.14 mmHg at the end of 72 hours, indicating temporal increase in metabolism. The greater pCO₂ in Somah than UWS at 30 minutes is indicative of active metabolism from the start of storage. Probability of bicarbonates present in Somah contributing to this increase in pCO₂ was negated by the fact that HCO₃— concentration remained mostly unaltered (4.23 mM/L) during the 72 hour storage. Conversely, HCO₃— concentration decreased from 7.30 to 5.33 mM/L in UWS during the 72 hour storage, which, without being bound to theory, may have contributed to the non-significant increase in pCO₂ that was observed in UWS during the storage period (FIG. 25). Another possibility to be considered is that anaerobic glycolysis, through lactate production, may have contributed to the rise of pCO₂ in Somah (as well as the mild rise in UWS stored livers). These data indicate that at least cellular metabolism was kept intact.

Measurement of phosphates in stored livers. ATP. CP and total phosphate concentrations decreased significantly (p<0.05) in UWS livers during the 72 hour storage (FIG. 25). The total phosphate decreased temporally in UWS livers during storage, resulting in 32% drop in the first 6 hours, followed by a 50% decrease by the end of 72 hours. In contrast, ATP, CP and total phosphate levels did not change appreciably in Somah livers during the entire course of preservation. An increase in total phosphate concentration at 72 hours in Somah livers was observed (FIG. 25), analogous with metabolic production of CO₂.

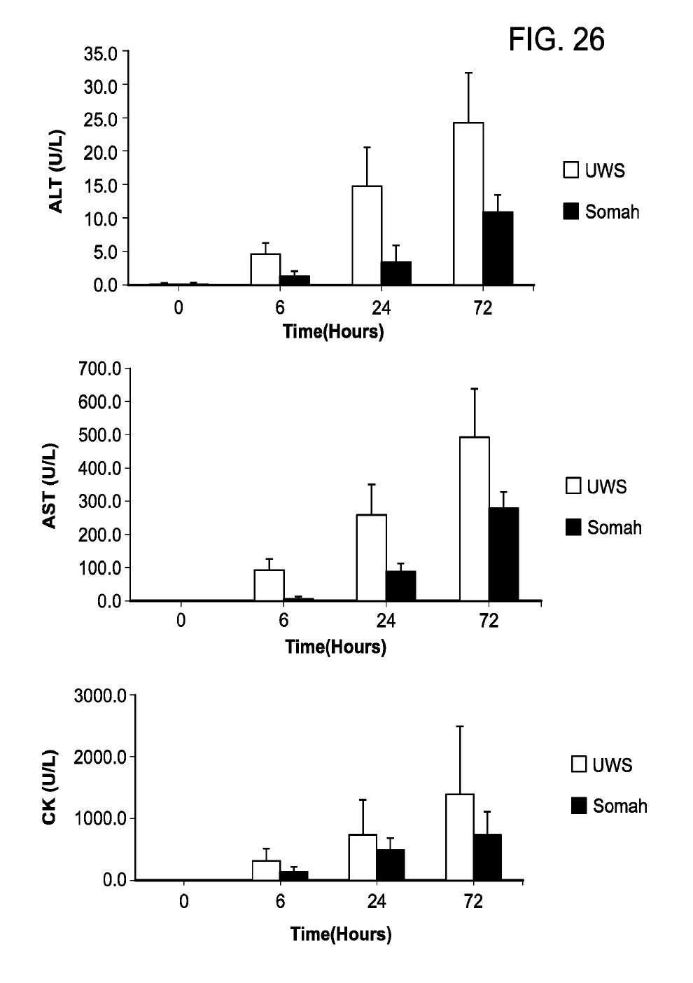

Release of Liver Enzymes during Storage. A time dependent release of ALT, AST and CK enzymes was observed in storage solutions as markers of liver tissue injury. Release of all three enzymes was significantly elevated (P<0.05) when livers were stored in UWS, in comparison to livers stored in Somah (FIG. 26).

Functional evaluation of somah livers. Somah stored livers (72 hours) were reperfused with blood (perfusate) for 2 hours at 37° C. for functional evaluation. Oxygen consumption by the reperfused livers increased significantly by 38% and 64% (p<0.05) at 30 and 120 min, respectively. Concomitantly, lactate concentration released in the perfusate decreased by 11% and 41% at 30 min and at the end of 2 hours, indicating anaerobic to aerobic metabolic switch.

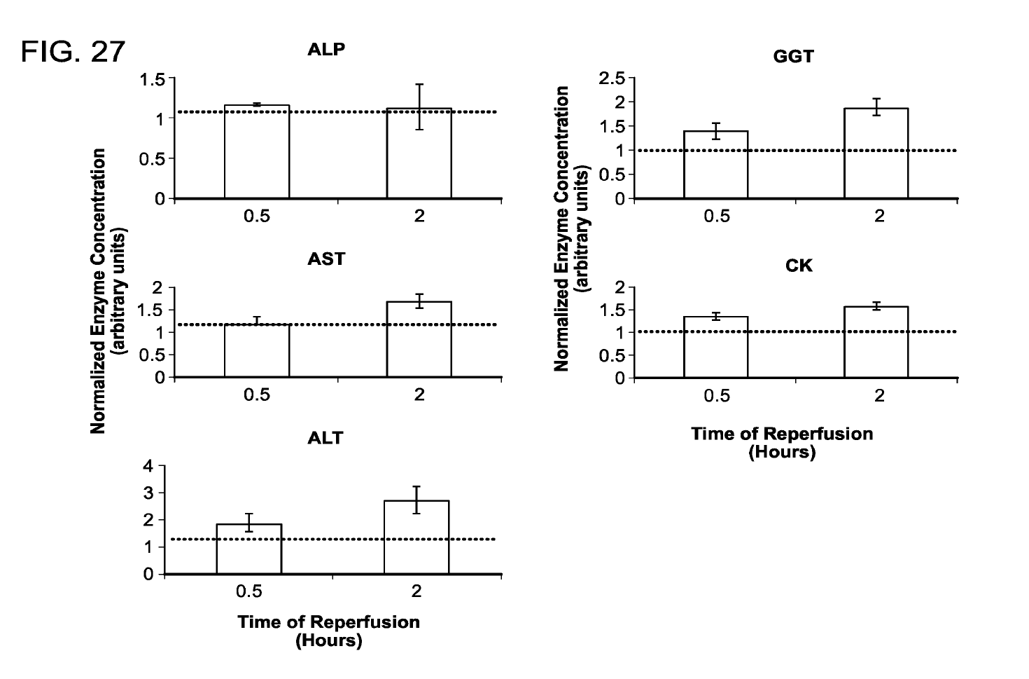

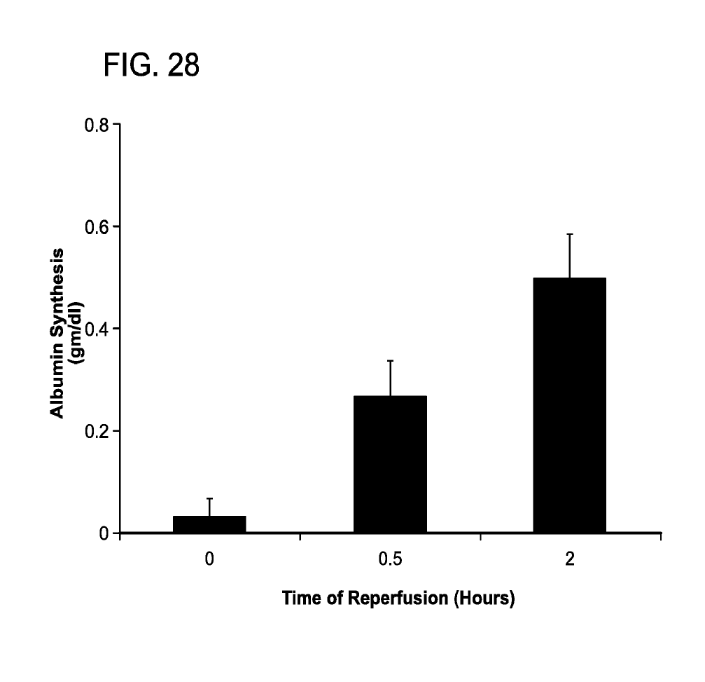

There was no significant difference in temporal release of liver enzymes during reperfusion (FIG. 27). Both ALP (15.33±0.96) and γ-GT (17.5±1.06 U/L) were within physiological concentrations at the end of 2 hours. However, AST (2151±81), ALT (228±32) and CK (1428±205 U/L) concentrations were elevated in the perfusate at the end of reperfusion. There was a significant increase in synthesis and release of albumin by the Somah livers within 30 min after reperfusion (p<0.03), which increased temporally over the 2 hour period (p<0.01) (FIG. 28). Synthesis and release of bile was also increased temporally in perfused Somah livers (data not shown).

The objective of this Example was to compare “Somah” and UWS solution for extended temporal extracorporeal preservation of porcine DCD livers. In this study, evidence of efficient storage properties with of Somah over UWS to functionally salvage DCD livers is provided. Without being bound to theory, it was hypothesized that the unique formulation of Somah temporally maintains and/or augments energy state of an organ during extracorporeal storage, resulting in enhanced cellular homeostasis and structural integrity, thereby, effectively improving the overall repair and recovery of excised organ during the storage period.

The results of the present study reveal that progressive histological injury of DCD livers is potentially preventable, depending upon the composition of storage solution. The karyopyknosis in hepatocytes and reactive changes of biliary epithelial cells were visible as early as 6 hrs in UWS-livers, but not in Somah-livers at 72 hour time point. Furthermore, this study provides detailed findings of different kinds of biliary injury, earlier only scantily reported (Kochhar et al., 2013, World J. Gasterenterol., 19:2841-46). DCD livers stored in UWS showed degenerative changes in both small and large bile ducts, a potential cause of non-anastomotic biliary stricture and biliary dysfunction.

This has been reported to result in poor post-transplant graft functioning (Kochhar et al., 2013. World J. Gasterenterol., 19:2841-46) and greater morbidity (Yan et al., 2011, J. Surg. Res., 169:117-124). Without being bound to theory, the higher K+ levels and ischemia may have augmented cellular damage in UWS-livers (Lowalekar S K, Cao H, Lu X G, Treanor P R. Thatte H S. Subnormothermic preservation in somah: a novel approach for enhanced functional resuscitation of donor hearts for transplant. Am J Transplant. 2014; 14:2253-62). Furthermore, while acidic pH is reported to be constitutively beneficial to hepatocytes and sinusoidal epithelial cells (Lowalekar S K, Cao H, Lu X G, Treanor P R, Thatte H S: Sub-normothermic preservation of donor hearts for transplantation using a novel solution. SOMAH: a comparative pre-clinical study. J Heart Lung Transplant. 2014, 33 (9): 963-970), the pH of Somah, compared to UWS remained relatively acidic throughout the preservation period. The enzyme assay studies showed significantly diminished hepatocellular injury of livers stored in Somah in contrast to UWS.

While during hypothermic perfused organ storage, glycolysis appears to be the primary energy source at a pO₂ of 150 mmHg (Opie L H, Lopaschuk G D (2004) Fuels: aerobic and anaerobic metabolism. In: Opie L H, ed. Heart Physiology: From Cell to Circulation. 4th ed. Philadelphia, Pa.: Lippincott, Williams and Wilkins 306-354), the dichloroacetate (DCA) in Somah likely diverts the pyruvate generated by glycolysis into Krebs cycle, thus further enhancing ATP synthesis and maintenance of phosphates (FIG. 25). Furthermore, the DCA, by enhancing oxidative metabolism of pyruvate, also prevents build-up of lactate in Somah stored livers. Moreover, insulin, which enhances the entry of glucose into cells, is a hepatotrophic factor and is essential for maintenance of hepatic ultrastructure and regenerative ability. Somah solution exploits this ability of insulin by providing it in a concentration of 100 U/L, 2.5 folds higher than that in UWS. Thus, the greater lactate accumulation above threshold levels in the absence of DCA and the lower insulin concentration in UWS-livers contributes to the comparative changes in this group.

Loss in phosphates in the explanted organs during storage leads to irreversible degenerative changes in the organ. Despite an equivalent increase in glycogenolysis-dependent glucose concentration in both UWS and Somah solution livers during storage, there was a depletion of phosphate stores in UWS, in contrast to enhancement in Somah. Without being bound to theory, this suggests that UWS livers are in fact in a catabolic state, resulting in loss of phosphates. In contrast, by promoting oxidative phosphorylation of glucose in Somah-livers, a 15 folds greater amount of phosphates are generated (for equivalent glucose molecules) than by anaerobic glycolysis alone (Brown, Biochem J. 1992, 284:1-13). Furthermore, during hypothermic perfusion, livers having greater ATP levels demonstrate lower oxidative stress upon re-warming (Belzer F, Southard J H, Transplantation. 1988 April; 45(4):673-6).

Static organ preservation has always been recognized as critical component in maintaining liver explants prior to transplantation (Pegg et al., Translpantation, 1981, 32:437-43). Limited studies have addressed the deleterious effect of storage of livers in UWS solution (Startzl et al., Hepatology, 2010, 5:1869-84). However, this does not appear to occur in DCD livers preserved in Somah. Preliminary functional studies indicate that there is a rapid switchover to aerobic from anaerobic metabolism, supported by similar observations in the heart (Lowalekar S K. Cao H, Lu X G. Treanor R, Thatte H S: Subnormothermic preservation in SOMAH: a novel approach for enhanced functional resuscitation of donor hearts for transplant. Am J Transplant. 2014). Similarly, there was a significant increase in synthesis of albumin, and release of bile in the reperfused Somah livers, indicating that metabolism and function are preserved after prolonged storage. UWS-stored livers were not evaluated for functional recovery because of gross damage to these organs.

The biliary system in Somah livers remains patent and intact, demonstrating lack of frank damage. It is well known that hepatocytes can repair or regenerate, especially if the energy state of the organ can be preserved. It is the failure of biliary system that leads to PNF and DGF (Kochhar et al., 2013. World J. Gasterenterol., 19:2841-46). Biliary dysfunction may be prevented by ex vivo storage in Somah.

### Example 6

Eighty-six of every hundred patients requiring renal transplant last year, did not get one, adding to the ever mounting waitlist for transplantrequiring individuals [National Kidney Foundation; http://www.kidney.org]. This is despite tremendous progress in solid organ transplantations in last several decades and availability of renal replacement therapy (Hemodialysis and Peritoneal dialysis) which are not only associated with severely debilitating and/or life-threatening complications but hinder routine lifestyle of patients due to frequent requirements for hospital visits, imparting a huge financial burden on society.

In 2015, 14000 patients in US received renal transplant, 2500 patients are added to renal transplant waitlist each month, with around 100,000 patients awaiting kidney transplantation at the time of writing this paper; the average waiting time being three to five years. Although, 65% of renal grafts are procured from deceased (DCD; Donation after Cardiac Death) donors, these grafts are twice as likely to develop delayed graft function (DGF) compared to standard-criteria donors, an increased incidence of primary non-function (PNF), and halved overall graft survival. Whereas grafts exposure to warm and/or cold ischemia is an obligate inevitability of solid organ transplant, the resulting cellular/tissue damage during this period is consequential of the less than desired post-transplant operative renal function. The above data indicate an enormous potential to improve quality of DCD kidneys for transplantation by making advances in preservation technology, and thus more desirable long-term patient outcomes.

This Example evaluated the ability of novel organ preservation solution Somah compared to University of Wisconsin (UW) solution for extended storage of DCD kidneys.

Materials and Methods

Surgical procurement of kidneys. Female Yorkshire Swine weighing 40-50 Kgs were used as per protocol approved by institutional animal studies committee. Swine were sedated with telazol 4-6 mg/kg i.m. and xylazine 2 mg/kg i.m., intubated and connected to ventilator. Anesthesia was maintained using i.v. propofol (10 mg/kg/hr) and remifentanyl (40-60 μg/hr). Cis-atracurium (10-20 mg i.v.), a paralytic agent, was administered ten minutes prior to surgery. Upon midline sternotomy, animals were systemically heparinized (300 mg/Kg) and aortic root cannulated. Ice cold cardioplegia (20 mM K+) was infused after aortic clamping to stop the heart which was then excised for other experiments as described [6,7]. Time of complete cessation of heart contraction was recorded as the beginning of warm ischemia of other body organs. Post median laparotomy, suprahepatic aorta was cannulated and abdominal organs flushed with 2 L of ice cold UW (CoStorSol; Preservation Solutions Inc., Elkhorn, Wis.) or Somah solution (Somahlution Inc., Jupiter, Fla.), at 100 mmHg pressure and a flow of 300 ml/min, till perfusate returning through inferior vena cava (IVC) was clear. Abdominal organ harvest was concluded first with hepatectomy for use in other experiments, and subsequent total bilateral nephrectomy after carefully dissecting the renal pedicles. Kidneys were immediately transferred to Somah or UW solution (Table 6-1) at 4° C. and static stored for 72 hours. Kidney biopsies were obtained for histopathology, HEP and Western blot assays at time 0, and 6, 24 and 72 hour time-points. Time 0 corresponds to 1 hour in storage; time required to transport kidneys from animal research facility to lab before first biopsy.

TABLE 6-1
Composition of Somah and UW solutions.
SOMAH (pH 7.5)
mmol/L*

| | |
|---|---|
| Potassium Phosphate (monobasic) | 0.44 |
| Potassium Choloride | 7.00 |
| Sodium Chloride | 125.00 |
| Sodium Bicarbonate | 5.00 |
| Calcium Chloride | 1.30 |
| Sodium Phosphate (dibasic; heptahydrate) | 0.19 |
| Magnesium Chloride (hexahydrate) | 0.50 |
| Magnesium Sulfate (heptahydrate) | 0.50 |
| D-Glucose | 11.00 |
| Glutathione (reduced) | 1.50 |
| Ascorbic Acid | 1.00 |
| L-Arginine | 5.00 |
| L-Citrulline Malate | 1.00 |
| Adenosine | 2.00 |
| Creatine Orotate | 0.50 |
| Creatine Monohydrate | 2.00 |
| L-Carnosine | 10.00 |
| L-Carnitine | 10.00 |
| Dichloroacetate | 0.50 |
| Insulin | 10 mg/L |

UW SOLUTION (pH 7.4)

| | |
|---|---|
| Lactobionic Acid | 105.00 |
| Potassium Dihydrogen Phosphate | 25.00 |
| Potassium Hydroxide (56%) | 100.00 |
| Sodium Hydroxide (40%) | 27.00 |
| Magnesium Sulfate (heptahydrate) | 5.00 |
| Raffinose (pentahydrate) | 30.00 |
| Adenosine | 5.00 |
| Glutathione | 3.00 |
| Allopurinol | 1.00 |
| Hydroxyethyl Starch (Pentafraction) | 50 g/L |
| Insulin | 4.0 mg/L |

Histopathology. Tissue was fixed in formalin and embedded in paraffin before cutting 10μ thin sections that were melted onto glass slides for further processing. Tissue sections were dried in sequentially increasing ethanol concentrations and then subjected to hematoxylin and eosin staining after which slides were immersed in xylazine clearing agent, covered with cover slip and examined under microscope. Images were acquired and analyzed using Olympus microscope and image analyzer system (BX51TRF; Olympus America Inc. USA) and assessed in blind fashion by independent observers.

ATP and creatine phosphate assay. ATP and creatine phosphate (CP) were measured in kidney tissue extracts as described [4-7,10]. In brief, 20 mg of renal tissue was suspended in 400 μl of 0.4 M ice-cold perchloric acid and homogenized twice for 30 secs. Homogenate was centrifuged at 1970 g for 10 mins at 0° C. An aliquot of supernatant was neutralized with equal volume of ice cold 0.4 M KHCO₃ solution and centrifuged as above. Supernatant was stored at −80° C. for ATP/CP measurements. The pellet was dissolved in equal volume of 0.1 M NaOH and centrifuged and used for protein assay. ATP/CP was measured using a bioluminescent assay kit (Sigma-Aldrich and GloMax Multi+Detection System. Promega) according to manufacturer's protocol.

Western blotting. 20 mg of kidney tissue was suspended in extraction buffer containing a protease inhibitor cocktail. Tissue was homogenized for 30 sees, centrifuged at 16,100×g for 10 mins and supernatant was collected. Equal amounts of total protein (30 μg) from different samples were mixed with Laemmli sample buffer containing 5% β-Mercaptoethanol, and heated at 100° C. for 3 mins. Proteins were resolved on 10% SDS-PAGE, and electro-blotted onto nitrocellulose membrane; proteins were identified using antibodies (anti-Caveolin, eNOS, vWF, and EPO) and chemiluminescence assays and band densities were normalized to betaactin as described [4].

Metabolic analysis. Changes in pH and lactate, glucose metabolism, oxygen and carbondioxide concentrations (pO₂ and pCO₂) in Somah and UW were assessed at 0 (1 hour, vide supra), 6, 24 and 72-hour time-points during storage using VetScan iStat and VetScan VS2.

Statistical analysis. The measurements and data extraction were performed in blinded fashion. Comparison within two groups (UW vs. Somah, n=7 in each group) was conducted to evaluate effects of the two solutions. Quantified initial values of various assays were compared to subsequent time points within each group using one-way analysis-of-variance (ANOVA), followed by Dunnett's multiple comparison test, and t-test for comparative analysis between groups. Statistical significance was accepted at 95% confidence level (p<0.05). All values used were mean±SEM unless otherwise indicated. All analyses were performed using GraphPad Prism 6 (v6.1). The authors had access to and take full responsibility for integrity of data. All authors have read and agree to the manuscript as written.

Results

Gross morphology of kidneys. Kidneys were examined during 1-3 days of immersion static storage at 4° C. Kidneys stored in UW showed dusky and mottled appearance (FIG. 1a), indicative of organ congestion. In contrast, kidneys stored in Somah appeared healthy, of uniform color and morphologically unaltered after 3 days storage (FIG. 1d).

Histomorphology of kidneys. Irrespective of storage solution, there was no evident interstitial edema in all DCD kidneys at observed time-points, with well preserved overall structure of renal tissue (FIGS. 1b,1c,1e and 1f). The normal amorphous collection in tubular lumen was observed in proximal convoluted tubules (PCT) at all time-points and not increased with storage duration. Distal convoluted tubules (DCT) remained mostly clear of any debris at all time-points except at 72 hour where a minimal to moderate epithelial denudation was apparent in both UW and Somah-preserved kidneys (FIGS. 1c and 1f). Renal glomeruli exhibited normal cellularity with normal appearing Bowman's space and continuous parietal epithelium at all time-points, in both solutions (FIGS. 1b,1c,1e and 1f).

A gradual time-dependent loss of tubular epithelial nuclear heterogeneity with increased hyperchromaticity and occasional loss of cellular margins was observed in both UW and Somah-stored kidneys, indicative of tubular epithelial injury (FIGS. 1c and 1f). However, extent of these changes were significantly greater in UW-stored kidneys (p<0.05). On an average 3.5%, 24.4%, 39.7% and 37% in UW kidneys, and 4.4%, 6.2%, 10.9% and 11.6% in Somah kidneys, of the epithelial cell nuclei (of PCT/DCT's) were severely hyperchromatic, at time 0, and 6, 24 and 72 hour storage time-points, respectively, suggesting that renal tubular epithelial cells in Somah-stored DCD kidneys were able to endure ischemia for a longer duration than UW-stored kidneys.

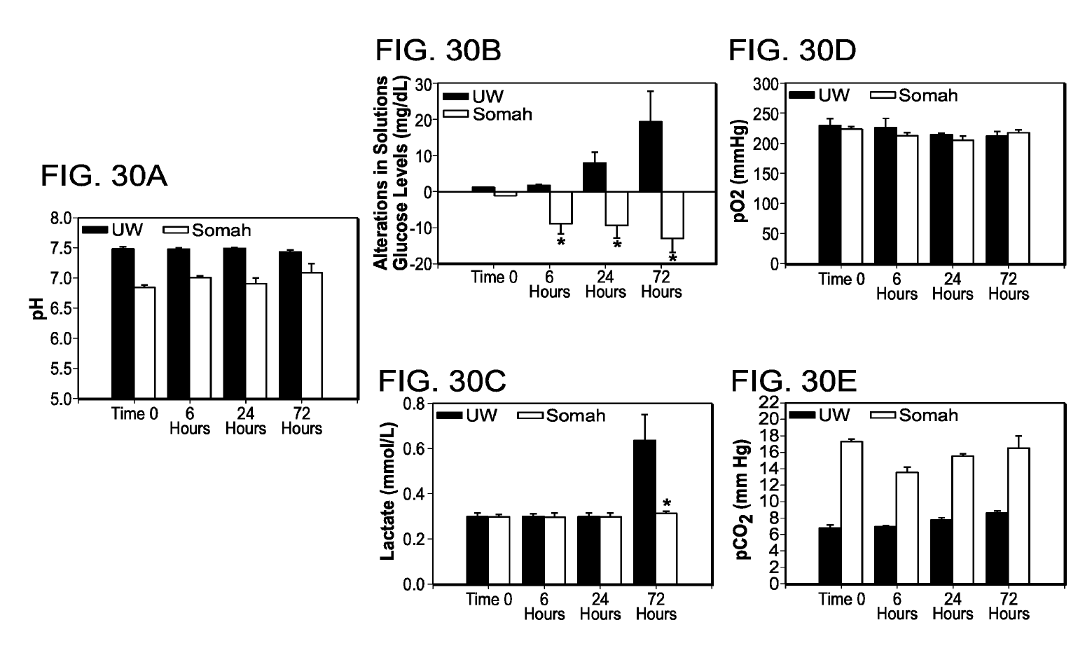

Metabolism in stored kidneys. DCD kidneys were evaluated for physiological/biochemical viability by assessing metabolic functions during extracorporeal storage; demonstrating differentially active metabolism in UW and Somah groups. While freshly reconstituted solutions started off with a more alkaline pH (moreso in UW), a time-dependent fall in pH was apparent in Somah, but remained more acidic (6.8-7.2) compared to UW (7.5-7.4) (FIG. 30A). While there was a temporal increase in glucose levels in UW solution, suggesting glycogenolysis, there was a comparative significant fall (p<0.05) in glucose levels in Somah, at and beyond 6 hour time-points, suggesting utilization of glucose present in Somah by kidney tissue/cells (FIG. 2B). Inversely, compared to Somah, there was a significantly greater (p<0.05) rise in lactate levels in UW at 72 hour time-point (FIG. 30C).

Since in an open experimental system as ours, the solubility of atmospheric oxygen in solution is inversely related to temperature, and since oxygen consumption follows zero-order kinetics, and as Somah and UW both were supersaturated with oxygen at 4° C., with a pO2 of 200±13 mmHg at all times (FIG. 30D), oxygen utilization by kidneys could not be clearly demonstrated. However, in contrast to UW, in which pCO₂ was significantly lower than in Somah throughout storage (7.28±0.40 mmHg at 1-hour and 7.50±0.48 mmHg at 72 hours), a significant initial increase in pCO₂ was observed in Somah (p<0.01) (from 5.8±1.15 mmHg after initial immersion of kidney to 17.00±0.45 mmHg) within 1 hour of DCD kidney storage, and remained consistently high during 72 hour period, indicating oxidative metabolic turnover right from the start of storage period (FIG. 30E). It must be noted that pCO₂, an indicator of dissolved CO₂, does not change in Somah or UW solution in the absence of organs (not shown). Probability of bicarbonates present in Somah contributing to increase in pCO₂ was negated by the fact that HCO₃— concentration remained mostly unaltered (4.86 mM/L) during 72 hour storage. Conversely, HCO₃— concentration decreased from 6.30 to 4.33 mM/L in UW during 72 hour storage that may have contributed to the non-significant increase in pCO₂ (FIG. 30E).

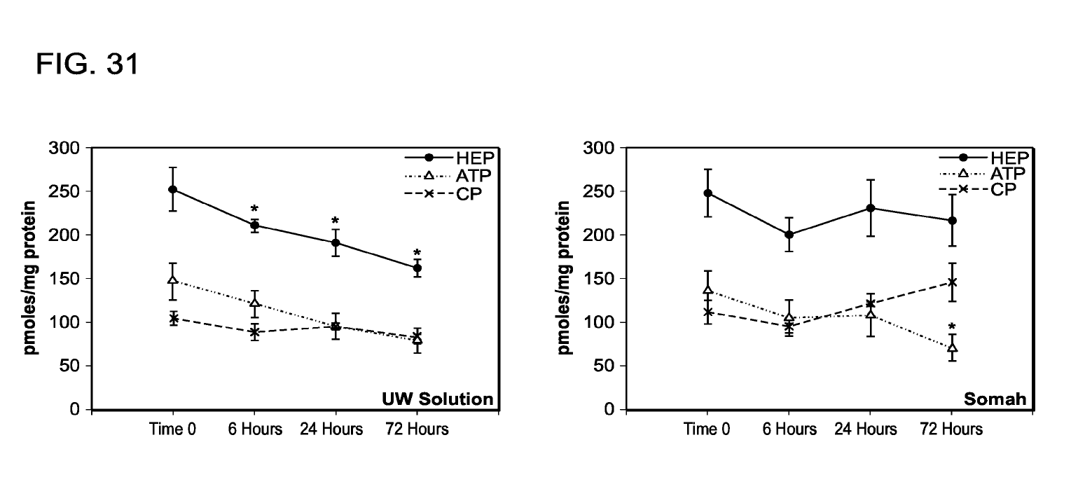

High-energy phosphates in stored kidneys. The UW kidney tissue ATP. Creatine Phosphate (CP) and total HEP concentrations decreased linearly and significantly (p<0.05) during hypothermic storage. HEP dropped by 20% within six hours (FIG. 31), with a net decrease of 45% at the end of 72 hour storage. In contrast, ATP, CP and total HEP levels did not change appreciably in Somah kidneys, and any decrease in ATP was compensated with a parallel increase in CP concentration, thus maintaining superior total energy levels in renal tissue during storage.

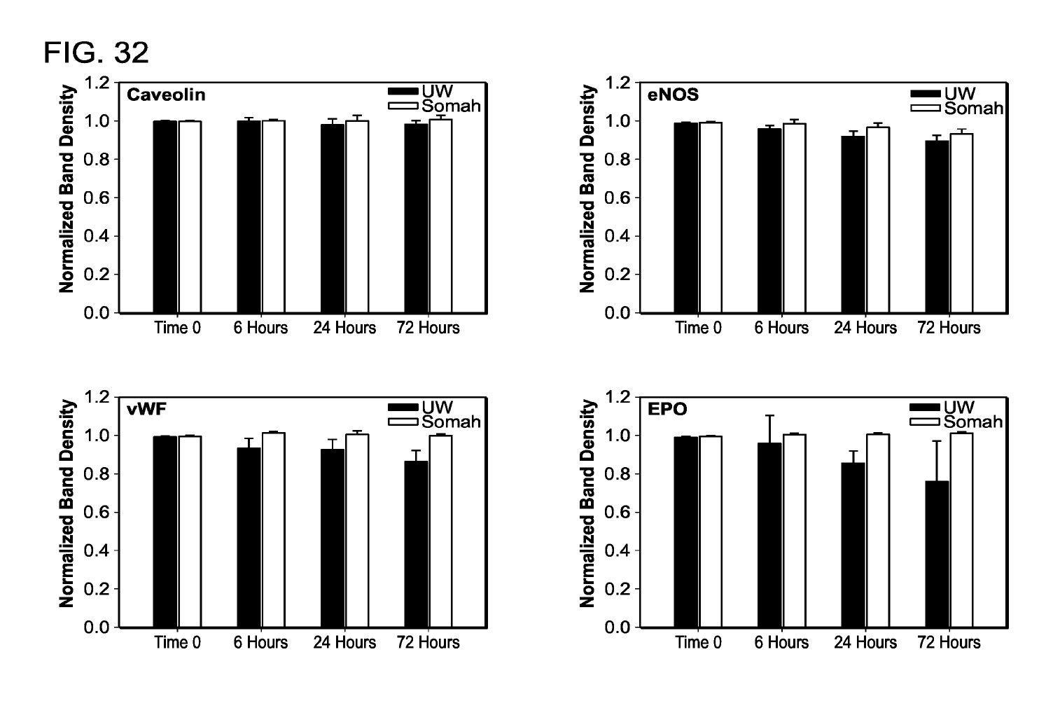

Markers of vascular endothelial function. The expression of caveolin, eNOS, vWF and EPO was well preserved in DCD kidneys preserved in Somah, during the entire storage period (FIG. 32). In contrast, while expression of caveolin protein was unaltered in UW-preserved kidneys as well, there was time-dependent decrease in expression of eNOS, vWF and EPO, indicative of possible renal tissue damage.

Being one of the most resistant internal organs to ischemia, use of kidneys from deceased (DCD) donors for transplantation has been commonly practiced worldwide (Being one of the most resistant internal organs to ischemia, use of kidneys from deceased (DCD) donors for transplantation has been commonly practiced worldwide (Morrissey P E, Monaco A P (2014) Donation after circulatory death: Current practices, ongoing challenges, and potential improvements. Transplantation 97: 258-264). While DCD pool of kidneys is still highly underutilized, prognosis of DCD kidneys upon transplantation is associated with an observably increased incidence of DOF, PNF and lowered graft life expectancy in recipients. Without being bound to theory, the results of the present study suggest that by maintaining energy levels in DCD kidneys during storage using Somah, subtle damage at cellular levels can be avoided.

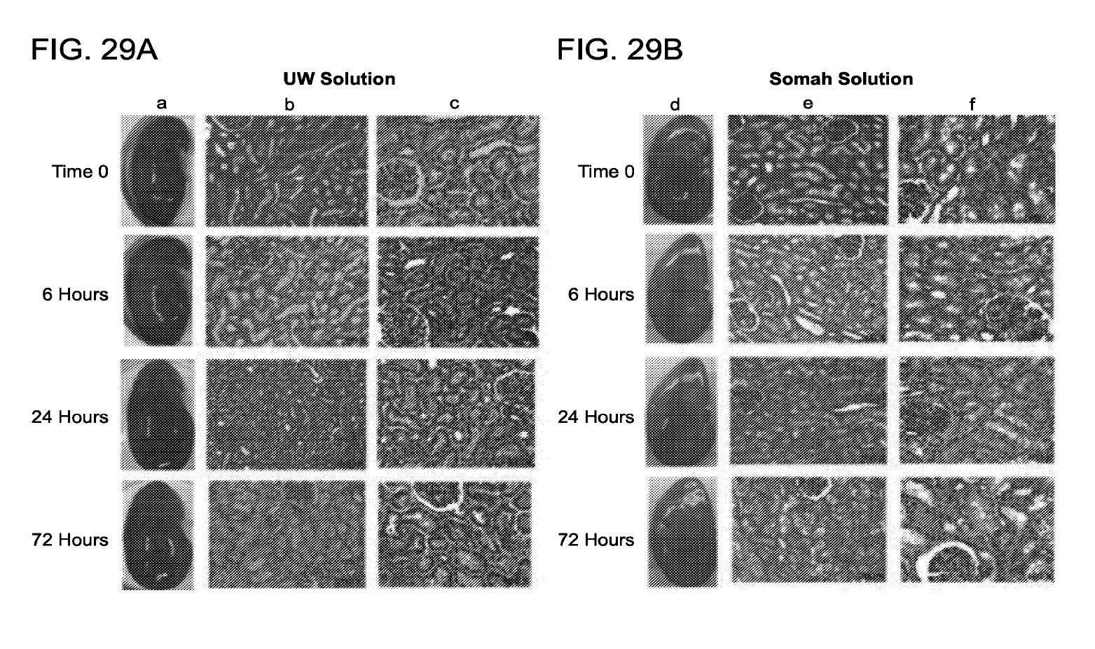

The results show that UW-preserved kidneys display patchy areas of discoloration within a few minutes of organ perfusion with UW, upon harvesting, and did not resolve during 72 hour storage. In contrast, Somah-preserved kidneys remained uniform in their external morphology at all time-points. While Somah has a viscosity close to that of normal saline, UW is characterized by a much higher viscosity due to hydroxyl-ethyl starch (HES: Table 6-1) (Collins G M, Wicomb W N (1992) New organ preservation solutions. Kidney Int Suppl 38: S197-S202). While HES helps to prevent organ edema during storage, it increases solution density that can interfere with perfusion of all parts of the organ. Although gross morphology is not the best indicator of organ viability, it is probable that an uneven perfusion of kidneys during harvesting, despite copious use of solution for perfusion, could have resulted in patchy discoloration of kidneys, due to greater UW viscosity (FIG. 29).

Renal histopathology showed no gross ultrastructural changes in either cortical or medullary regions of UW or Somah-stored kidneys. However, higher magnifications revealed subtle changes in cellular nuclei, especially in tubular epithelial cells, characterized by loss of nuclear heterochromacity with increased hyperchromacity, significantly greater in UW-stored kidneys. This is consistent with an inadequacy of UW to reach all parts of kidneys (vide supra), while Somah reached effectively and provided necessary nutrients to tissues in their entirety during harvesting and extracorporeal storage, thus avoiding development of minor lesions and may potentially improve the post-transplant outcomes. Despite better endurance of renal tissue in Somah, Somah pH was more acidotic, compared to UW, at all time-points. While acidic pH is reported to be constitutively beneficial to hepatocytes, sinusoidal epithelial cells as well as cardiomyocytes (Lemasters J J, Bond J M, Currin R T, Nieminen A L, Caldwell-Kenkel (1993) Reperfusion Injury to Heart and Liver Cell: Protection by acidosis during ischemia and a ‘pH paradox’ during reperfusion. In: Hochachka P W, Lutz P L, Sick TJ, Rosenthal M (eds) Surviving Hypoxia: Mechanisms of Control and Adaptation. CRC Press, Inc, Florida 495-508), this is the first report showing advantage of a relatively acidic environment in extracorporeal kidney preservation.

Glucose, an important energy source for highly metabolic renal tissue, was excluded from kidney preservation solutions used in current practice as it was thought to cause edema by enhanced lactate accumulation, a product of anaerobic metabolism during extracorporeal storage (Kallerhoff M, Holscher M, Kehrer G, Klab G, Bretschneider H J (1985) Effects of preservation conditions and temperature on tissue acidification in canine kidneys. Transplantation 485-489). UW, a commonly used solution for renal storage, does not contain any glucose either. However, that glucose is an important energy source during extracorporeal renal storage was demonstrated by an endogenous increase in glucose levels in UW, likely due to renal glycogenolysis, and a corresponding fall in glucose levels in Somah, which innately contains high concentration of glucose (Table 6-1). However, even after prolonged storage of kidneys in Somah, development of edema was not observed by gross or histological examination, contrary to previous studies involving other solutions (Kallerhoff M. Blech M. Kehrer G, Kleinert H, Langheinrich M, et al. (1987) Effects of glucose in protected ischemic kidneys. Urol Res 15: 215-222).

After 72 hour kidney storage, UW demonstrated substantial rise in deleterious lactate levels that remained low at all time-points in Somah (FIG. 30C), despite metabolism (anaerobic and aerobic) of glucose, leading to corresponding maintenance of tissue HEP in Somah kidneys (FIG. 31). Aerobic oxidative phosphorylation activity in Somah kidneys was confirmed by observed increase in metabolic CO₂ in Somah during storage. This was an expected result; as Somah also contains dichloroacetate (DCA), a compound that increases the activity of pyruvate dehydrogenase complex thus enhancing conversion of pyruvate to acetyl-CoA, preventing accumulation of lactate (Shangraw R E, Winter R. Hromco J, Robinson S T, Gallaher E J (1994) Amelioration of lactic acidosis with dichloroacetate during liver transplantation in humans. Anesthesiology 81: 1127-1138) confirming observations in hearts and livers stored in Somah (Thatte H S, Rousou L. Hussaini B E, Lu X G, Treanor P R, et al. (2009) Development and evaluation of a novel solution, Somah, for the procurement and preservation of beating and non-beating donor hearts for transplantation. Circulation 20: 1704-1713). Furthermore, without being bound to theory, DCA, by virtue of its vessel-preservation abilities, may help prevent development of post-transplant renal artery stenosis, and further improve prognosis of transplanted DCD kidneys (Deuse T, Hua X, Wang D, Maegdefessel L. Heeren J, et al. (2014) Dicholoroacetate prevents restenosis in preclinical animal models of vessel injury. Nature 509: 641-644).

A significant loss of energy state (HEP) in explanted organs during storage leads to irreversible degenerative changes in the organ (Vajdova K, Graf R, Clavien P A (2002) ATP-supplies in the coldpreserved liver: a long-neglected factor of organ viability. Hepatology 36: 1543-1551). It is thus imperative for preservative solution to maintain organ in homeostasis and/or allow it to recover during extended storage by modulating organs metabolic pathways. Despite potential glycogenolysis-dependent increase in glucose concentration in UW during storage (FIG. 30B), a significant depletion of renal HEP stores was apparent, in contrast to obvious preservation of energy state in Somah kidneys (FIG. 31). This suggests that UW kidneys were highly catabolic, leading to loss of HEP's and tissue injury, (FIG. 29). In contrast, greater oxidative phosphorylation of glucose in Somah-stored kidneys, facilitates greater HEP generation (for equivalent glucose molecules), than by anaerobic glycolysis alone thus enhancing the organs energy state during storage. Without being bound to theory, the low HEP levels observed in DCD UW-kidneys, could predictably result in delayed graft function (DGF) at the least, and even primary non-function (PNF). Therefore, despite the predominant use as preservation solution for explanted kidneys, UW may not provide optimal conditions for extended storage of DCD (or BHD) kidneys. In contrast, preservation in Somah may provide a viable alternative.

The blood vessels (capillaries) form the bulk of renal cortical tissue while tubular structures predominate in renal medulla. While glomerular tuft collapse within 6 hour storage in Ilistidine-Tryptophan-Ketoglutarate (IITK) solution has been reported (Kallerhoff M, Blech M, Kehrer G, Kleinert H, Langheinrich M, et al. (1987) Effects of glucose in protected ischemic kidneys. Urol Res 15: 215-222), such drastic glomerular change was not observed in either Somah or UW stored kidneys, at any timepoint (FIG. 29). However, a steady decline in expression of eNOS (important in vasomotor function), von-Willebrands factor (vWF; marker for blood vessel endothelium) and erythropoietin (EPO; marker of specialized peritubular epithelial cells), during 72 hour DCD kidney storage in UW, suggests subtle damage to both vascular and tubular structures (FIG. 32). This is consistent with histological findings of increase in tubular nuclear hyperchromacity observed in UWstored kidneys. In contrast, the expression of all investigated proteins including caveolin, eNOS, vWF and EPO were unaltered during the same periods of observation in Somrah-preserved kidneys suggesting both cortical and tubular renal tissue preservation.

This Example provides evidence that use of Somah for static preservation of DCD kidneys may potentially decrease the incidence of DGF, PNF and improve graft life upon transplantation.

1. A composition for preserving or resuscitating biological tissue or organs comprising: a physiological salt solution, glutathione, ascorbic acid, and adenosine, wherein the physiological salt solution comprises at least 20 mM potassium ions and at least 37 mM magnesium ions, wherein said composition preserves or resuscitates said biological tissue or organs at a temperature of 10-21±4° C.

2. A composition for preserving mammalian organs comprising: a physiological salt solution, glutathione, ascorbic acid, and adenosine, wherein the composition is maintained at a temperature of 10-21±4° C.

3. The composition of claim 1, wherein the composition is maintained at a temperature of 10-21±4° C.

4. The composition of claim 2, wherein the physiological salt solution comprises at least 20 mM potassium ions and at least 37 mM magnesium ions.

5. The composition of claim 1, further comprising insulin.

6. The composition of claim 5, wherein insulin is added to the composition just prior to use.

7. The composition of claim 1, wherein the physiological salt solution comprises one or more salts selected from the group consisting of potassium phosphate, potassium chloride, sodium chloride, sodium bicarbonate, calcium chloride, sodium phosphate, magnesium chloride, magnesium sulfate.

8. The composition of claim 1, comprising 0.4-10 mM of potassium phosphate.

9. The composition of claim 1, comprising 4-65 mM of potassium chloride.

10. The composition of claim 1, comprising 80-135 mM sodium chloride.

11. The composition of claim 1, comprising 2-25 mM sodium bicarbonate.

12. The composition of claim 1, comprising 0-1.5 mM calcium chloride.

13. The composition of claim 1, comprising 0.15-30 mM sodium phosphate.

14. The composition of claim 1, comprising 0.5-45 mM magnesium chloride.

15. The composition of claim 1, comprising 0.5-1.5 mM magnesium sulfate.

16. The composition of claim 1, further comprising about 2.5-5 mM creatine.

17. The composition of claim 1, further comprising about 0.001-0.5 mM dichloroacetate.

18. The composition of claim 1, further comprising about 0.5-2 mM orotic acid.

19. The composition of claim 1, further comprising about 11-25 mM of a sugar.

20. The composition of claim 19, wherein the sugar is glucose or dextrose.

21. The composition of claim 1, further comprising about 2-10 mM arginine.

22. The composition of claim 1, further comprising about 0.001-10 mM malic acid.

23. The composition of claim 1, further comprising about 1-10 mM citrulline.

24. The composition of claim 1, further comprising about 0.001-10 mM citrulline malate.

25. The composition of claim 1, further comprising about 5-10 mM carnosine.

26. The composition of claim 1, further comprising about 5-10 mM carnitine.

27.-34. (canceled)

35. A method for producing a composition for storing, preserving or resuscitating biological tissue or organs comprising combining a physiological salt solution, glutathione, ascorbic acid, and adenosine, wherein the physiological salt solution comprises at last 20 mM potassium ions and at least 37 mM magnesium ions.

36.-108. (canceled)

109. A method for inducing cardioplegia during open heart surgery or during heart donor removal surgery, the method comprising contacting the heart with a solution comprising a physiological salt solution, glutathione, ascorbic acid, and adenosine, wherein the physiological salt solution comprises at least 20 mM potassium ions or the physiological salt solution comprises 4-65 mM potassium ions and 1.5-45 mM magnesium ions.

110. A method for preserving donor lungs prior to transplantation surgery, the method comprising contacting the lungs with a solution comprising a physiological salt solution, glutathione, ascorbic acid, and adenosine, wherein the physiological salt solution comprises 4-65 mM potassium ions and 1.5-45 mM magnesium ions.

111.-116. (canceled)
